(19) United States
(12) Patent Application Publication
(10) Pub. No.: US 2013/0072437 A1
(43) Pub. Date: Mar. 21, 2013

(54) NEUREGULIN ISOFORMS,NEUREGULIN POLYPEPTIDES AND USES THEREOF

(71) Applicants: Thierry Baussant, Bellearde sur Valserine (FR); Daniel Bach, Geneve (CH); André Schrattenholz, Mainz (DE)

(72) Inventors: Thierry Baussant, Bellearde sur Valserine (FR); Daniel Bach, Geneve (CH); André Schrattenholz, Mainz (DE)

(73) Assignee: MIND-NRG SA, Genève (CH)

(21) Appl. No.: 13/700,209

(22) PCT Filed: May 27, 2011
(86) PCT No.: PCT/EP2011/058769

§ 371 Date: Nov. 27, 2012

(30) Foreign Application Priority Data
May 28, 2010 (US) 61349451

(51) Int. Cl. A61K 38/17; C12N 5/07; G01N 33/68; C07K 19/00

(52) U.S. Cl. CPC A61K 38/17; C07K 19/00; C12N 5/06; G01N 33/6896
USPC 514/17.5; 530/350; 514/1.1; 435/377; 435/455; 530/387.9; 435/7.4; 435/7.1; 436/501; 536/23.5; 514/17.8; 514/17.7; 514/17.6; 514/18.1

(57) ABSTRACT

The present invention relates to new therapeutic and diagnostic uses of soluble neuregulin-1 isoforms and polypeptides, particularly neurological disorders.

The present invention relates to new therapeutic and diagnostic uses of soluble neuregulin-1 isoforms and polypeptides, particularly neurological disorders.

### BACKGROUND OF THE INVENTION

The neuregulin (Nrg) family of growth and differentiation factors plays a crucial role in development and plasticity of the nervous system. Four genes (NRG-1 to NRG-4) are translated to diverse transmembrane and soluble isoforms. NRG-1 encodes 15 known Nrg1 isoforms with distinct time- and tissue-specific expression patterns. The extracellular domain (ECD) of Nrg1 is cleaved by β-amyloid converting enzyme-1 and released into the intercellular space to act as paracrine trophic factor. The ECD contains an epidermal growth factor (EGF)-like motive to activate ErbB3 and ErbB4 receptors by dimerization and tyrosin phosphorylation. ErbB4 is a functional receptor tyrosine kinase, while ErbB3 depends on hetero-dimerization to transduce signals.

Nrg1 has been genetically linked to schizophrenia, a neurodevelopmental mental disorder with imbalances in dopaminergic neurotransmission. Several lines of evidence suggest that Nrg1 affects dopamine-signaling. In fact, human and rodent midbrain dopaminergic neurons highly express ErbB4 throughout development into adulthood. An N-terminally truncated ECD of human Nrg1β₁ (nucleotides 46-634, 25.4 kDa) passes the immature blood-brain barrier (BBB) in neonatal mice, activates midbrain ErbB4 receptors, increases the enzymatic activity of tyrosine hydroxylase (TH, the rate-limiting enzyme of dopamine biosynthesis) and induces a persistent hyper-dopaminergic state; in this work, Nrg1β₁-treatment coincided with the postnatal phase of ontogenic cell death and axonal differentiation of the mesencephalic dopaminergic system, suggesting that Nrg1β₁ acts as neurotrophic factor during development.

Also in adult rodents, direct intracerebral infusion of the entire ECD (Ser2-Lys246, 26.9 kDa) of human Nrg1β₁ into the hippocampus or of the EGF-like domain only (Thr176-Lys246, 8 kDa) into the striatum transiently increases local dopamine release, indicating that some reactivity of the dopaminergic system to Nrg1β₁persists into adulthood.

Since the adult dopaminergic system is subject to progressive degeneration in various neurological disorders, e.g., Parkinson's disease (PD), leading to a disabling hypokinetic-rigid syndrome'¹⁵, there is a need in the art to provide new therapeutic strategies promoting neuroprotection and preventing neurodegeneration resulting in a loss of neurons.

### SUMMARY OF THE INVENTION

Based on in vivo, in vitro and in silico data the inventors have found that neuregulin-1 isoforms and neuregulin polypeptides, e.g. of Nrg1β₁ as described herein, are (i) capable of providing neuroprotection of, e.g., dopaminergic neurons, (ii) exhibit an improved receptor binding affinity and/or (iii) are capable of inducing cell differentiation of, e.g., erbB4- and/or erbB3-expressing cells that do not express neuromelanin and tyrosine hydroxylase. These cells were shown to transform into e.g. dopaminergic neurons when contacted with a polypeptide of the invention.

Thus, the invention provides in a first aspect a polypeptide, wherein the polypeptide comprises or consists of an EGF-like domain (EGFLD1) selected from the group consisting of SEQ ID NO: 140-146 (i.e. SEQ ID NO: 140, 141, 142, 143, 144, 145 and 146), wherein said EGF-like domain may comprise up to five single amino acid deletions, insertions and/or mutations and wherein said EGF-like domain optionally comprises up to 30 additional amino acids at its C- and/or N-terminus.

Also provided as a second aspect is a pharmaceutical composition comprising a polypeptide of the invention.

A further aspect of the invention relates to a polypeptide of the invention for use in the prophylaxis or treatment of a neurological condition.

Also provided is the use of soluble neuregulin isoform as described herein, of a polypeptide according to the invention or of a polynucleotide encoding said polypeptide for inducing differentiation of a cell.

On the basis of the experimental evidence provided in the examples below, it is a further aspect of the invention to provide a method for producing dopaminergic neurons comprising the step of
a) contacting a non-neuronal cell with a neuregulin isoform of the invention and/or with a polypeptide of the invention.

A further aspect of the invention is an antibody capable of specifically binding to a protein selected from the group consisting of 14-3-3-zeta (SEQ ID NOs:58, 133), 14-3-3-epsilon (SEQ ID NOs:59, 134), N-ethylmaleimide sensitive factor (SEQ ID NOs:50, 124), Aldolase A, fructose-bisphosphate (SEQ ID NOs:2, 68); Aldolase C, fructose-bisphosphate (SEQ ID NO:3, 69); Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate-ammonia ligase (Glutamine synthetase) (SEQ ID NOs:10, 78, 79); Dihydrolipoamide S-acetyltransferase (E2 component of pyruvate dehydrogenase complex) (SEQ ID NOs:11, 80, 66); Isocitrate dehydrogenase 3 (NAD+) alpha, isoform CRA_e (SEQ ID NOs:12, 81); Malate dehydrogenase, cytoplasmic (SEQ ID NOs:13, 82); NADH dehydrogenase (ubiquinone) 1 alpha subcomplex, 8 (SEQ ID NOs:14, 83); NADH dehydrogenase (ubiquinone) Fe—S protein 1 (SEQ ID NOs:15, 84, 67); NADH dehydrogenase (ubiquinone) Fe—S protein 8 (SEQ ID NOs:16, 85); Ubiquinol-cytochrome-c reductase complex core protein 1 (SEQ ID NOs:17, 86); ATP synthase, H+ transporting, mitochondrial FO complex, subunit d (SEQ ID NOs:18, 87, 88); Creatine kinase, brain (SEQ ID NOs:19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+ transporting, lysosomal V1 subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal hydrolase L1, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106); Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110); Neurofilament, light polypeptide (SEQ ID NOs:38, 111); Glial fibrillary acidic protein (SEQ ID NOs:39, 112, 113); Tubulin, alpha 1B

(SEQ ID NOs:40, 114); Tubulin, beta (SEQ ID NOs:41, 115); Tubulin, beta 3 (SEQ ID NOs:42, 116); Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1B, member RAS oncogene family; isoform CRA_a (SEQ ID NOs:46, 120); RAB3A, member RAS oncogene family (SEQ ID NOs:47, 121); RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135); Peroxiredoxin 3 (SEQ ID NOs:61, 136); Pyridoxal (pyridoxine, vitamin B6) kinase (SEQ ID NOs:62, 137); and Guanine nucleotide binding protein, alpha o isoform B (SEQ ID NOs:63, 138, 139)
for the use in diagnosing a disease.

As a further aspect the invention provides a method of diagnosing a disease comprising (i) determining in vitro in an isolated tissue explant or an isolated body fluid of a subject the quantity of a protein having at least 90% amino acid sequence identity over its entire length with a protein selected from the group consisting of 14-3-3-zeta (SEQ ID NOs:58, 133), 14-3-3-epsilon (SEQ ID NOs:59, 134), N-ethylmaleimide sensitive factor (SEQ ID NOs:50, 124), Aldolase A, fructose-bisphosphate (SEQ ID NOs:2, 68); Aldolase C, fructose-bisphosphate (SEQ ID NO:3, 69); Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate-ammonia ligase (Glutamine synthetase) (SEQ ID NOs:10, 78, 79); Dihydrolipoamide S-acetyltransferase (E2 component of pyruvate dehydrogenase complex) (SEQ ID NOs:11, 80, 66); Isocitrate dehydrogenase 3 (NAD+) alpha, isoform CRA_e (SEQ ID NOs:12, 81); Malate dehydrogenase, cytoplasmic (SEQ ID NOs:13, 82); NADH dehydrogenase (ubiquinone) 1 alpha subcomplex, 8 (SEQ ID NOs:14, 83); NADH dehydrogenase (ubiquinone) Fe—S protein 1 (SEQ ID NOs:15, 84, 67); NADH dehydrogenase (ubiquinone) Fe—S protein 8 (SEQ ID NOs:16, 85); Ubiquinol-cytochrome-c reductase complex core protein 1 (SEQ ID NOs:17, 86); ATP synthase, H+ transporting, mitochondrial F0 complex, subunit d (SEQ ID NOs:18, 87, 88); Creatine kinase, brain (SEQ ID NOs:19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+ transporting, lysosomal V1 subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal hydrolase L1, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106); Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110); Neurofilament, light polypeptide (SEQ ID NOs:38, 111); Glial fibrillary acidic protein (SEQ ID NOs:39, 112, 113); Tubulin, alpha 1B (SEQ ID NOs:40, 114); Tubulin, beta (SEQ ID NOs:41, 115); Tubulin, beta 3 (SEQ ID NOs:42, 116); Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1B, member RAS oncogene family; isoform CRA_a (SEQ ID NOs:46, 120); RAB3A, member RAS oncogene family (SEQ ID NOs:47, 121); RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135); Peroxiredoxin 3 (SEQ ID NOs:61, 136); Pyridoxal (pyridoxine, vitamin B6) kinase (SEQ ID NOs:62, 137); and Guanine nucleotide binding protein, alpha o isoform B (SEQ ID NOs:63, 138, 139) or a polynucleotide encoding said protein; and

(ii) optionally determining whether the amount of protein differs from the amount of the corresponding protein quantified in a healthy subject; and

(iii) optionally correlating a changed expression of said protein with a neurological disease.

In a further aspect the invention also provides a polynucleotide encoding a polypeptide of the invention.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland) and as described in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001.

Throughout this specification and the claims which follow, unless the context requires otherwise, the word “comprise”, and variations such as “comprises” and “comprising”, will be understood to imply the inclusion of a stated feature, integer or step or group of features, integers or steps but not the exclusion of any other feature, integer or step or group of integers or steps. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In part, the present invention is based on the surprising finding that a soluble neuregulin-1 isoform, e.g. Nrg1β₁-ECD as described herein, is capable of providing neuroprotection of, e.g., dopaminergic neurons and/or of inducing differentiation of, e.g., erbB4- and/or erbB3-expressing cells that do not express neuromelanin and tyrosine hydroxylase and/or non-neuronal cells, such as glial cells, particularly astrocytes, oligondentrocytes, ependymal cells, radio glial cells, Schwann cells, satellite cells and/or enteric glia cells in, e.g., dopaminergic neurons. In the case of differentiation of such cells in dopaminergic neurons, the increase in such neurons will increase the endogenous dopamine production. Such an increase in endogenous dopamine and/or the neuroprotective effect of neuregulin-1 are particularly useful for the symptomatic relief of patients suffering from Parkinson's disease. Without being bound to any theory, the inventors of the present invention believe that the new curative effect of neuregulin-1, i.e., neuroprotection and/or neuronal differentiation, is effected by the induction of tyrosine hydroxylase in non-neuronal cells, preferably erbB4- or erbB3-expressing cells. It is further believed that the duality of neuroprotection and induction of neuronal differentiation is the key for a new therapy, e.g., healing, for Parkinson's disease.

Furthermore, the present invention is based on the unexpected finding as deduced from in silico experiments that (i) small fragments of the extracellular domain of neuregulin are sufficient to bind erbB4- or erbB3-receptors and that (ii) polypeptides comprising multiple copies of selected domains of the neuregulin protein as described herein below, e.g. of neuregulin-1 protein show not only an increased binding affinity to the erbB4- or erbB3-receptors but also an enrichment near cells which naturally express erbB4- or erbB3-receptors. These improved polypeptides of the invention can thus be administered in smaller amounts to a subject such as a human patient and still be pharmaceutically effective, i.e. still have the same therapeutic effect as a polypeptide of the prior art that is administered at a larger dose. Smaller dosage forms are not only cheaper to produce but also provide the advantage that possible side-effects can be minimized as the therapeutic polypeptides specifically accumulate at the target cells and bind there with improved affinity to the mentioned receptors.

The present invention also provides a soluble neuregulin-1 isoform or a nucleic acid molecule encoding a soluble neuregulin-1 isoform all as described herein for the prevention, amelioration and/or treatment of a neurological disorder by induction of neuronal differentiation and/or neuroprotection. In a preferred embodiment, a neurological disorder is selected from the group consisting of schizophrenia, in particular cognition-related aspects of schizophrenia; Parkinson's disease; Alzheimer's disease; Multiple Sclerosis (MS); Amyotrophic Lateral Sclerosis (ALS); epilepsy; stroke; traumatic brain injury; spinal chord injury; bipolar disorders; depression; frontotemporal dementia; seizures; ischemia; neuropathy, particularly diabetic neuropathy; neuralgia; neuropathic pain; and inclusion-body myopathy. In a particularly preferred embodiment the neurological disorder is Parkinson's disease or bipolar disorder.

The present invention further provides a soluble neuregulin-1 isoform or a nucleic acid molecule encoding a soluble neuregulin-1 isoform all as described herein for the prevention, amelioration and/or treatment of a disorder associated with a loss of neurons, such as a neurological disorder, e.g., a neurological disorder selected from the group consisting of schizophrenia, in particular cognition-related aspects of schizophrenia; Parkinson's disease; Alzheimer's disease; Multiple Sclerosis (MS); Amyotrophic Lateral Sclerosis (ALS); epilepsy; stroke; traumatic brain injury; spinal chord injury; bipolar disorders; depression; frontotemporal dementia; seizures; ischemia; neuropathy, particularly diabetic neuropathy; neuralgia; neuropathic pain; and inclusion-body myopathy. In a particularly preferred embodiment the loss of neurons is associated with the neurological disorder Parkinson's disease. In a further preferred embodiment the loss of neurons is prevented by induction of neuronal differentiation and/or neuroprotection as described herein. In a preferred embodiment of the invention, the loss of neurons is the result of excitotoxicity, preferably glutamate-induced excitotoxicity as described in Schrattenholz et al, 2006, Current Topics in Medical Chemistry 6, 663-586.

In a further preferred embodiment of the invention, the neuronal differentiation as described herein is induced in erbB4- and/or erbB3-expressing cells that do not express neuromelanin and tyrosine hydroxylase and/or non-neuronal cells, such as glial cells, particularly astrocytes, oligondentrocytes, ependymal cells, radio glial cells, Schwann cells, satellite cells and/or enteric glia cells.

In a further embodiment of the invention, the neuronal differentiation is induced by altering the expression level of one or more proteins of Table 2 as described herein, e.g., of Aldolase A, fructose-bisphosphate (SEQ ID NOs:2, 68); Aldolase C, fructose-bisphosphate (SEQ ID NO:3, 69); Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate-ammonia ligase (Glutamine synthetase) (SEQ ID NOs:10, 78, 79); Dihydrolipoamide S-acetyltransferase (E2 component of pyruvate dehydrogenase complex) (SEQ ID NOs:11, 80, 66); Isocitrate dehydrogenase 3 (NAD+) alpha, isoform CRA_e (SEQ ID NOs:12, 81); Malate dehydrogenase, cytoplasmic (SEQ ID NOs:13, 82); NADH dehydrogenase (ubiquinone) 1 alpha subcomplex, 8 (SEQ ID NOs:14, 83); NADH dehydrogenase (ubiquinone) Fe—S protein 1 (SEQ ID NOs:15, 84, 67); NADH dehydrogenase (ubiquinone) Fe—S protein 8 (SEQ ID NOs:16, 85); Ubiquinol-cytochrome-c reductase complex core protein 1 (SEQ ID NOs:17, 86); ATP synthase, H+ transporting, mitochondrial FO complex, subunit d (SEQ ID NOs:18, 87, 88); Creatine kinase, brain (SEQ ID NOs:19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+ transporting, lysosomal V1 subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal hydrolase L1, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106); Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110); Neurofilament, light polypeptide (SEQ ID NOs:38, 111); Glial fibrillary acidic protein (SEQ ID NOs:39, 112, 113); Tubulin, alpha 1B (SEQ ID NOs:40, 114); Tubulin, beta (SEQ ID NOs:41, 115); Tubulin, beta 3 (SEQ ID NOs:42, 116); Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1B, member RAS oncogene family; isoform CRA_a (SEQ ID NOs:46, 120); RAB3A, member RAS oncogene family (SEQ ID NOs:47, 121); RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135); Peroxiredoxin 3 (SEQ ID NOs:61, 136); Pyridoxal (pyridoxine, vitamin B6) kinase (SEQ ID NOs:62, 137); Guanine nucleotide binding protein, alpha o isoform B (SEQ ID NOs:63, 138, 139); 14-3-3-zeta (SEQ ID NOs:58, 133); 14-3-3-epsilon (SEQ ID NOs:59, 134); and N-ethylmaleimide sensitive factor (SEQ ID NOs:50, 124).

In one embodiment, the alteration of the expression level is a decrease in expression of a protein of Table 2 as described herein and as selected from the group consisting of 14-3-3-zeta (SEQ ID NOs:58, 133), 14-3-3-epsilon (SEQ ID NOs:59, 134), and N-ethylmaleimide sensitive factor (SEQ ID NOs:50, 124).

In another embodiment, the alteration of the expression level is an increase in expression of a protein of Table 2 as described herein and as selected from the group consisting of Aldolase A, fructose-bisphosphate (SEQ ID NOs:2, 68); Aldolase C, fructose-bisphosphate (SEQ ID NO:3, 69); Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate-ammonia ligase (Glutamine synthetase) (SEQ ID NOs:10, 78, 79); Dihydrolipoamide S-acetyltransferase (E2 component of pyruvate dehydrogenase complex) (SEQ ID NOs:11, 80, 66); Isocitrate dehydrogenase 3 (NAD+) alpha, isoform CRA_e (SEQ ID NOs:12, 81); Malate dehydrogenase, cytoplasmic (SEQ ID NOs:13, 82); NADH dehydrogenase (ubiquinone) 1 alpha subcomplex, 8 (SEQ ID NOs:14, 83); NADH dehydrogenase (ubiquinone) Fe—S protein 1 (SEQ ID NOs:15, 84, 67); NADH dehydrogenase (ubiquinone) Fe—S protein 8 (SEQ ID NOs:16, 85); Ubiquinol-cytochrome-c reductase complex core protein 1 (SEQ ID NOs:17, 86); ATP synthase, H+ transporting, mitochondrial FO complex, subunit d (SEQ ID NOs:18, 87, 88); Creatine kinase, brain (SEQ ID NOs:19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+ transporting, lysosomal V1 subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal hydrolase L1, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106); Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110); Neurofilament, light polypeptide (SEQ ID NOs:38, 111); Glial fibrillary acidic protein (SEQ ID NOs:39, 112, 113); Tubulin, alpha 1B (SEQ ID NOs:40, 114); Tubulin, beta (SEQ ID NOs:41, 115); Tubulin, beta 3 (SEQ ID NOs:42, 116); Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1B, member RAS oncogene family; isoform CRA_a (SEQ ID NOs:46, 120); RAB3A, member RAS oncogene family (SEQ ID NOs:47, 121); RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135); Peroxiredoxin 3 (SEQ ID NOs:61, 136); Pyridoxal (pyridoxine, vitamin B6) kinase (SEQ ID NOs:62, 137); and Guanine nucleotide binding protein, alpha o isoform B (SEQ ID NOs:63, 138, 139). Particularly preferred is the alteration of the expression level that is an increase in expression of the protein Dihydropyrimidinase-like 2 (SEQ ID NO:43, 117). Particularly preferred is the alteration of the expression level that is an increase in expression of the protein Valosin containing protein, isoform_b (SEQ ID NO:28, 99).

The term “a protein of Table 2” as used throughout the application refers to a mammalian protein, most preferably a mouse, a rat or a human protein. The term “protein of Table 2” as used herein also includes variants of these proteins such as allelic variants, splice variants or variants, particularly human variants with 99%, 98%, 97%, 96%, 95%, 93%, 90%, 85% or 80% identity to the mouse proteins described herein, e.g. the mouse protein referred to in Table 2 as well as derivatives functionally active or fragments of these proteins. The term “% identity” as used in the above context and also as used generally throughout this specification refers to the %-identity that is identified on the basis of the BLAST program (Altschul, S. F., Gish, W., Miller, W., Myers, E. W. & Lipman, D. J. (1990) “Basic local alignment search tool.” J. Mol. Biol. 215:403-410; Gish, W. & States, D. J. (1993) “Identification of protein coding regions by database similarity search.” Nature Genet. 3:266-272; Madden, T. L., Tatusov, R. L. & Zhang, J. (1996) “Applications of network BLAST server” Meth. Enzymol. 266:131-141; Altschul, S. F., Madden T. L., Schaffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402) by using the database RefSeq protein. In one preferred embodiment the percent identity is determined with respect to the sequence which is longest, i.e. the longer of the two sequences which are compared to each other is preferably the reference sequence. The proteins of Table 2 may be used for the prevention, amelioration and/or treatment of a neurological disorder. Proteins of Table 2 that may be used in the context of the invention are selected from the group consisting of Aldolase A, fructose-bisphosphate (SEQ ID NOs:2, 68); Aldolase C, fructose-bisphosphate (SEQ ID NO:3, 69); Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate-ammonia ligase (Glutamine synthetase) (SEQ ID NOs:10, 78, 79); Dihydrolipoamide S-acetyltransferase (E2 component of pyruvate dehydrogenase complex) (SEQ ID NOs:11, 80, 66); Isocitrate dehydrogenase 3 (NAD+) alpha, isoform CRA_e (SEQ ID NOs:12, 81); Malate dehydrogenase, cytoplasmic (SEQ ID NOs:13, 82); NADH dehydrogenase (ubiquinone) 1 alpha subcomplex, 8 (SEQ ID NOs:14, 83); NADH dehydrogenase (ubiquinone) Fe—S protein 1 (SEQ ID NOs:15, 84, 67); NADH dehydrogenase (ubiquinone) Fe—S protein 8 (SEQ ID NOs:16, 85); Ubiquinol-cytochrome-c reductase complex core protein 1 (SEQ ID NOs:17, 86); ATP synthase, H+ transporting, mitochondrial F0 complex, subunit d (SEQ ID NOs:18, 87, 88); Creatine kinase, brain (SEQ ID NOs:19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+ transporting, lysosomal V1 subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal hydrolase L1, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106); Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110); Neurofilament, light polypeptide (SEQ ID NOs:38, 111); Glial fibrillary acidic protein (SEQ ID NOs:39, 112, 113); Tubulin, alpha 1B (SEQ ID NOs:40, 114); Tubulin, beta (SEQ ID NOs:41, 115); Tubulin, beta 3 (SEQ ID NOs:42, 116); Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1B, member RAS oncogene family; isoform CRA_a (SEQ ID NOs:46, 120); RAB3A, member RAS oncogene family (SEQ ID NOs:47, 121); RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135); Peroxiredoxin 3 (SEQ ID NOs:61, 136); Pyridoxal (pyridoxine, vitamin B6) kinase (SEQ ID NOs:62, 137); Guanine nucleotide binding protein, alpha o isoform B (SEQ ID NOs:63, 138, 139); 14-3-3-zeta (SEQ ID NOs:58, 133); 14-3-3-epsilon (SEQ ID NOs:59, 134); and N-ethylmaleimide sensitive factor (SEQ ID NOs:50, 124). Particularly preferred is the neuronal differentiation that is induced by increasing the expression level of Dihydropyrimidinase-like 2 (SEQ ID NO: 43, 117). Particularly preferred is also the neuronal differentiation that is induced by increasing the expression level of Valosin containing protein, isoform CRA_b (SEQ ID NOs: 28, 99). Alternatively, the neuronal differentiation is preferred that is induced by decreasing the expression level of 14-3-3-zeta (SEQ ID NOs: 58, 133), 14-3-3-epsilon (SEQ ID NOs: 59, 134) and/or N-ethylmaleimide sensitive factor (SEQ ID NOs: 50, 124).

The invention further relates to a soluble neuregulin-1 isoform which is preferably a human neuregulin-1 isoform, e.g., a recombinant isoform comprising the primary amino acid sequence of a naturally occurring human neuregulin-1 isoform or a sequence which has a identity of at least 90%, preferably at least 95% and most preferably of at least 98% based on the total length of the recombinant isoform.

The invention also includes variants of a soluble neuregulin-1 isoform such as allelic variants or splice variants as well as derivatives or fragments of these proteins. Preferably, said derivative is a glycosylated form of the protein.

The soluble neuregulin-1 isoform may be a neuregulin-1 Type I, Type II, Type III, Type IV, Type V or Type VI isoform, preferably a neuregulin-1β₁ isoform, a neuregulin-1 α isoform or a Sensory and motor neuron-derived factor (SMDF) isoform, particularly a neuregulin-1β₁ isoform and more particularly a human neuregulin-1β₁ isoform.

In a preferred embodiment, the soluble neuregulin-1 isoform is characterized in that it passes the blood brain barrier, e.g., a neuregulin-1β₁ isoform.

The soluble neuregulin-1 isoform comprises at least a portion of the extracellular domain of neuregulin-1 or fragments thereof, particularly the EGF-like domain or the EGF-like domain, the IgG-like domain and the heparan sulfate binding motif, particularly an isoform that comprises or is SEQ ID NO:1. In a preferred embodiment, the soluble neuregulin-1 isoform comprises:
(a) nucleotides 46-634 of SEQ ID NO:64,
(b) amino acids 176-246 of SEQ ID NO:1, and/or
(c) amino acids 2-246 of SEQ ID NO:1 (also described as NRG-β₁-ECD herein).

In a preferred embodiment of the polypeptide of the invention described herein below, the polypeptide comprises:
(a) a polypeptide encloded by nucleotides 46-634 of SEQ ID NO:64,
(b) a polypeptide consisting of amino acids 176-246 of SEQ ID NO:1, and/or
(c) a polypeptide consiting of amino acids 2-246 of SEQ ID NO:1,

wherein the polypeptide in (a), (b) and (c) may comprise up to 13 single amino acid deletions, insertions and/or mutations.

In a particularly preferred embodiment, the soluble neuregulin-1 isoform comprises amino acids 2-246 of SEQ ID NO:1.

The invention also provides a nucleic acid molecule encoding a soluble neuregulin-1 isoform as described herein, preferably a nucleic acid molecule comprising SEQ ID NO: 64, or encoding a protein of Table 2 as described herein as well as a vector comprising such a nucleic acid molecule, e.g., an expression vector. The nucleic acid molecule or the vector all as described herein may be transfected into a cell, which may be a prokaryotic cell, e.g., an E. coli cell, or an eukaryotic cell.

In one embodiment of the invention, the nucleic acid molecule encoding the soluble neuregulin-1 isoform or the nucleic acid molecule encoding a protein of Table 2 all as described herein is for the therapeutic uses as described herein, e.g., for the prevention, amelioration and/or treatment of a neurological disorder by induction of neuronal differentiation and/or neuroprotection as described herein or for the prevention, amelioration and/or treatment of a disorder associated with a loss of neurons as described herein, preferably for the prevention, amelioration and/or treatment of Parkinson's disease.

The invention further relates to the soluble neuregulin-1 isoform, a nucleic acid molecule encoding a soluble neuregulin-1 isoform, the protein of table 2, or a nucleic acid molecule encoding a protein of Table 2, all as described herein, in combination with a further active agent, e.g., an agent for the treatment of neurological conditions and/or neurological disorders such as Parkinson's disease, Alzheimer's disease, Multiple Sclerosis (MS), Amyotrophic Lateral Sclerosis (ALS), epilepsy, stroke, traumatic brain injury, spinal chord injury, psychotic disorders such as schizophrenia, bipolar disorders and depression, e.g. olanzapine or clozapine.

The invention further relates to a pharmaceutical composition comprising as active agent a soluble neuregulin-1 isoform, a nucleic acid molecule encoding a soluble neuregulin-1 isoform, a protein of Table 2 or a nucleic acid molecule encoding a protein of Table2, all as described herein and optionally a pharmaceutically active carrier.

The invention further provides a method for studying a neurological disorder, the molecular mechanism of, the physiological processes associated with a loss of neurons, or a disorder associated with a loss of neurons comprising:
(a) administering a neuregulin-1 isoform preferably as described herein, to a cell or a non-human vertebrate animal;
(b) subjecting said cell or an organ or tissue sample of said animal to a proteome analysis or gene expression analysis; and
(c) comparing the proteome analysis or the gene expression analysis to the respective analysis of a control cell or a control non-human animal.

In the above method, the neurological disorder may be selected from the group consisting of schizophrenia, in particular cognition-related aspects of schizophrenia; Parkinson's disease; Alzheimer's disease; Multiple Sclerosis (MS); Amyotrophic Lateral Sclerosis (ALS); epilepsy; stroke; traumatic brain injury; spinal chord injury; bipolar disorders; depression; frontotemporal dementia; seizures; ischemia; neuropathy, particularly diabetic neuropathy; neuralgia; neuropathic pain; and inclusion-body myopathy. In preferred embodiment, the neurological disorder is Parkinson's disease.

In the methods described herein the non-human vertebrate animal may be selected from the group consisting of mouse, rat, rabbit, hamster, bird, cat, sheep, bovine, and horse, preferably a mouse, and most preferably a mouse model for a neurological disorder, such as for Parkinson's disease, preferably by inducing neuronal death with 6-hydroxydopamine (6-OHDH) in wild-type mice or the transgenic mouse model A53T alpha-synuclein (Harvey B K, Wang Y, Hoffer B J. Transgenic rodent models of Parkinson's disease. Acta Neurochir Suppl. 2008;101:89-92; Chesselet M F. In vivo alpha-synuclein overexpression in rodents: a useful model of Parkinson's disease? Exp Neurol. 2008 January;209(1):22-7) or for Alzheimer's disease, preferably the APP/PS mouse model (Meyer-Luehmann, M.; Coomaraswamy, J.; Bolmont, T.; Kaeser, S.; Schaefer, C.; Kilger, E.; Neuenschwander, A.; Abramowski, D.; Frey, P.; Jaton, A. L.; Vigouret, J. M.; Paganetti, P.; Walsh, D. M.; Mathews, P. M.; Ghiso, J.; Staufenbiel, M.; Walker, L. C.; Jucker, M. (2006) Exogenous induction of cerebral beta-amyloidogenesis is governed by agent and host, Science 313, 1781-1784; Radde, R.; Bolmont, T.; Kaeser, S. A.; Coomaraswamy, J.; Lindau, D.; Stoltze, L.; Calhoun, M. E.; Jaggi, F.; Wolburg, H.; Gengler, S.; Haass, C.; Ghetti, B.; Czech, C.; Holscher, C.; Mathews, P. M.; Jucker, M. Abeta42-driven cerebral amyloidosis in transgenic mice reveals early and robust pathology (2006) EMBO Report 7, 940-946). Alternatively, the non-human animal model may be a wild-type animal.

The invention further provides a method for identifying and/or testing an agent that alters the expression and/or function of any one of the proteins of Table 2 or the nucleic acids encoding a protein of Table 2 comprising:
(a) administering said agent to a cell or a non-human vertebrate animal;
(b) measuring or monitoring the expression and/or function of said proteins or nucleic acid molecules encoding said proteins; and
(c) comparing the expression and/or function of said proteins or nucleic acid molecules to the expression and/or function of said proteins or nucleic acid molecules in a control cell or a control non-human vertebrate animal.

A particularly preferred protein of Table 2 in the above method is Dihydropyrimidinase-like 2 (SEQ ID NOs: 43, 117) and/or Valosin containing protein, isoform CRA_b (SEQ ID NOs: 28, 99). Another preferred protein of Table 2 in the above method is 14-3-3-zeta (SEQ ID NOs: 58, 133), 14-3-3-epsilon (SEQ ID NOs: 59, 134) and/or N-ethylmaleimide sensitive factor (SEQ ID NOs: 50, 124).

The expression of said proteins is measured by differential expression analysis with, e.g., isotope markers such as radioactive or stable isotopes which lead to a differential display. Alternatively, the expression of said proteins is measured with 2D gel-electrophoresis or mass spectrometry.

The expression of said nucleic acid molecules may be measured with DNA/RNA arrays, e.g. affymetrix.

Cells that may be used in the context of the methods described herein are LUHMES cells (Schildknecht, S.; Poltl, D.; Nagel, D. M.; Matt, F.; Scholz, D.; Lotharius, J.; Schmieg, N.; Salvo-Vargas, A.; Leist, M., 2009, Requirement of a dopaminergic neuronal phenotype for toxicity of low concentrations of 1-methyl-4-phenylpyridinium to human cells, Toxicol.Applied Pharmacol 241, 23-35) or any other neuronal cell, like a neuroblstoma cell, a primary culture of a neuronal cell and in particular include SHSY5Y cells (ATCC CRL-2266).

In the above described methods, the term “control cell” and “control non-human animal” refers to a cell or an animal that is used in a parallel experiment with identical conditions except for receiving said neuregulin-1 isoform or said agent.

Also the following items are within the ambit of the present invention:
A first item concerns a soluble neuregulin-1 isoform as described herein for the prevention, amelioration and/or treatment of a neurological disorder by induction of neuronal differentiation and/or neuroprotection.
The invention provides in a second item a soluble neuregulin-1 isoform as described herein for the prevention, amelioration and/or treatment of a disorder associated with a loss of neurons.
Item 3 provides the soluble neuregulin-1 isoform of item 2, wherein the loss of neurons is the result of excitotoxicity, preferably glutamate induced excitotoxicity.
In one embodiment, the invention provides as item 4 the soluble neuregulin-1 isoform of items 2 or 3, wherein the loss of neurons is prevented by induction of neuronal differentiation and/or neuroprotection.
In a further embodiment, the invention provides as item 5 the soluble neuregulin-1 isoform of item 1 or item 4, wherein the neuronal differentiation is induced in non-neuronal cells, such as glial cells, particularly astrocytes, oligondentrocytes, ependymal cells, radio glial cells, Schwann cells, satellite cells and/or enteric glia cells.
In a further embodiment, the invention provides as item 6 the soluble neuregulin-1 isoform of any one of items 1, 4 or 5, wherein the neuronal differentiation is induced in erbB4- and/or erbB3-expressing cells that do not express neuromelanin and tyrosine hydroxylase.
In a further embodiment, the invention provides as item 7 the soluble neuregulin-1 isoform of any one of items 1 and 4-6, wherein the neuronal differentiation is induced by altering the expression level of one or more proteins disclosed in Table 2.
In a further embodiment, the invention provides as item 8 the soluble neuregulin-1 isoform of item 7, wherein the alteration of the expression level is a decrease in expression of a protein selected from the group consisting of 14-3-3-zeta (SEQ ID NOs:58, 133), 14-3-3-epsilon (SEQ ID NOs:59, 134), and N-ethylmaleimide sensitive factor (SEQ ID NOs:50, 124).
In a further embodiment, the invention provides as item 9 the soluble neuregulin-1 isoform of item 7, wherein the alteration of the expression level is an increase in expression of a protein selected from the group consisting of Aldolase A, fructose-bisphosphate (SEQ ID NOs:2, 68); Aldolase C, fructose-bisphosphate (SEQ ID NO:3, 69); Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate-ammonia ligase (Glutamine synthetase) (SEQ ID NOs:10, 78, 79); Dihydrolipoamide S-acetyltransferase (E2 component of pyruvate dehydrogenase complex) (SEQ ID NOs:11, 80, 66); Isocitrate dehydrogenase 3 (NAD+) alpha, isoform CRA_e (SEQ ID NOs:12, 81); Malate dehydrogenase, cytoplasmic (SEQ ID NOs:13, 82); NADH dehydrogenase (ubiquinone) 1 alpha subcomplex, 8 (SEQ ID NOs:14, 83); NADH dehydrogenase (ubiquinone) Fe—S protein 1 (SEQ ID NOs:15, 84, 67); NADH dehydrogenase (ubiquinone) Fe—S protein 8 (SEQ ID NOs:16, 85); Ubiquinol-cytochrome-c reductase complex core protein 1 (SEQ ID NOs:17, 86); ATP synthase, H+ transporting, mitochondrial F0 complex, subunit d (SEQ ID NOs:18, 87, 88); Creatine kinase, brain (SEQ ID NOs:19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+ transporting, lysosomal V1 subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal hydrolase L1, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106); Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110); Neurofilament, light polypeptide (SEQ ID NOs:38, 111); Glial fibrillary acidic protein (SEQ ID NOs:39, 112, 113); Tubulin, alpha 1B (SEQ ID NOs:40, 114); Tubulin, beta (SEQ ID NOs:41, 115); Tubulin, beta 3 (SEQ ID NOs:42, 116); Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1B, member RAS oncogene family; isoform CRA_a (SEQ ID NOs:46, 120); RAB3A, member RAS oncogene family (SEQ ID NOs:47, 121); RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135); Peroxiredoxin 3 (SEQ ID NOs:61, 136); Pyridoxal (pyridoxine, vitamin B6) kinase (SEQ ID NOs:62, 137); and Guanine nucleotide binding protein, alpha o isoform B (SEQ ID NOs:63, 138, 139).
In a further embodiment, the invention provides as item 10 the soluble neuregulin-1 isoform of item 9, wherein said protein is dihydropyrimidinase-like 2 (SEQ ID NOs:43, 117) and/or valosin-containing protein, isoform CRA_b (SEQ ID NOs:28, 99).
A further aspect of the invention is item 11 which is a protein of Table 2 for the prevention, amelioration and/or treatment of a neurological disorder.
Also provided is item 12 which relates to a soluble neuregulin-1 isoform of any one of items 2-10, wherein the loss of neurons is associated with a neurological disorder.
Also provided is item 13 which is the soluble neuregulin-1 isoform of any one of items 1-10 or 12 or the protein of item 11, wherein the neurological disorder is selected from the group consisting of schizophrenia, in particular cognition-related aspects of schizophrenia; Parkinson's disease; Alzheimer's disease; Multiple Sclerosis (MS); Amyotrophic Lateral Sclerosis (ALS); epilepsy; stroke; traumatic brain injury; spinal chord injury; bipolar disorders; depression; frontotemporal dementia; seizures; ischemia; neuropathy, particularly diabetic neuropathy; neuralgia; neuropathic pain; and inclusion-body myopathy.
Item 14 is directed at a preferred embodiment of item 13, wherein the neurological disorder is Parkinson's disease.
Also provided is as item 15 the soluble neuregulin-1 isoform of any one of items 1-10 or 12-14, which is characterized in that it passes the blood brain barrier.
In one embodiment of the soluble neuregulin-1 isoform of any one of items 1-10 or 12-15 the neuregulin-1 isoform is a neuregulin-1β₁ isoform. This embodiment is also referred to as item 16.
In a further embodiment, item 17 relates to the soluble neuregulin-1 isoform of any one of items 1-10 or 12-16, which is characterized in that it comprises the extracellular domain of neuregulin-1 or fragments thereof, particularly the EGF-like domain or the EGF-like domain, the IgG-like domain and the heparan sulfate binding motif.
In a further embodiment, item 18 provides the soluble neuregulin-1 isoform of any one of items 1-10 or 12-17, wherein the isoform comprises SEQ ID NO:1.
In a further embodiment, item 19 provides the soluble neuregulin-1 isoform of any one of items 15-17, wherein the isoform comprises:
   (a) nucleotides 46-634 of SEQ ID NO:64,
   (b) amino acids 176-246 of SEQ ID NO:1, and/or
   (c) amino acids 2-246 of SEQ ID NO:1.
In a further embodiment, the invention provides as item 20 the soluble neuregulin-1 isoform of item 19 which comprises amino acids 2-246 of SEQ ID NO:1.
In a further embodiment, the invention provides as item 21 the soluble neuregulin-1 isoform of any one of items 1-10 or 12-20 or the protein of item 11 in combination with a further active agent.
In a further embodiment, the invention provides as item 22 the soluble neuregulin-1 isoform of item 21 or the protein of item 21, wherein the further active agent is an agent for the treatment of neurological conditions and/or neurological disorders.
In a further embodiment, item 23 provides the soluble neuregulin-1 isoform of item 22 or the protein of item 22, wherein the further agent is selected from compounds affecting catecholamine metabolism, acetylcholine esterase inhibitors, MAO-B- or COMT-inhibitors, Memantine-type channel blockers, dopamine or serotonine receptor agonists or antogonists, catecholamine or serotonine reuptake inhibitors or any type of antipsychotic medication like clozapine or olanzapine or gabapentin-like drugs in the treatments of Alzheimer's and Parkinson's diseases, schizophrenia, bipolar disorder, depression or other neurological conditions.
In a further embodiment, the invention provides as item 24 the soluble neuregulin-1 isoform of items 21 or 22 or protein of items 21 or 22, wherein the further agent is an agent for the treatment of psychotic disorders such as schizophrenia, bipolar disorders and depression, e.g. olanzapine or clozapine.
In a further embodiment, the invention provides as item 25 the soluble neuregulin-1 isoform of items 21 or 22 or the protein of items 21 or 22, wherein the further agent is an agent for the treatment of Parkinson's disease.
In a further embodiment, the invention provides as item 26 the soluble neuregulin-1 isoform of items 21 or 22 or the protein of items 21 or 22, wherein the further agent is an agent for the treatment of Alzheimer's disease.
In a further embodiment, the invention provides as item 27 the soluble neuregulin-1 isoform of items 21 or 22 or the protein of items 21 or 22, wherein the further agent is an agent for the treatment of Multiple Sclerosis (MS).
In a further embodiment, the invention provides as item 28 the soluble neuregulin-1 isoform of items 21 or 22 or the protein of items 21 or 22, wherein the further agent is an agent for the treatment of Amyotrophic Lateral Sclerosis (ALS).
In a further embodiment, the invention provides as item 29 the soluble neuregulin-1 isoform of items 21 or 22 or the protein of items 21 or 22, wherein the further agent is an agent for the treatment of epilepsy.
In a further embodiment, the invention provides as item 30 the soluble neuregulin-1 isoform of items 21 or 22 or the protein of items 21 or 22, wherein the further agent is an agent for the treatment of stroke.
In a further embodiment, the invention provides as item 31 the soluble neuregulin-1 isoform of items 21 or 22 or the protein of items 21 or 22, wherein the further agent is an agent for the treatment of traumatic brain injury.
In a further embodiment, item 32 provides the soluble neuregulin-1 isoform of items 21 or 22 or the protein of items 21 or 22, wherein the further agent is an agent for the treatment of spinal chord injury.
In a further aspect item 33 relates to a nucleic acid molecule encoding the soluble neuregulin-1 isoform of any one of items 15-20, preferably the nucleic acid molecule comprising SEQ ID NO:64.
In another aspect item 34 provides the nucleic acid molecule of item 33 for the uses of any one of items 1-10, 12-14 or 22-32.
Also provided is item 35 which is a nucleic acid molecule encoding a protein of Table 2 for the use of any one of items 11, 13, 14, or 22-32.
A further aspect, item 36 relates to a pharmaceutical composition comprising as active agent a soluble neuregulin-1 isoform of any one of items 15-20 and/or a nucleic acid molecule encoding a soluble neuregulin-1 isoform of item 33 and optionally a pharmaceutically active carrier.
In another aspect the invention provided item 37 which is a method for studying a neurological disorder, the molecular mechanism of, the physiological processes associated with a loss of neurons, or a disorder associated with a loss of neurons comprising:
   (a) administering a neuregulin-1 isoform to a cell or a non-human vertebrate animal;
   (b) subjecting said cell or an organ or tissue sample of said animal to a proteome analysis or gene expression analysis; and
   (c) comparing the proteome analysis or the gene expression analysis to the respective analysis of a control cell or a control non-human animal.
In one embodiment, the invention provides as item 38 the method of item 37, wherein the neurological disorder is selected from the group consisting of schizophrenia, in particular cognition-related aspects of schizophrenia; Parkinson's disease; Alzheimer's disease; Multiple Sclerosis (MS); Amyotrophic Lateral Sclerosis (ALS); epilepsy; stroke; traumatic brain injury; spinal chord injury; bipolar disorders; depression; frontotemporal dementia; seizures; ischemia; neuropathy, particularly diabetic neuropathy; neuralgia; neuropathic pain; and inclusion-body myopathy.
Item 39 relates to the method of items 37 or 38, wherein the neurological disorder is Parkinson's disease.
A preferred embodiment is item 40 which is the method of any one of items 37-39, wherein the non-human vertebrate animal is selected from the group consisting of mouse, rat, rabbit, hamster, bird, cat, sheep, bovine, and horse.
In a further embodiment, the invention provides as item 41 the method of item 40, wherein the non-human vertebrate animal is a mouse, preferably a mouse model for a neurological disorder.
In a further embodiment, item 42 provides the method of item 41, wherein said mouse model is for Parkinson's disease, preferably by inducing neuronal death with 6-hydroxydopamine (6-OHDA) or the A53T alpha synuclein transgenic mouse or for Alzheimer's disease, preferably the APP/PS mouse model.
Also within the ambit of the invention is item 43 which is, as also described herein above, a method for identifying and/or testing an agent that alters the expression and/or function of any one of the proteins of Table 2 or the nucleic acids encoding a protein of Table 2 comprising:
   (a) administering said agent to a cell or a non-human vertebrate animal;
   (b) measuring or monitoring the expression and/or function of said proteins or nucleic acid molecules encoding said proteins; and
   (c) comparing the expression and/or function of said proteins or nucleic acid molecules to the expression and/or function of said proteins or nucleic acid molecules in a control cell or a control non-human vertebrate animal.

Also the following aspects and preferred embodiments are within the ambit of the invention:

Before outlining these further aspects and embodiments, some additional definitions of terms frequently used in this specification are provided. These terms will, in each instance of its use, have the respectively defined meaning and preferred meanings

As used herein, the term “isolated” refers to a molecule which is substantially free of other molecules with which it is naturally associated with. An isolated molecule is thus free of other molecules that it would encounter or contact in a living animal in nature, i.e. outside an experimental setting. Preferably, the antibody or fragment thereof of the present invention is an isolated antibody or fragment thereof.

As used herein, the term “polypeptide” refers to both naturally occurring polypeptides and synthesized polypeptides that may include naturally or non-naturally occurring amino acids. Polypeptide can also be modified, e.g. can comprise a chemical modification of a side chain or a free amino or carboxy-terminus of a natural or non-naturally occurring amino acid. This chemical modification includes detectable labels, such as a fluorophore. A polypeptide may also comprise further modifications such as the side chain or a free amino or carboxy-terminus of an amino acid of the polypeptide may be modified by e.g. glycosylation, amidation, phosphorylation, ubiquitination, e.t.c. Such modification can be effected in vitro or in a host-cell i.e. in vivo, as is well known in the art of protein science. For example, a suitable chemical modification motif, e.g. glycosylation sequence motif present in the amino acid sequence of the polypeptide will cause it to be glycosylated in vivo. A polypeptide according to the invention has in a preferred embodiment not more than 300 amino acids, preferably not more than 244 amino acids and most preferably not more than 200 amino acids.

As used throughout this application, the phrase “single amino acid substitution, deletion and/or insertion” of a polypeptide generally refers to a modified version of the polypeptide, e.g. one amino acid of the polypeptide may be deleted, inserted and/or substituted. If the polypeptide comprises several single amino acid substitutions, deletions and/or insertions then the total number of such substitutions, deletions and/or insertions is indicated in each case. Said insertion is an insertion of the indicated number of single amino acids into the original polypeptide or protein. If the polypeptide comprises one or more single amino acid substitutions, said substitutions may in each case independently be a conservative or a non-conservative substitution, preferably a conservative substitution. In some embodiments, a substitution also includes the exchange of a naturally occurring amino acid with a not naturally occurring amino acid. In a most preferred embodiment, all substitutions are of conservative nature as further defined below. A conservative substitution comprises the substitution of one amino acid with another amino acid having a chemical property similar to the amino acid that is substituted. Preferably, the conservative substitution is a substitution selected from the group consisting of:
(i) a substitution of a basic amino acid with another, different basic amino acid;
(ii) a substitution of an acidic amino acid with another, different acidic amino acid;
(iii) a substitution of an aromatic amino acid with another, different aromatic amino acid;
(iv) a substitution of a non-polar, aliphatic amino acid with another, different non-polar, aliphatic amino acid; and
(v) a substitution of a polar, uncharged amino acid with another, different polar, uncharged amino acid.

A basic amino acid is preferably selected from the group consisting of arginine, histidine, and lysine. An acidic amino acid is preferably aspartate or glutamate. An aromatic amino acid is preferably selected from the group consisting of phenylalanine, tyrosine and tryptophane. A non-polar, aliphatic amino acid is preferably selected from the group consisting of glycine, alanine, valine, leucine, methionine and isoleucine. A polar, uncharged amino acid is preferably selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v).

If a polypeptide comprises one or an indicated number of single amino acid deletions, then said number of amino acids present in the reference polypeptide have been removed.

Reference will be made in the following to preferred amino acid sequences which are outlined in the table below:

| SEQ | | |
|---|---|---|
| ID | | |
| NO: | Amino Acid Sequence | Annotation |
| 140 | CPNEFTGDRCQNYVMASFYKHLGIEFME | Fragment of EGF-like domain of β1 |
| | | neuregulin 1 |
| 141 | CPNEFTGDRCQNYVMASFYK | Fragment of EGF-like domain of β2 |
| | | neuregulin 1 |
| 142 | CPNEFTGDRCQNYVMASFYSTSTPFLSLPE | Fragment of EGF-like domain of β3 |
| | | (long) neuregulin 1 |
| 143 | CPNEFTGDRCQNYVMASFYS | Fragment of EGF-like domain of β3 |
| | | (short) neuregulin 1 |
| 144 | CQPGFTGARCTENVPMKVQNQEK | Fragment of EGF-like domain of α |
| | | neuregulin 1 |
| 145 | CQPGFTGARCTENVPMKVQNQES | Fragment of EGF-like domain of α3 |
| | | neuregulin 1 |
| 146 | CQPGFTGARCTENVPMKVQNQEKHLGIEFIE | Fragment of EGF-like domain of |
| | | α1A neuregulin 1 |
| 147 | SHLVKCAEKEKTFCVNGGECFMVKDLSNPS | EGF-like domain of β1 neuregulin 1 |
| | RYLCKCPNEFTGDRCQNYVMASFYKHLGIE | |
| | FME | |
| 148 | SHLVKCAEKEKTFCVNGGECFMVKDLSNPS | EGF-like domain of β2 neuregulin 1 |
| | RYLCKCPNEFTGDRCQNYVMASFYK | |
| 149 | SHLVKCAEKEKTFCVNGGECFMVKDLSNPS | EGF-like domain of β3 (long) |
| | RYLCKCPNEFTGDRCQNYVMASFYSTSTPFL | neuregulin 1 |
| | SLPE | |
| 150 | SHLVKCAEKEKTFCVNGGECFMVKDLSNPS | EGF-like domain of β3 (short) |
| | RYLCKCPNEFTGDRCQNYVMASFYS | neuregulin 1 |
| 151 | SHLVKCAEKEKTFCVNGGECFMVKDLSNPS | EGF-like domain of α neuregulin 1 |
| | RYLCKCQPGFTGARCTENVPMKVQNQEK | |
| 152 | SHLVKCAEKEKTFCVNGGECFMVKDLSNPS | EGF-like domain of α3 neuregulin 1 |
| | RYLCKCQPGFTGARCTENVPMKVQNQES | |
| 153 | SHLVKCAEKEKTFCVNGGECFMVKDLSNPS | EGF-like domain of α1A neuregulin |
| | RYLCKCQPGFTGARCTENVPMKVQNQEKH | 1 |
| | LGIEFIE | |
| 154 | MSERKEGRGKGKGKKKERGSGKKPESAAG | heparin binding domain of |
| | SQSPALPPRLKEMKSQESAAGSK | neuregulin 1 (alternative 1) |
| 155 | SERKEGRGKGKGKKKERGSGKKPESAAGSQ | heparin binding domain of |
| | SPALPPQLKEMKSQESAAGSKLVLRCETSSE | neuregulin 1 (alternative 2) |
| | YSLRFKWFKNGNELNRKNKPQNIKIQKKPG | |
| | KSELRINKASLADSGEYMCKVISKLG | |
| 156 | PQLKEMKSQESAAGSKLVLRCETSSEYSLRF | heparin binding domain of |
| | KWFKNGNELNRKNKPQNIKIQKKPGKSELRI | neuregulin 1 (alternative 3) |
| | NKASLADSGEYMCKVISKLGNDSASANIT | |
| 157 | SERKEGRGKGKGKKKERGSGKKPESAAGSQ | heparin binding domain of |
| | SPALPPQLKEMKSQESAAGSKLVLRCETSSE | neuregulin 1 (alternative 4) |
| | YSLRFKWFKNGNELNRKNKPQNIKIQKKPG | |
| | KSELRINKASLADSGEYMCKVISKLGNDSAS | |
| | ANIT | |
| 158 | VISKLGNDSASANITIVESNEIITGMPASTEGA | glycosylated domain of neuregulin 1 |
| | YVSSESPIRISVSTEGANTSSSTSTSTTGT | |
| 159 | <as outlined in the sequence protocol> | erbB3 receptor |
| 160 | <as outlined in the sequence protocol> | erbB4 receptor |

The inventors of the present invention have found that the entire extracellular domain (ECD) of neuregulin can cross the blood brain barrier (see examples below). A smaller fragment of neuregulin Nrg1β₁ containing only the EGF-like domain (Thr176-Lys246 of SEQ ID NO:1, 8 kDa) was also capable of passing the intact adult blood brain barrier, yet showed a rather unselective interaction with ErbB-receptors.

It was one object of the present invention to provide a recombinant neuregulin polypeptide which effectively passes the blood brain barrier and/or which selectively interacts with the target receptors such as the erbB3 receptor and/or erbB4 receptor without exhibiting undesired mitogenic properties.

Based on in silico modelling, the inventors assessed that the interaction with the target receptors could be improved by selecting shorter neuregulin fragments and also by generating recombinant polypeptides by fusing the EGF-like domain of neuregulin or fragments thereof to optimized heparin-binding domain(s) and/or to polybasic polypeptides capable of interacting with heparin and/or heparan sulphate. Without being bound by theory, an attractive hypothesis is that neuregulin may be concentrated more specifically at synapses through binding to heparin-like glycosaminoglycans in the extracellular matrix. This may reduce off-target effects, e.g. the activation of receptors other than erbB3 and erbB4 by of the EGF-like domain which may induce cell division which is unwanted due to the risk of cancerogenesis.

Thus, the fusion polypeptides of the invention as outlined below provide therapeutic compounds that comprise only a minimal essential system to bind and activate the respective target receptors. At the same time the size of the polypeptides can be reduced, e.g. by using short linker molecules between the domains or by directly linking the domains to each other. Care was taken to optimize the heparin binding domains to make them as short as possible while retaining their heparin-binding function (see e.g. FIGS. 5 and 6 below).

Accordingly, in a further aspect the invention relates to a polypeptide, wherein the polypeptide comprises or consists of an EGF-like domain (EGFLD1) selected from the group consisting of SEQ ID NO: 140-146 (i.e. SEQ ID NO: 140, 141, 142, 143, 144, 145 and 146), wherein said EGF-like domain may comprise up to one, two, three, four or up to five single amino acid deletions, insertions and/or mutations and wherein said EGF-like domain optionally comprises up to 5, 10, 15, 20, 25, 30, 35 or up to 40 and most preferably up to 30 additional amino acids at its C- and/or N-terminus.

In one embodiment the invention relates to a polypeptide, wherein the polypeptide comprises or consists of an EGF-like domain (EGFLD1) according to SEQ ID NO: 147, wherein said EGF-like domain may comprise up to one, two, three, four, five, six, seven, eight, nine, ten, eleven, twelve or up to thirteen single amino acid deletions, insertions and/or mutations and wherein said EGF-like domain optionally comprises up to 5, 10, 15, 20, 25, 30, 35 or up to 40 and most preferably up to 30 additional amino acids at its C- and/or N-terminus.

In the context of EGF-like domains that are comprised in the polypeptides of the invention, it is preferred that said single amino acid deletion(s) and/or mutation(s) that may be present are not at any of the following positions of said first and, if present, further EGF-like domains, i.e. of SEQ ID NO: 140-146 (i.e. SEQ ID NO: 140, 141, 142, 143, 144, 145 and 146): position 1 (cystein), position 5 (phenylanlanine), position 6 (threonine), position 7 (glycine), position 9 (arginine), position 10 (cystein) and/or position 14 (valine) In other words, it is preferred that the amino acids at position 1, 5, 6, 7, 9, 10 and/or 14 are as specified in SEQ ID NO: 140-146 (i.e. SEQ ID NO: 140, 141, 142, 143, 144, 145 and 146) and are not mutated, deleted or shifted by insertion. Each position is counted from the N-terminus of the sequence according to any of SEQ ID NO: 140-146 (i.e. SEQ ID NO: 140, 141, 142, 143, 144, 145 and 146), as is usual practice in the field. For example, the first position (position 1) refers to the first amino acid in SEQ ID NO 140-146 (i.e. SEQ ID NO: 140, 141, 142, 143, 144, 145 and 146) which is a cystein.

Preferably, the EGF-like domain (EGFLD1) is selected from the group consisting of SEQ ID NO: 147-153 (i.e. SEQ ID NO: 147, 148, 149, 150, 151, 152 and 153) and wherein said EGF-like domain may in total comprise up to one, two, three, four, five, six, seven, eight, nine, ten, eleven, twelve or up to thirteen single amino acid deletions, insertions and/or mutations. In a further preferred embodiment of the aforementioned aspect and preferred embodiment, the EGF-like domain (EGFLD1) is selected from the group consisting of SEQ ID NO: 140-143 (i.e. SEQ ID NO: 140, 141, 142 or 143) or SEQ ID NO: 147-150 (i.e. SEQ ID NO: 147, 148, 149 or 150), i.e. comprises a neuregulin 1-beta EGF-like domain.

Providing a polypeptide that comprises two or more EGF-like domains has been predicted by the inventors to improve the receptor binding ability of the polypeptide of the invention.

Thus, in a further preferred embodiment the polypeptide of the invention further comprises at least one additional EGF-like domain, wherein each additional EGF-like domain is independently selected from the group consisting of SEQ ID NO: 140-153 (i.e. SEQ ID NO: 140, 141, 142, 143, 144, 145, 146, 147, 148, 149, 150, 151, 152 and 153), wherein each additional EGF-like domain may comprise up to one, two, three, four, five, six, seven, eight, nine, ten, eleven, twelve or up to thirteen single amino acid deletions, insertions and/or mutations. In a more preferred embodiment, all EGF-like domains comprised in the polypeptide of the invention in total do not comprise more than five, four, three, two or more than one single amino acid deletions, insertions or mutation.

Thus, the polypeptide of the invention comprises in one embodiment at least a second EGF-like domain (EGFLD2) selected (independently from any other EGF-like domain that may be present) from the group consisting of SEQ ID NO: 140-153 (i.e. SEQ ID NO: 140, 141, 142, 143, 144, 145, 146, 147, 148, 149, 150, 151, 152 and 153), wherein the second EGF-like domain may comprise up to one, two, three, four, five, six, seven, eight, nine, ten, eleven, twelve or up to thirteen single amino acid deletions, insertions and/or mutations.

In a more preferred embodiment, the polypeptide of the invention comprises a first EGF-like domain (EGFLD1) according to SEQ ID NO: 147 and a second EGF-like domain (EGFLD2) according to SEQ ID NO: 147, wherein the first and second EGF-like domain may together comprise up to one, two, three, four, five, six, seven, eight, nine, ten, eleven, twelve or up to thirteen single amino acid deletions, insertions and/or mutations.

Also preferred is the polypeptide of the invention, wherein the polypeptide further comprises a heparin binding domain (HBD). By in silico computational analysis minimal essential heparin binding domains have been identified that based on their charge profile and preferably the presence of an Ig-like domain are expected to improve the specificity of binding of the polypeptides of the invention, thereby reducing any mitogenic effect that the polypeptide may have. Thus, in a preferred embodiment the heparin binding domain of the polypeptide according to the invention has an amino acid sequence according to any of SEQ ID NO: 154, 155, 156 or 157 (most preferably 157) and wherein the heparin binding domain may comprise up to one, two, three, four, five, six, seven, eight, nine, ten, eleven or up to twelve (most preferably up to five) single amino acid deletions, insertions and/or mutations. If the heparin binding domain has one or more single amino acid deletions, insertions and/or mutations it is preferred that between 5% and 40%, more preferably between 15% and 35% and most preferably between 20% and 30% of all amino acids of the heparin binding domain are one or more of the following amino acids: histidine, arginine and lysine.

A “heparin binding domain” as used herein is capable of binding to heparin and/or to heparan sulphate. Heparin is synthesized in cells as a proteoglycan (PG) having in one embodiment a molecular weight of at least 10⁶ Daltons. Heparin is a repeating linear copolymer of 1→4 linked uronic acid and glucosamine residues. Heparan sulfate is a member of the glycosaminoglycan family of carbohydrates and is very closely related in structure to heparin. The most common disaccharide unit within heparan sulfate is composed of a glucuronic acid (G1cA) linked to N-acetylglucosamine (G1cNAc) typically making up around 50% of the total disaccharide units.

Various heparin and heparan sulphate binding proteins are known to the average skilled person and their binding domains are well characterized (see e.g. Hileman, “Glycosaminoglycan-protein interactions: definition of consensus sites in glycosaminoglycan binding proteins”, BioEssays 20:156-167, 1998 John Wiley & Sons, Inc.). In one embodiment, the heparin binding domain comprised in a preferred polypeptide of the invention is an Ig-like (Ig-L) domain that binds to constituents of the extracellular matrix such as heparin (see e.g. Loeb, J. A. & Fischbach, G. D. (1995) J. Cell Biol. 130, 127-135.). One preferred heparin binding domain of the invention is an immunoglobulin-like (Ig-like) domain and most preferably a C2-type immunoglobulin-like domain.

In a more preferred embodiment of the polypeptide of the invention, the polypeptide comprises a heparin binding domain (HBD) having an amino acid sequence according to any of SEQ ID NO: 154, 155, 156 or 157 (most preferably 157) linked to an EGF-like domain (EGFLD1) selected from the group consisting of SEQ ID NO: 140-146 (i.e. SEQ ID NO: 140, 141, 142, 143, 144, 145 and 146) via a linker, wherein the EGF-like domain and said heparin binding domain together may in total comprise up to one, two, three, four, five, six, seven, eight, nine, ten, eleven, twelve or thirteen single amino acid deletions, insertions and/or mutations. The linker is preferably selected from a covalent bond, a chemical linker as described herein and a polypeptide of between 1 and 45 amino acids more preferably of between 1 and 25 amino acids and most preferably of between 1 and 10 amino acids.

In this embodiment it is preferred that between 20% and 50%, more preferably between 20% and 35% and most preferably between 23% and 35% of all amino acids of the heparin binding domain and/or linker are one or more of the following amino acids: histidine, arginine and lysine.

In another embodiment it is preferred that at least 20%, 21%, 22%, 23%, 24%, 25%, 26%, 27%, 28% or at least 29% of all amino acids of the heparin binding domain and/or linker are amino acids selected from the group consisting of histidine, arginine and lysine.

Further preferred embodiments of the polypeptide of the invention comprises or consists of a linker, a HBD and an EGFLD1, wherein the linker is a peptide, linking the HBD as defined herein to the EGFLD 1 as defined herein, wherein the polypeptide further has the features as listed in the table below:

| Total number of single | | |
|---|---|---|
| amino acid deletions, | Linker size | Content of His, Arg and |
| insertions and/or | (length in | Lys in HBD over the |
| mutations in HBD and | number of | entire length of the |
| EGFLD1 together: | amino acids) | HBD |
| between 0 and 10 | between 0 and 45 | between 20% and 35% |
| between 0 and 10 | between 0 and 45 | between 23% and 30% |
| between 0 and 10 | between 0 and 10 | between 20% and 35% |
| between 0 and 10 | between 0 and 10 | between 23% and 30% |
| between 0 and 3 | between 0 and 45 | between 20% and 35% |
| between 0 and 3 | between 0 and 45 | between 23% and 30% |
| between 0 and 3 | between 0 and 10 | between 20% and 35% |
| between 0 and 3 | between 0 and 10 | between 23% and 30% |

Further preferred embodiments of the polypeptide of the invention comprises or consists of a linker, a HBD and an EGFLD1, wherein said linker is a chemical linker or a polypeptide of between 0 and 25 amino acids, linking the HBD as shown to the EGFLD 1 as defined herein, wherein the polypeptide further has the features as listed in the table below:

| | Total number of single | |
|---|---|---|
| | amino acid deletions, | Content of His, Arg |
| | insertions and/or | and Lys in HBD over |
| SEQ ID NO | mutations in HBD and | the entire length of |
| of the HBD | EGFLD1 together: | the HBD |
| 154 | between 0 and 10 | between 20% and 35% |
| 154 | between 0 and 10 | between 23% and 30% |
| 154 | between 0 and 3 | between 20% and 35% |
| 154 | between 0 and 3 | between 23% and 30% |
| 155 | between 0 and 10 | between 20% and 35% |
| 155 | between 0 and 10 | between 23% and 30% |
| 155 | between 0 and 3 | between 20% and 35% |
| 155 | between 0 and 3 | between 23% and 30% |
| 156 | between 0 and 10 | between 20% and 35% |
| 156 | between 0 and 10 | between 23% and 30% |
| 156 | between 0 and 3 | between 20% and 35% |
| 156 | between 0 and 3 | between 23% and 30% |
| 157 | between 0 and 10 | between 20% and 35% |
| 157 | between 0 and 10 | between 23% and 30% |
| 157 | between 0 and 3 | between 20% and 35% |
| 157 | between 0 and 3 | between 23% and 30% |

Further preferred embodiments of the polypeptide of the invention comprises or consists of a linker, a HBD and an EGFLD1, wherein the linker is a chemical linker or a polypeptide of between 0 and 25 amino acids, linking the HBD as shown to the EGFLD1 according to SEQ ID NO: 147, wherein the polypeptide further has the features as listed in the table below:

| | Total number of single amino | Content of His, Arg and |
|---|---|---|
| | acid deletions, insertions and/or | Lys in HBD over the |
| SEQ ID NO | mutations in HBD and EGFLD1 | entire length of the |
| of the HBD | together: | HBD |
| 154 | between 0 and 10 | between 20% and 35% |
| 154 | between 0 and 10 | between 23% and 30% |
| 154 | between 0 and 3 | between 20% and 35% |
| 154 | between 0 and 3 | between 23% and 30% |
| 155 | between 0 and 10 | between 20% and 35% |
| 155 | between 0 and 10 | between 23% and 30% |
| 155 | between 0 and 3 | between 20% and 35% |
| 155 | between 0 and 3 | between 23% and 30% |
| 156 | between 0 and 10 | between 20% and 35% |
| 156 | between 0 and 10 | between 23% and 30% |
| 156 | between 0 and 3 | between 20% and 35% |
| 156 | between 0 and 3 | between 23% and 30% |
| 157 | between 0 and 10 | between 20% and 35% |
| 157 | between 0 and 10 | between 23% and 30% |
| 157 | between 0 and 3 | between 20% and 35% |
| 157 | between 0 and 3 | between 23% and 30% |

Further preferred embodiments of the polypeptide of the invention comprises or consists of a linker, a HBD and an EGFLD1, wherein the linker is a chemical linker or a polypeptide of between 0 and 5 amino acids, linking the HBD as shown to the EGFLD1 according to SEQ ID NO: 147, wherein the polypeptide further has the features as listed in the table below:

| | Total number of | |
|---|---|---|
| | single amino acid | |
| | deletions, insertions | Content of His, Arg |
| | and/or mutations | and Lys in HBD |
| SEQ ID NO | in HBD and | over the entire |
| of the HBD | EGFLD1 together: | length of the HBD |
| 154 | between 0 and 10 | between 20% and 35% |
| 154 | between 0 and 10 | between 23% and 30% |
| 154 | between 0 and 3 | between 20% and 35% |
| 154 | between 0 and 3 | between 23% and 30% |
| 155 | between 0 and 10 | between 20% and 35% |
| 155 | between 0 and 10 | between 23% and 30% |
| 155 | between 0 and 3 | between 20% and 35% |
| 155 | between 0 and 3 | between 23% and 30% |
| 156 | between 0 and 10 | between 20% and 35% |
| 156 | between 0 and 10 | between 23% and 30% |
| 156 | between 0 and 3 | between 20% and 35% |
| 156 | between 0 and 3 | between 23% and 30% |
| 157 | between 0 and 10 | between 20% and 35% |
| 157 | between 0 and 10 | between 23% and 30% |
| 157 | between 0 and 3 | between 20% and 35% |
| 157 | between 0 and 3 | between 23% and 30% |

Further preferred embodiments of the polypeptide of the invention comprises or consists of a linker, a HBD and an EGFLD1, wherein the linker is a chemical linker or a polypeptide of between 0 and 5 amino acids, linking the HBD as shown to the EGFLD1 according to SEQ ID NO: 140, wherein the polypeptide further has the features as listed in the table below:

| | Total number of | |
|---|---|---|
| | single amino acid | |
| | deletions, insertions | Content of His, Arg |
| | and/or mutations | and Lys in HBD over |
| SEQ ID NO | in HBD and | the entire length of the |
| of the HBD | EGFLD1 together: | HBD |
| 154 | between 0 and 10 | between 20% and 35% |
| 154 | between 0 and 10 | between 23% and 30% |
| 154 | between 0 and 3 | between 20% and 35% |
| 154 | between 0 and 3 | between 23% and 30% |
| 155 | between 0 and 10 | between 20% and 35% |
| 155 | between 0 and 10 | between 23% and 30% |
| 155 | between 0 and 3 | between 20% and 35% |
| 155 | between 0 and 3 | between 23% and 30% |
| 156 | between 0 and 10 | between 20% and 35% |
| 156 | between 0 and 10 | between 23% and 30% |
| 156 | between 0 and 3 | between 20% and 35% |
| 156 | between 0 and 3 | between 23% and 30% |
| 157 | between 0 and 10 | between 20% and 35% |
| 157 | between 0 and 10 | between 23% and 30% |
| 157 | between 0 and 3 | between 20% and 35% |
| 157 | between 0 and 3 | between 23% and 30% |

Also preferred is a polypeptide of the invention, which has at least two EGF-like domains and a heparin binding domain, preferably a heparin domain as outlined in above tables. A polypeptide according to this embodiment may optionally comprise one or two linker, linking said domains to each other in any order. Most preferably the aforementioned embodiment has the following features, wherein each linker has a length independently selected from the range as outlined below:

| Maximum number | | |
|---|---|---|
| of single amino acid | | |
| deletions, insertions | Linker | Content of His, Arg |
| and/or mutations in | size (length | and Lys in HBD over |
| HBD, EGFLD1 and | in number of | the entire length of the |
| EGFLD2 together: | amino acids) | HBD |
| between 0 and 10 | between 0 and 45 | between 20% and 35% |
| between 0 and 10 | between 0 and 45 | between 23% and 30% |
| between 0 and 10 | between 0 and 10 | between 20% and 35% |
| between 0 and 10 | between 0 and 10 | between 23% and 30% |
| between 0 and 3 | between 0 and 45 | between 20% and 35% |
| between 0 and 3 | between 0 and 45 | between 23% and 30% |
| between 0 and 3 | between 0 and 10 | between 20% and 35% |
| between 0 and 3 | between 0 and 10 | between 23% and 30% |

In a further preferred embodiment of the polypeptide of the invention, the polypeptide comprises a heparin binding domain (HBD) having an amino acid sequence according to any of SEQ ID NO: 154, 155, 156 or 157 (most preferably 157) linked to an EGF-like domain (EGFLD1) according to SEQ ID NO: 147 via a linker, wherein the EGF-like domain and said heparin binding domain together may in total comprise up to one, two, three, four, five, six, seven, eight, nine, ten, eleven, twelve or thirteen single amino acid deletions, insertions and/or mutations. The linker is preferably selected selected from a covalent bond, a chemical linker as described herein and a polypeptide of between 1 and 45 amino acids more preferably of between 1 and 25 amino acids and most preferably of between 1 and 10 amino acids.

In this embodiment it is preferred that at least 20%, 22%, 24%, 26%, 28%, or at least 29% of all amino acids of the heparin binding domain and/or linker are amino acids selected from the group consisting of histidine, arginine and lysine.

In a further preferred embodiment of the polypeptide of the invention said heparin binding domain is at the N-terminus or the C-terminus of the EGF-like domain and most preferably at the N-terminus.

A further preferred embodiment is a polypeptide according to the invention, wherein the polypeptide further comprises a linker between the EGF-like domain EGFLD 1 and the second EGF-like domain EGFLD2, between any two or more neighbouring EGF-like domains, between said heparin binding domain and said EGF-like domain EGFLD 1 and/or between said heparin binding domain and said second EGF-like domain EGFLD2.

Preferably, the polypeptide according to the invention has a structure selected from:

EGFLD1-linker-EGFLD2,

HBD-linker-EGFLD1-linker-EGFLD2,

EGFLD1-linker-HBD-linker-EGFLD2, or

EGFLD1-linker-EGFLD2-linker-HBD.

As used herein the term “linker” refers to a linker selected from a covalent bond, a chemical linker and a polypeptide, wherein the polypeptide preferably has a length of between 1 and 63 or between 1 and 45 amino acids, more preferably of between 1 and 25 amino acids and most preferably of between 1 and 10 amino acids. If the polypeptide of the invention comprises more than one linker, each liker is independently selected from the group consisting of a covalent bond, a chemical linker and a polypeptide of preferably between 1 and 45 amino acids, more preferably of between 1 and 25 amino acids and most preferably of between 1 and 10 amino acids. If the polypeptide of the invention comprises more than one linker which is a polypeptide, it is understood that the polypeptide for each linker may differ, i.e. is selected independently of other linkers that may be present in the polypeptide of the invention. If said linker is between 1 and 10 amino acids, it is especially preferred that the linker comprises one or more glycine residues, e.g. has the amino acid sequence GGGS. If said linker is a chemical linker it is any chemical group providing a spatial distance between the two entities that are linked via the linker. That distance is preferably sufficient to allow free rotation of the two linked entities. Two polypeptides of the invention can be linked to each other for example by using a divalent aldehyde or using active esters such as disuccinimide esters (e.g. dissuccinimidyl-suberate).

In a preferred embodiment, the linker is a polypeptide having an amino acid sequence according to SEQ ID NO: 158, wherein the linker may comprise up to one, two, three, four, five, six, seven, eight, nine, ten, eleven, twelve, thirteen, fourteen or up to fifteen single amino acid deletions, insertions and/or mutations.

In any embodiment of the polypeptide of the invention where said polypeptide comprises a first EGF-like domain selected from the group consisting of SEQ ID NO: 140-153 (i.e. SEQ ID NO: 140, 141, 142, 143, 144, 145, 146, 147, 148, 149, 150, 151, 152 and 153) (and preferably SEQ ID NO: 147, 148, 149, 150, 151, 152 and 153) (and most preferably SEQ ID NO: 147), a linker according to SEQ ID NO: 158 and a second EGF-like domain independently selected from SEQ ID NO: 140-153 (i.e. SEQ ID NO: 140, 141, 142, 143, 144, 145, 146, 147, 148, 149, 150, 151, 152 and 153) (and preferably SEQ ID NO: 147, 148, 149, 150, 151, 152 and 153) (and most preferably SEQ ID NO: 147), it is preferred that said first EGF-like domain, said linker and said second EGF-like domain in total may comprise up to up to one, two, three, four, five, six, seven, eight, nine, ten, eleven, twelve, thirteen, fourteen or up to fifteen single amino acid deletions, insertions and/or mutations.

In any embodiment of the polypeptide of the invention where said polypeptide comprises an EGF-like domain selected from the group consisting of SEQ ID NO: 140-153 (i.e. SEQ ID NO: 140, 141, 142, 143, 144, 145, 146, 147, 148, 149, 150, 151, 152 and 153) (and preferably SEQ ID NO: 140, 141, 142, 143, 144, 145 and 146), a linker according to SEQ ID NO: 158 and a heparin binding domain having an amino acid sequence according to any of SEQ ID NO: 154, 155, 156 or 157 (most preferably 157), it is preferred that said EGF-like domain, said linker and said heparin-binding domain in total may comprise up to up to one, two, three, four, five, six, seven, eight, nine, ten, eleven, twelve, thirteen, fourteen or up to fifteen single amino acid deletions, insertions and/or mutations and more preferably may comprise up to up to one, two, three, four or up to five single amino acid deletions.

It is preferred that a polypeptide according to the invention specifically binds to the erbB3 receptor (SEQ ID NO: 159) and/or erbB4 receptor (SEQ ID NO: 160). As used herein, a first compound (e.g. a polypeptide or an antibody of the invention) is considered to “specifically bind” to a second compound (e.g. a receptor or an antigen), if it has a dissociation constant K_{D} to said second compound of 100 μM or less, preferably 50 μM or less, preferably 30 μM or less, preferably 20 μM or less, preferably 10 μM or less, preferably 5 μM or less, more preferably 1 μM or less, more preferably 900 nM or less, more preferably 800 nM or less, more preferably 700 nM or less, more preferably 600 nM or less, more preferably 500 nM or less, more preferably 400 nM or less, more preferably 300 nM or less, more preferably 200 nM or less, even more preferably 100 nM or less, even more preferably 90 nM or less, even more preferably 80 nM or less, even more preferably 70 nM or less, even more preferably 60 nM or less, even more preferably 50 nM or less, even more preferably 40 nM or less, even more preferably 30 nM or less, even more preferably 20 nM or less, and even more preferably 10 nM or less and most preferably a K_{D} of less than 1 nM. Methods to determine dissociation constants are well known to the average skilled person such as plasmon resonance, ELISA assays e.t.c,

In one preferred embodiment of the invention the polypeptide of the invention is soluble. The preferred polypeptide is soluble if it is soluble in distilled water to about 1 mg/ml, more preferably to about 10 mg/ml and most preferably to about 20 mg/ml.

In a further preferred embodiment the polypeptide of the inventions is an isolated polypeptide, which is in a further preferred embodiment also soluble as defined above.

It is also preferred that any polypeptide of the invention is able to passes the blood brain barrier, e.g. to cause a therapeutic effect when administered intravenously or via intraperitoneal injection.

Methods to test the permeability of the blood brain barrier have been outlined in the examples below.

As is shown in the examples below, neuregulin polypeptides are therapeutic agents, e.g. useful for the treatment of Parkinson's disease. As the polypeptides of the invention are considered to have improved receptor binding specificity and binding affinity, they can be administered in smaller amounts which reduces the costs of production for a therapeutically effective dosage form and further also minimizes the side-effect for the patient.

Thus, a further aspect of the invention relates to a pharmaceutical composition comprising a polypeptide of the invention.

Preferably, the pharmaceutical composition further comprising a medicament for the treatment of a neurological condition preferably a medicament selected from the group consisting of a compound affecting catecholamine metabolism, an acetylcholine esterase inhibitor, a MAO-B- or COMT- inhibitor, a memantine-type channel blocker, a dopamine or serotonine receptor agonist, a dopamine or serotonine receptor antagonist, a catecholamine or serotonine reuptake inhibitor, an antipsychotic medication, a drug for the treatments of Alzheimer's or Parkinson's disease and a medicament against schizophrenia, bipolar disorder or depression. Preferred medicaments that can be used in this context have already been mentioned above.

Yet another aspect of the invention relates to a polypeptide of the invention for use in the prophylaxis or treatment of a neurological condition. Preferably, said neurological condition is selected from the group of schizophrenia, in particular cognition-related aspects of schizophrenia, bipolar disorder and depression; Parkinson's disease; Alzheimer's disease; epilepsy; MS; ALS; stroke; traumatic brain injury and spinal chord injury. One particularly preferred use is the use to treat bipolar disorder.

Bipolar disorder (BP) is a disabling and often life-threatening disorder that affects approximately 1% of the population worldwide. Bipolar disorder (BP) is characterized by dramatic mood changes, with individuals experiencing alternating episodes of depression and mania interspersed with periods of normal function. BP is chronic, severely disabling, and life-threatening, with increased risk of suicide and estimated lifetime prevalence of ≈1%.

BP has a substantial genetic component. Monozygotic twin concordance rate estimates range from 45 to 70% and sibling recurrence risk estimates from 5 to 10.

Bipolar disorder or manic-depressive disorder, also referred to as bipolar affective disorder or manic depression, is a psychiatric diagnosis that describes a category of mood disorders defined by the presence of one or more episodes of abnormally elevated energy levels, cognition, and mood with or without one or more depressive episodes. In this context, the elevated moods are clinically referred to as mania. In this case one differentiates between unipolar disorder (major depressive disorder) and bipolar disorder.

As was also shown in the examples, peripheral administration of a polypeptide of the invention comprising the ECD of neuregulin resulted in an increase of the total number of dopaminergic tyrosine hydroxylase (TH)+ neurons. Notably, this increase in neurons was not due to cell differentiation, as the Nrg1β1 polypeptide used was shown not to be mitogenic. Since Nrg1β1-ECD did not induce neurogenesis in the adult SNc, the newly appearing dopaminergic neurons apparently resulted from an induction of a dompaminergic phenotype in pre-existing cells. Thus, it was shown that the polypeptides of the invention function to induce cell differentiation.

Accordingly, the invention provides in a further aspect also the use of a polypeptide according to the invention or of a polynucleotide encoding said polypeptide for inducing differentiation of a cell.

As used herein “cell differentiation” or “differentiation of a cell” refers to the alteration of gene expression within a cell upon treating said cell with a differentiation factor such as a polypeptide of the invention. The altered gene expression preferably results in a phenotypic change of the cell, e.g. alteration of size (e.g. volume), shape, membrane potential, metabolic activity and/or responsiveness to signals of said cell. In a preferred embodiment cell differentiation refers to the modulation, preferably induction of a cell's ability of producing dopamine. Thus, in a particularly preferred embodiment of the use of the invention said cell produces more dopamine after having undergone cell differentiation. Most preferably the differentiated cell is a dopaminergic neuron which expresses preferably tyrosine hydroxylase (TH), e.g. can be immunostained for this protein.

In one embodiment said cell to be differentiated is a neuronal cell or a non-neuronal cell, preferably a glial cell. In this context, said neuronal or non-neuronal cell is preferably an erbB4- and/or erbB3-expressing cell. Most preferably said neuronal or non-neuronal cell is an erbB4- and/or erbB3-expressing cell that does not express neuromelanin and/or tyrosine hydroxylase.

The average skilled person can determine without undue burden, whether a cell expresses neuromelanin or tyrosine hydroxylase e.g. by using detectably labelled antibodies which specifically bind neuromelanin and, respectively, tyrosine hydroxylase. Such antibodies can be used e.g. in an ELISA assay to quantify the aforementioned proteins as is well known in the art. A cell is considered to not express neuromelanin or tyrosine hydroxylase if no detectable amount of an antibody that is capable of specifically binding neuromelanin or tyrosine hydroxylase binds to these proteins of said cell as assessed either on a Western blot or in an ELISA assay.

In one embodiment said differentiated cell is characterized by:

(i) a decrease in expression of a protein selected from the group consisting of 14-3-3-zeta (SEQ ID NOs:58, 133), 14-3-3-epsilon (SEQ ID NOs:59, 134), and N-ethylmaleimide sensitive factor (SEQ ID NOs:50, 124) and/or

(ii) an increase in expression of a protein selected from the group consisting of Aldolase A, fructose-bisphosphate (SEQ ID NOs:2, 68); Aldolase C, fructose-bisphosphate (SEQ ID NO:3, 69); Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate-ammonia ligase (Glutamine synthetase) (SEQ ID NOs:10, 78, 79); Dihydrolipoamide S-acetyltransferase (E2 component of pyruvate dehydrogenase complex) (SEQ ID NOs:11, 80, 66); Isocitrate dehydrogenase 3 (NAD+) alpha, isoform CRA_e (SEQ ID NOs:12, 81); Malate dehydrogenase, cytoplasmic (SEQ ID NOs:13, 82); NADH dehydrogenase (ubiquinone) 1 alpha subcomplex, 8 (SEQ ID NOs:14, 83); NADH dehydrogenase (ubiquinone) Fe—S protein 1 (SEQ ID NOs:15, 84, 67); NADH dehydrogenase (ubiquinone) Fe—S protein 8 (SEQ ID NOs:16, 85); Ubiquinol-cytochrome-c reductase complex core protein 1 (SEQ ID NOs:17, 86); ATP synthase, H+ transporting, mitochondrial F0 complex, subunit d (SEQ ID NOs:18, 87, 88); Creatine kinase, brain (SEQ ID NOs:19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+ transporting, lysosomal V1 subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal hydrolase L1, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106); Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110); Neurofilament, light polypeptide (SEQ ID NOs:38, 111); Glial fibrillary acidic protein (SEQ ID NOs:39, 112, 113); Tubulin, alpha 1B (SEQ ID NOs:40, 114); Tubulin, beta (SEQ ID NOs:41, 115); Tubulin, beta 3 (SEQ ID NOs:42, 116); Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1B, member RAS oncogene family; isoform CRA_a (SEQ ID NOs:46, 120); RAB3A, member RAS oncogene family (SEQ ID NOs:47, 121); RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135); Peroxiredoxin 3 (SEQ ID NOs:61, 136); Pyridoxal (pyridoxine, vitamin B6) kinase (SEQ ID NOs:62, 137); and Guanine nucleotide binding protein, alpha o isoform B (SEQ ID NOs:63, 138, 139).

It is to be understood that the above outlined expression changes occur following contacting said cell with a polypeptide of the invention.

In the examples of the present specification it has been shown that the extracellular domain of neuregulin1-β₁ causes cell differentiation and that this domain is not mitogenic. Thus, when using a polypeptide according to the invention or a polynucleotide encoding said polypeptide for inducing differentiation of a cell according to the invention it is preferred that said polypeptide does not induce cell division but only cell differentiation. As the examples have shown this property for the extracellular domain of neuregulin which comprises the EGF-like domain as well as the heparin binding domain, it is preferred that polypeptides of the invention are used which comprise at least one, preferably at least two EGF-like domains and/or heparin-binding domains.

On the basis of the experimental evidence provided in the examples below, it is a further aspect of the invention to provide a method for producing dopaminergic neurons comprising the step a) contacting a non-neuronal cell with a neuregulin isoform of the invention and/or with a polypeptide of the invention.

Preferably said non-neuronal cell used in the method is a non-neuronal cell that does not express neuromelanin or tyrosine hydroxylase such as a cell selected from the group consisting of a glial cell, particularly an astrocyte, oligondentrocyte, an ependymal cell, a radio glial cell, a Schwann cell, a satellite cell and an enteric glia cell.

It has been found that the expression of NRG is increased in patients suffering from a neurodegenerative disease or disorder such as Alzheimer's disease, multiple sclerosis or brain damage (Cannella B, Pitt D, Marchionni M, and Raine C S. Neuregulin and erbB receptor expression in normal and diseased human white matter. J Neuroimmunol 100: 233-242, 1999; Chaudhury A R, Gerecke K M, Wyss J M, Morgan D G, Gordon M N, and Carroll S L. Neuregulin-1 and erbB4 immunoreactivity is associated with neuritic plaques in Alzheimer disease brain and in a transgenic model of Alzheimer disease. J Neuropathol Exp Neurol 62: 42-54, 2003; and Tokita Y, Keino H, Matsui F, Aono S, Ishiguro H, Higashiyama S, and Oohira A. Regulation of Neuregulin Expression in the Injured Rat Brain and Cultured Astrocytes. J Neurosci 21: 1257-1264, 2001.). In line with this, the examples of the present invention show an increase in ErbB4 expression in patients suffering from Parkinson's disease.

Without being bound by theory, the increased expression of neuregulin could present the natural response of the organism to counteract the mentioned diseases. Thus, to boost the natural response, neuregulin isoforms of the invention or polypeptides of the invention can be administered to support the defensive mechanisms of the organism against the respective disease as has been outlined above.

Furthermore, the increased expression of neuregulin and its receptors erbB3 and erbB4 can provide the basis for a diagnostic method wherein the concentration of an endogenous neuregulin (e.g. neuregulin-1 and/or neuregulin-2) is measured as protein or as mRNA and then compared with the concentration found in a healthy subject. If the concentration of neuregulin protein or a polynucleotide encoding neuregulin is found to be increased this is an indication for a disease such as Parkinson's disease, Alzheimer's disease, multiple sclerosis or brain damage.

In the examples it was shown that administration of neuregulin induced a change in expression of a series of proteins in the midbrain (see in particular table 2). Thus, this expression modulation induced by neuregulin will also be diagnostic for the diseases and disorders mentioned above, which also show elevated levels of neuregulin.

Accordingly, another aspect of the invention is an antibody capable of specifically binding to a protein selected from the group consisting of 14-3-3-zeta (SEQ ID NOs:58, 133), 14-3-3-epsilon (SEQ ID NOs:59, 134), N-ethylmaleimide sensitive factor (SEQ ID NOs:50, 124), Aldolase A, fructose-bisphosphate (SEQ ID NOs:2, 68); Aldolase C, fructose-bisphosphate (SEQ ID NO:3, 69); Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate-ammonia ligase (Glutamine synthetase) (SEQ ID NOs:10, 78, 79); Dihydrolipoamide S-acetyltransferase (E2 component of pyruvate dehydrogenase complex) (SEQ ID NOs:11, 80, 66);

Isocitrate dehydrogenase 3 (NAD+) alpha, isoform CRA_e (SEQ ID NOs:12, 81); Malate dehydrogenase, cytoplasmic (SEQ ID NOs:13, 82); NADH dehydrogenase (ubiquinone) 1 alpha subcomplex, 8 (SEQ ID NOs:14, 83); NADH dehydrogenase (ubiquinone) Fe—S protein 1 (SEQ ID NOs:15, 84, 67); NADH dehydrogenase (ubiquinone) Fe—S protein 8 (SEQ ID NOs:16, 85); Ubiquinol-cytochrome-c reductase complex core protein 1 (SEQ ID NOs:17, 86); ATP synthase, H+ transporting, mitochondrial FO complex, subunit d (SEQ ID NOs:18, 87, 88); Creatine kinase, brain (SEQ ID NOs:19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+ transporting, lysosomal V1 subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal terminal hydrolase L1, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106); Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110); Neurofilament, light polypeptide (SEQ ID NOs:38, 111); Glial fibrillary acidic protein (SEQ ID NOs:39, 112, 113); Tubulin, alpha 1B (SEQ ID NOs:40, 114); Tubulin, beta (SEQ ID NOs:41, 115); Tubulin, beta 3 (SEQ ID NOs:42, 116); Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1B, member RAS oncogene family; isoform CRA_a (SEQ ID NOs:46, 120); RAB3A, member RAS oncogene family (SEQ ID NOs:47, 121); RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135); Peroxiredoxin 3 (SEQ ID NOs:61, 136); Pyridoxal (pyridoxine, vitamin B6) kinase (SEQ ID NOs:62, 137); and Guanine nucleotide binding protein, alpha o isoform B (SEQ ID NOs:63, 138, 139)
for use as a diagnostic, preferably as a diagnostic for a disease selected from the group consisting of Alzheimer's disease, multiple sclerosis or brain damage and Parkinsons' disease.

The term “antibody” refers to both monoclonal and polyclonal antibodies, i.e., any immunoglobulin protein or portion thereof which is capable of recognizing an antigen or hapten, i.e., the RNA cap binding domain of PB2 or a peptide thereof. Antigen-binding portions may be produced by recombinant DNA techniques or by enzymatic or chemical cleavage of intact antibodies. In some embodiments, antigen-binding portions include Fab, Fab′, F(ab′)2, Fd, Fv, dAb, and complementarity determining region (CDR) fragments, single-chain antibodies (scFv), chimeric antibodies such as humanized antibodies, diabodies, and polypeptides that contain at least a portion of an antibody that is sufficient to confer specific antigen binding to the polypeptide.

It is well known to the average skilled person of how to raise antibodies against a specific target protein once the sequence of the target protein has been identified.

A further aspect of the invention is a method of diagnosing a disease comprising (i) determining in vitro in an isolated tissue explant or isolated body fluid of a subject the quantity of a protein having at least 90% amino acid sequence identity (preferably over the entire length of the protein selected from the group) with a protein selected from the group consisting of 14-3-3-zeta (SEQ ID NOs:58, 133), 14-3-3-epsilon (SEQ ID NOs:59, 134), N-ethylmaleimide sensitive factor (SEQ ID NOs:50, 124), Aldolase A, fructose-bisphosphate (SEQ ID NOs:2, 68); Aldolase C, fructose-bisphosphate (SEQ ID NO:3, 69); Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate-ammonia ligase (Glutamine synthetase) (SEQ ID NOs:10, 78, 79); Dihydrolipoamide S-acetyltransferase (E2 component of pyruvate dehydrogenase complex) (SEQ ID NOs:11, 80, 66); Isocitrate dehydrogenase 3 (NAD+) alpha, isoform CRA_e (SEQ ID NOs:12, 81); Malate dehydrogenase, cytoplasmic (SEQ ID NOs:13, 82); NADH dehydrogenase (ubiquinone) 1 alpha subcomplex, 8 (SEQ ID NOs:14, 83); NADH dehydrogenase (ubiquinone) Fe—S protein 1 (SEQ ID NOs:15, 84, 67); NADH dehydrogenase (ubiquinone) Fe—S protein 8 (SEQ ID NOs:16, 85); Ubiquinol-cytochrome-c reductase complex core protein 1 (SEQ ID NOs:17, 86); ATP synthase, H+ transporting, mitochondrial F0 complex, subunit d (SEQ ID NOs:18, 87, 88); Creatine kinase, brain (SEQ ID NOs:19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+ transporting, lysosomal V1 subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal hydrolase L1, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106); Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110); Neurofilament, light polypeptide (SEQ ID NOs:38, 111); Glial fibrillary acidic protein (SEQ ID NOs:39, 112, 113); Tubulin, alpha 1B (SEQ ID NOs:40, 114); Tubulin, beta (SEQ ID NOs:41, 115); Tubulin, beta 3 (SEQ ID NOs:42, 116); Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1B, member RAS oncogene family; isoform CRA_a (SEQ ID NOs:46, 120); RAB3A, member RAS oncogene family (SEQ ID NOs:47, 121); RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135); Peroxiredoxin 3 (SEQ ID NOs:61, 136); Pyridoxal (pyridoxine, vitamin B6) kinase (SEQ ID NOs:62, 137); and Guanine nucleotide binding protein, alpha o isoform B (SEQ ID NOs:63, 138, 139) or a polynucleotide encoding said protein;

(ii) optionally determining whether the amount of protein differs from the amount of the same protein quantified in a healthy subject; and

(iii) optionally correlating a change in expression of said protein when compared with the expression of said protein in a healthy subject with a neurological disease which is preferably selected from the group consisting of Alzheimer's disease, multiple sclerosis or brain damage and Parkinsons' disease.

An isolated tissue explant may be any tissue and preferably an isolated brain sample. As used herein “body fluid” is preferably a body fluid selected from the group consisting of cerebrospinal fluid, blood, lymph fluid, saliva and urine.

Multiple methods of quantifying proteins are known to the average skilled person from basic textbooks. Any of these methods can be used in the method of the invention. The subject, which can be a human or non-human patient suffers from a neurological disease selected from the group consisting of Alzheimer' s disease, multiple sclerosis, brain damage or Parkinsons' disease if the expression of the protein or preferably at least three of the above listed proteins deviates by at least 10% from the respective expression of these proteins in a an isolated tissue explant or isolated body fluid of a healthy subject, i.e. a control subject.

In a further aspect the invention also provides a polynucleotide encoding a polypeptide of the invention.

The recombinant soluble neuregulin-1 isoform of the invention or the protein of Table 2 as described herein may be administered according to any route by which effective delivery into the target tissue, e.g. the nervous system, particularly the central nervous system, such as brain and/or spinal chord, is achieved. It was found that pharmaceutically effective concentrations of neuregulin isoforms and fragments thereof may be achieved by systemic administration. For example, the isoforms and polypeptides of the invention may be administered by injection or infusion, e.g. by intravenous injection. Particularly preferred in the context of the present invention is the intraperitoneal administration, e.g, injection. Particularly preferred in the context of the present invention is also the intracerebral administration, e.g., infusion. The isoforms and polypeptides of the invention are preferably administered in an amount of 0.1 to 5000 ng/kg body weight, particularly in an amount of 2 to 1000 ng/kg body weight and more particularly in an amount of 3 to 600 ng/kg body weight of the subject to be treated, depending on the type and severity of the condition to be treated. In other embodiments of the present invention the soluble isoform may also be administered locally, e.g. by direct administration into the central nervous system, e.g. into the spinal chord and/or into the brain. Also administration at higher dosages of up to 500 μg/kg by i.p. or s.c. Injections or infusions, or inhalation devices are may be considered. Preferably the subject to be treated is a mammal, more preferably a human patient.

It is, however, understood that depending on the severity of the disease, the type of the disease, as well as on the respective patient to be treated, e.g. the general health status of the patient, etc., different doses of the pharmaceutical according to the invention are required to elicit a therapeutic effect. The determination of the appropriate dose lies within the discretion of the attending physician.

The soluble recombinant neuregulin-1 isoforms, the protein of Table 2 as described herein and a polypeptide of the invention may be administered as a stand-alone medication, i.e. as a monotherapy or as a co-medication, i.e. in combination with a further agent, particularly with a further agent which is suitable for the treatment of a neurological condition and/or neurological disorder, preferably Parkinson's disease and bipolar disorder. Examples of further agents are compounds affecting catecholamine metabolism, acetylcholine esterase inhibitors, MAO-B- or COMT-inhibitors, Memantine-type channel blockers, dopamine or serotonine receptor agonists or antogonists, catecholamine or serotonine reuptake inhibitors or any type of antipsychotic medicaments like clozapine or olanzapine or gabapentin-like drugs, particularly in the treatment of Alzheimer' s and Parkinson's diseases, schizophrenia, bipolar disorder, depression or other neurological conditions. Additional examples of further agents are neuroprotective agents such as PARP-1 inhibitors, e.g. as disclosed in WO 2006/008118 and WO 2006/008119, which are herein incorporated by reference.

Thus, an embodiment of the present invention refers to the combination of a recombinant soluble neuregulin-1 isoform as described herein, the protein of Table 2 as described herein or a polypeptide of the invention with an agent for the treatment of psychotic disorders such as schizophrenia, bipolar disorders and depression, e.g. olanzapine or clozapine. A further embodiment refers to the combination of a recombinant soluble neuregulin-1 isoform as described herein or a polypeptide of the invention and an agent for the treatment of a neurodegenerative disease such as Parkinson's disease, Alzheimer's disease, MS or ALS. Still a further embodiment refers to the combination of a recombinant soluble neuregulin-1 isoform as described herein or a polypeptide of the invention and an agent for the treatment of epilepsy, neurological injury, such as stroke, traumatic brain injury or spinal chord injury.

The combination therapy may be effected by co-administering the recombinant soluble neuregulin-1 isoform, the protein according to table 2 or the polypeptide of the invention and said further agent in the form of a pharmaceutical composition or kit, wherein the individual agents are administered by separately or via common administration.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following figures and examples are merely illustrative of the present invention and should not be construed to limit the scope of the invention as indicated by the appended claims in any way.

### DESCRIPTION OF THE FIGURES

FIG. 1 ErbB4 expression in dopaminergic neurons of the substantia nigra pars compacta (SNc).

(a,b) Dopaminergic neurons in the human SNc of control persons without neurological disorders (a) and Parkinson's disease (PD) patients (b) were identified by the presence of neuromelanin (NM, brown) in the cytoplasm. The Nrg1β₁-receptor ErbB4 is immunostained in black.

(c-j) Successive magnifications show details from (a) and (b); most NM-containing neurons (brown arrows) show ErbB4 immunoreactivity (black arrows) (g-j), some NM-containing neurons show no ErbB4 immunoreactivity (g,h).

(k) Some neurons in the SNc contain no NM and show strong ErbB4 immunoreactivity in cell bodies (black arrow) and processes (black arrowheads), as demonstrated here in a control person.

(1) Dopaminergic neurons in the mouse SNc were identified by their immunoreactivity for tyrosine hydroxylase (TH, red). ErbB4 is immunostained in green. The merged picture demonstrates that virtually all TH⁺ neurons express ErbB4 (yellow), while some ErbB4 cells in the SNc do not express TH (white arrowheads).

Scale bars (a,b), 250 μm; (c-f), 100 μm; (g- 1), 50 μm.

FIG. 2 Nrg1β₁-ECD passes the blood-brain barrier and phosphorylates ErbB4 in healthy adult mice.

(a) [¹²⁵I]-Nrg1β₁-ECD levels in full blood peaked within 1 hour after a single i.p. injection and remained clearly detectable for at least 12 hours (kCPM=1000 counts per minute).

(b) [¹²⁵I]-Nrg1β₁-ECD penetrated 266.8% more than [¹³¹I]-BSA (bovine serum albumin) into the brain parenchyma, measured 15 minutes after a single i.p. injection. Data are counts per minute in the brain relative to blood; the BSA-uptake was set 100%. *P<0.05, two-sided t-test.

(c-c″) [¹²⁵I]-Nrg1β₁-ECD distribution within the brain parenchyma was studied 1 hour after a single i.p. injection. (c) shows the anatomical map of a sagittal brain section (r=rostral, c=caudal, d=dorsal, v=ventral), stained with cresyl violet (CV, blue); (c′) shows the [¹²⁵I]- Nrg1β₁-ECD autoradiography of the same section (black); (c″) shows superimposed CV (blue) and [¹²⁵I]-Nrg1β₁-ECD (red) images; the strongest [¹²⁵I]-Nrg1β₁-ECD signal was observed in the plexus choroideus of the 4^{th} ventricle (white arrowheads), the lateral ventricle (grey arrowheads) and the tentorium cerebelli (black arrowheads), and particularly strong [¹²⁵I]-Nrg1β₁-ECD signal was obtained in the piriform cortex (light blue arrowheads), the frontal cortex (dark blue arrowheads) and the ventral midbrain containing the substantia nigra (red circle).

(d) ErbB4 receptor was immunoprecipitated from frontal cortex (fCx) and striatum (Str) of mice 1 hour after a single i.p. injection of 10 μg Nrg1β₁-ECD per mouse or vehicle only (NaCl). Probing the eluate with an antibody raised against phosphorylated tyrosine residues (p-Tyr) demonstrated a higher phosphorylation state after Nrg1β₁-ECD-treatment compared to NaCl-treated controls.

(e) Also low doses of Nrg1β₁-ECD (50 ng/kg body weight), administered i.p. once daily on 5 consecutive days, increased ErbB4 phosphorylation in the frontal cortex (fCX), striatum (Str) and SNc 1 hour after the last injection compared to vehicle (NaCl)-injections, as demonstrated by immunohistochemistry with an antibody raised against phosphorylated ErbB4 (p-ErbB4). Quantification of the expression was done by optical density (OD) measurement; OD for NaCl was set 100%. *P<0.05, two-sided t-test.

Scale bars (c-c″), 1 mm; (e), 100 μm.

FIG. 3 Peripherally administered Nrg1β₁-ECD stimulates the nigrostriatal dopaminergic system in healthy adult mice.

(a) Dopamine concentrations in the ventral midbrain (vMes), ventral striatum (vStr) and dorsal striatum (dStr) were significantly elevated 7 days, but not immediately (0 days) after daily i.p. injections of Nrg1β₁-ECD on 5 consecutive days. Values in NaCl-injected controls were set 100%; absolute control values were 0.8±0.1 (vMes), 12.5±2.0 (vStr) and 15.6±3.0 (dStr) ng dopamine per mg wet tissue weight. *P<0.05, ***P<0.001 vs. NaCl-injected controls; ANOVA, post hoc LSD-test.

(b,c) The absolute numbers of dopaminergic tyrosine hydroxylase (TH)⁺ neurons (b) and of large polygonal cresyl violet (CV)⁺ neurons (c) unilaterally in the substantia nigra pars compacta (SNc) were significantly increased 21 days after daily i.p.-injections of Nrg1β₁-ECD on 5 consecutive days. ***P<0.001 vs. NaCl-injected controls; two-sided t-test.

(d) Confocal micrographs of 5-bromo-2′-deoxyuridine (BrdU)⁺ newborn cells (red) and dopaminergic TH⁺ neurons (green) in the SNc of mice 7 days after daily i.p.-injections of NaCl (control) or 21 days after daily i.p.-injections of Nrg1β₁-ECD on 5 consecutive days. The inserts show at higher magnification the localization of BrdU and TH in separate cells.

(e) The absolute numbers of BrdU cells in the unilateral SNc was not significantly altered 7 or 21 days after daily i.p.-injections of Nrg1β₁-ECD on 5 consecutive days, compared to NaCl-injected controls.

(f,g) Differential proteome analysis of the ventral midbrain of mice 7 days after daily i.p.-injections of Nrg1β₁-ECD on 5 consecutive days demonstrated significantly altered levels of N=62 proteins compared to NaCl-treated controls (N=59 upregulated, N=3 downregulated). (f) shows the major functional categories of these proteins; %-values indicate relative numbers of proteins per group. (g,h,i) show the numbers of proteins in the three major functional groups: cytoskeleton (g), energy metabolism (h) and protein quality control (i). Abbreviations: DA, dopamine; IF, intermediate filaments; OxPhos, oxidative phosphorylation; ROS, reactive oxygen species; UPS, ubiquitin-proteasome-system.

Scale bars (d), 100 μm; (d, insert), 10 μm.

FIG. 4 Peripherally administered Nrg1β₁-ECD protects the nigrostriatal dopaminergic system against 6-OHDA-induced toxicity.

Mice received an unilateral striatal 6-OHDA injection or a sham-operation and were treated i.p. with NaCl (Control) or Nrg1β₁-ECD, either instantly (6 hours after 6-OHDA) or with a delay (48 hours after 6-OHDA).

(a) Amphetamine-induced body-turns towards the lesioned side were observed in 6-OHDA-lesioned animals; this pathological asymmetry was prevented, when Nrg1β₁-ECD-treatment was initiated instantly, but not when initiated with delay. n.s., not significant, *P<0.05, **P<0.01, vs. Sham-NaCl; ANOVA, post hoc LSD-test.

(b) Coronal sections of the anterior forebrain of NaCl-treated or instantly Nrg1β₁-ECD-treated mice were immunostained for tyrosine hydroxylase (TH) to visualize dopaminergic fibers in the striatum (left striatum: unlesioned control side; right striatum: sham-operated/6-OHDA-injected side). Note the smaller extent of the 6-OHDA-lesion in the Nrg1β₁-ECD-treated compared to the NaCl-treated mouse.

(c) The optical density of TH⁺ dopaminergic fibers in the striatum of 6-OHDA-lesioned animals was significantly reduced on the lesioned side compared to the unlesioned control side; this pathological asymmetry was significantly attenuated, when Nrg1β₁-ECD-treatment was initiated instantly, but not when initiated with delay. ***P<0.001 vs. Sham-NaCl, ### P<0.001 vs. 6-OHDA-NaCl; ANOVA, post hoc LSD-test.

(d) Coronal sections of the substantia nigra pars compacta of NaCl-treated or instantly Nrg1β₁-ECD-treated mice were TH-immunostained to visualize dopaminergic neurons in the SNc of the sham-operated/6-OHDA-injected side. Note the smaller extent of the 6-OHDA-lesion in the Nrg1β₁-ECD-treated as compared to the NaCl-treated mouse.

(e,f) The number of TH⁺ dopaminergic neurons (e) and cresyl violet⁺ (CV⁺) large neurons (f) in the SNc of 6-OHDA-lesioned animals was significantly reduced on the lesioned side as compared to the unlesioned control side; this pathological asymmetry was significantly attenuated, when Nrg1β₁-ECD-treatment was initiated either instantly or with delay. ***P<0.001 vs. Sham-NaCl, ### P<0.001 vs. 6-OHDA-NaCl; ANOVA, post hoc LSD-test.

(g) All human postmitotic LUHMES neurons in vitro, as identified by immunostaining against the dopamine transporter (DAT, red) surrounding the DAPI⁺ nuclei (blue), expressed the ErbB4 receptor (green) in the cytoplasm. All colors are merged in the last plate.

(h,i) 6-OHDA-induced degeneration of LUHMES cells, as evidenced by a significant increase in LDH release into the culture medium (h) and in the number of pyknotic nuclei per visual field (i); pyknotic DAPI⁺ nuclei were identified as round chromatin clumps of irregular size (i, insert, arrowheads); both phenomena were significantly attenuated by Nrg1β₁-ECD. ***P<0.001 vs. control, ## P<0.01 vs. 6-OHDA; ANOVA, post hoc LSD-test.

Scale bars (b), 2 mm; (d), 200 μm; (g,i), 10 μm.

FIG. 5 Neuregulin ECD charge plot. Shown are charges of the respective amino acids starting from the N-terminus and a polynomial extrapolation of charge over the entire region of the ECD of neuregulin. At the N-terminus there is a region of positive charges which was taken as the basis to optimize the location of the heparin binding domain (HBD)—see also SEQ ID NO:_{—}154-157.

FIG. 6 Preferred recombinant polypeptides of the invention are shown, which are fusion proteins comprising an optimized heparin binding domain (HBD) linked over a short linker (GGGS—which is a preferred linker that can be used with any of the inventive polypeptides described herein) to an EGF-like domain of neuregulin. The fusion proteins comprise a charge-optimized HBD, are shorter than non-modified neuregulin ECD and are therapeutic polypeptides with improved target specificity and reduced mitogenic properties.

### EXAMPLES

### ErbB4 Expression in Human Dopaminergic Neurons is Increased in PD

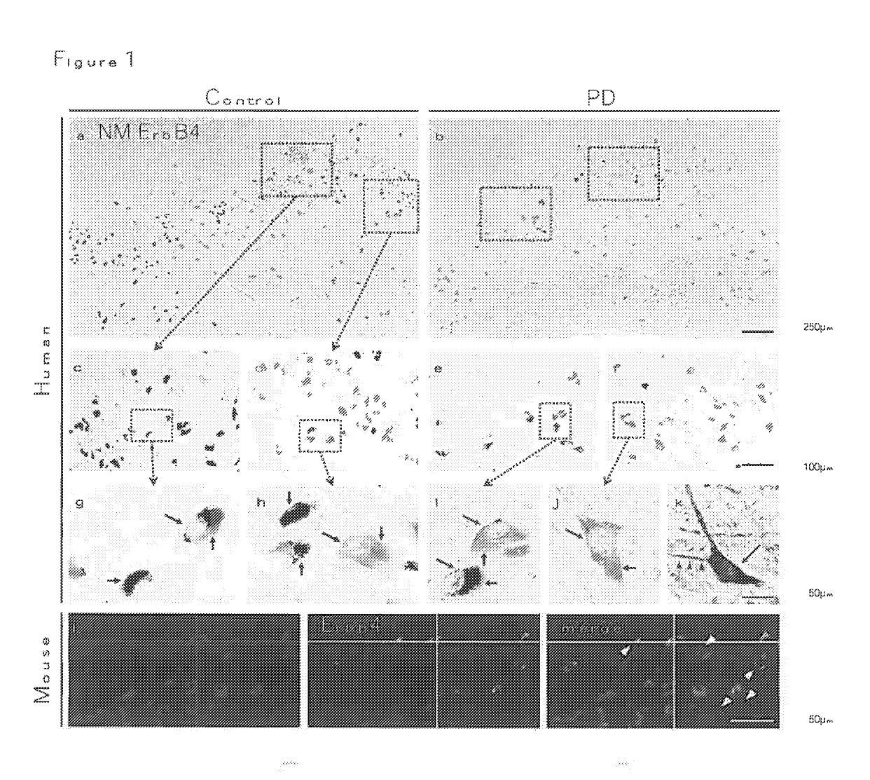

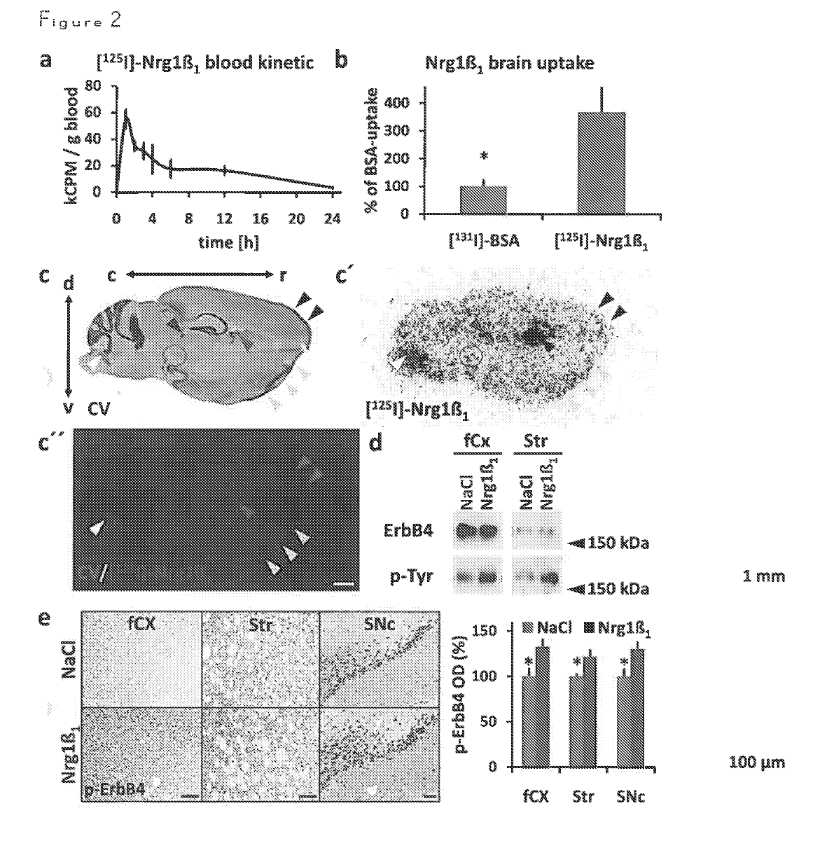

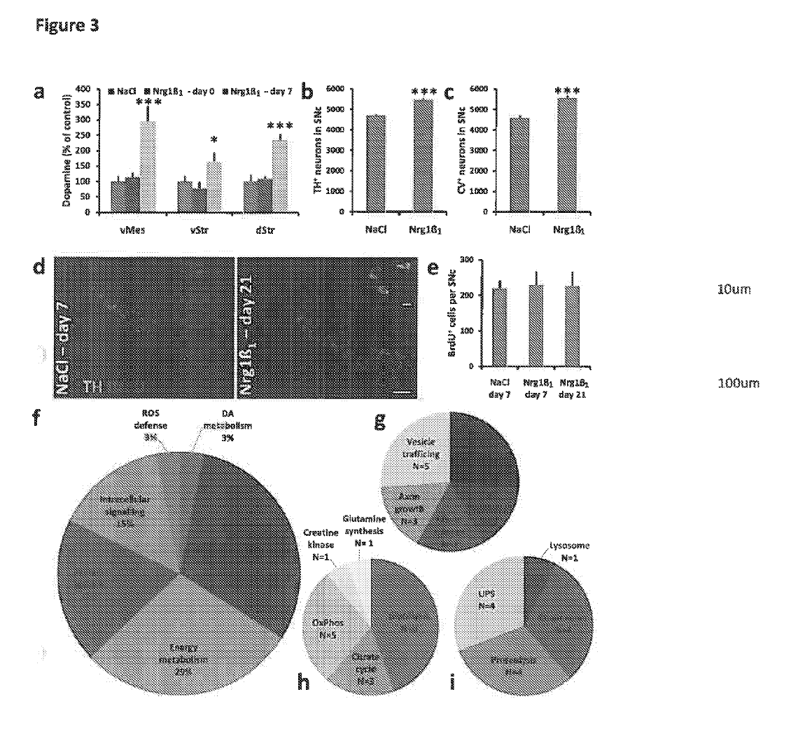

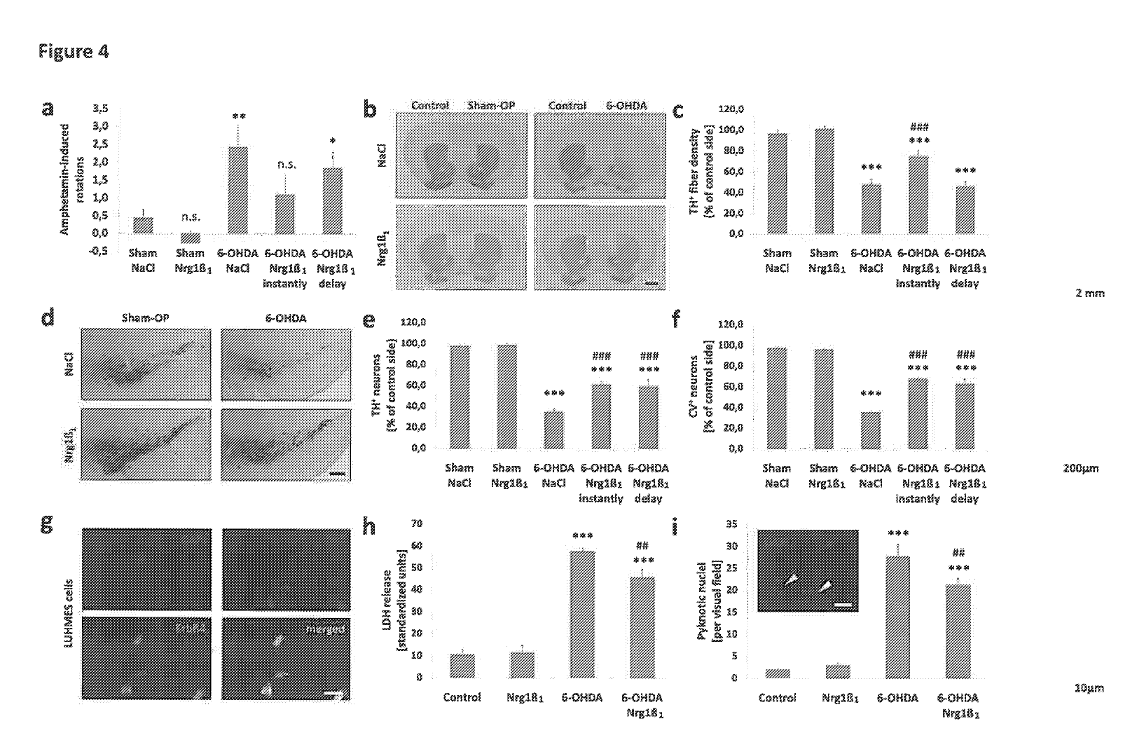

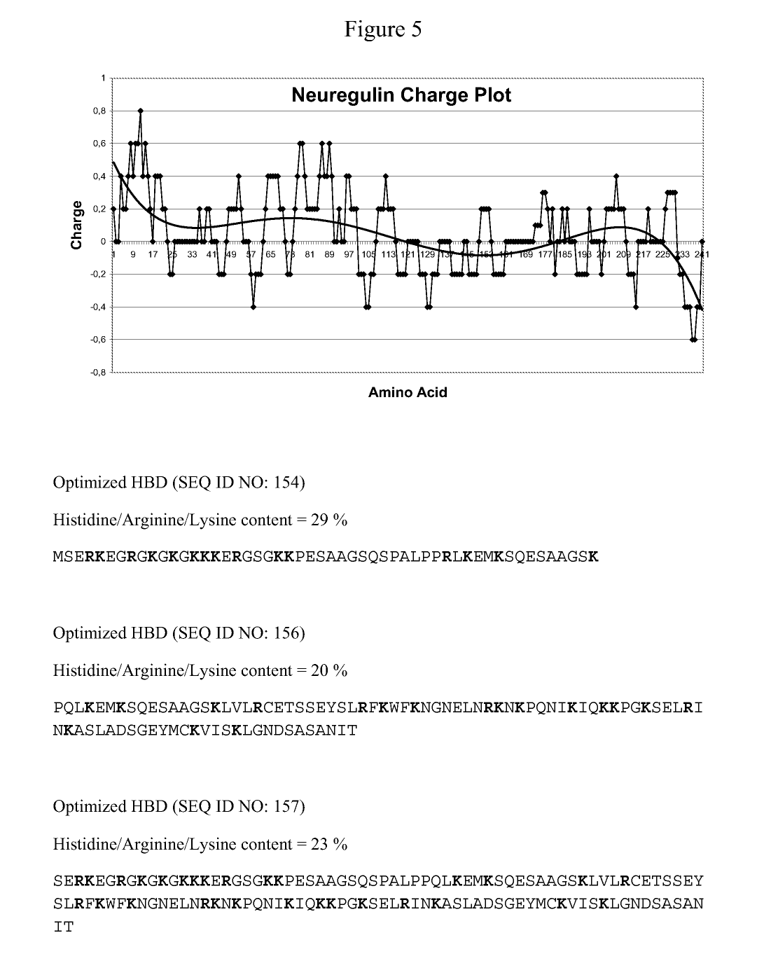

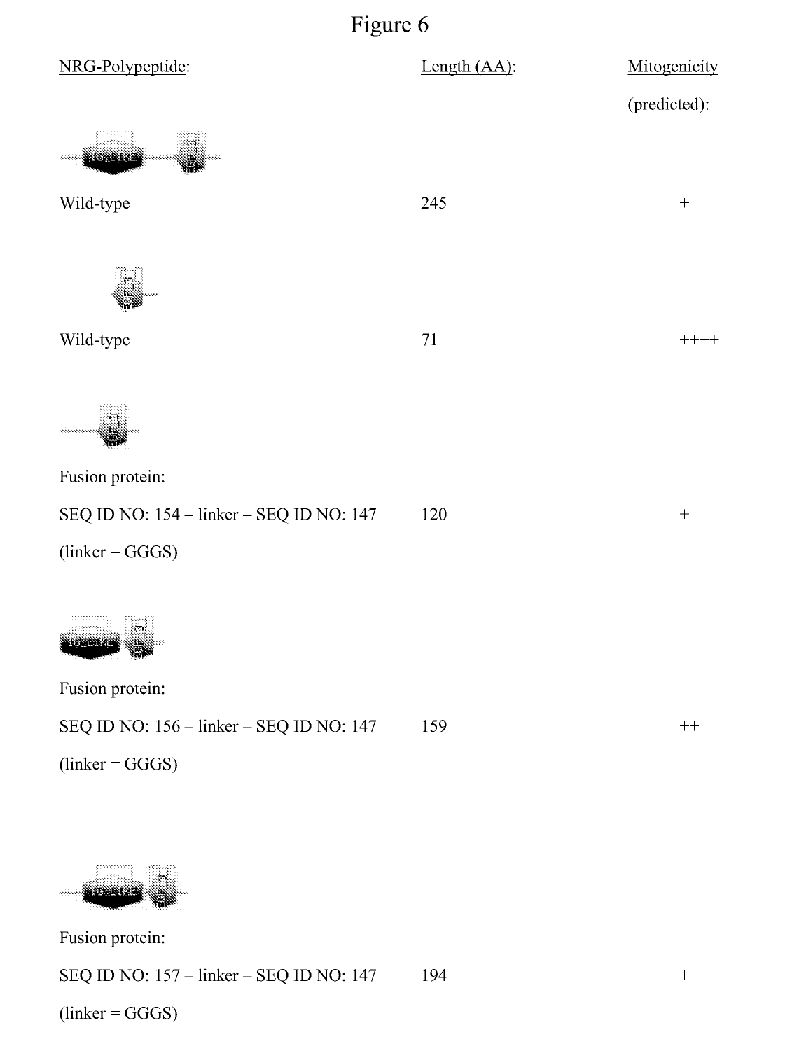

We studied the expression of ErbB4 in dopaminergic neurons, identified by their neuromelanin-content in the substantia nigra pars compacta (SNc) in control persons without neurological disorders and PD patients (FIG. 1a-k).

Most neuromelanin⁺ neurons in the SNc of controls expressed ErbB4. The proportion of neuromelanin⁺ neurons expressing ErbB4 was even higher in PD (Table 1).

Interestingly, there were also some ErbB4⁺ cells in the SNc of controls, which lacked neuromelanin. Their proportion was also increased in PD compared to controls (Table 1).

The predominant ErbB4 expression in dopaminergic neurons and the existence of some ErbB4⁺ cells without dopaminergic phenotype in the SNc was verified in adult mice (FIG. 11).

These observations provide a molecular basis for functional effects of Nrg1β₁ on the nigrostriatal dopaminergic system.

### The Entire ECD of Nrg1β₁ Passes the BBB of Adult Mice

A small fragment of Nrg1β₁ containing only the EGF-like domain (Thr176-Lys246 of SEQ ID NO:1, 8 kDa) passes the intact adult BBB, but interacts rather unselectively with ErbB-receptors. In contrast, the entire ECD of Nrg1β₁ contains an immunoglobulin-like and heparane-sulphate binding motif to target specific neuronal sites, to strengthen specific receptor interactions and thereby to increase the biological activity. Therefore, we worked with a soluble fragment containing the entire ECD of Nrg1β₁ (Nrg1β₁-ECD; Ser2-Lys246 of SEQ ID NO:1; 26.9 kDa; Accession AAA58639). We used human Nrg1β₁-ECD in both mouse and human experimental systems, because of a 97% amino acid sequence homology for ErbB4 between these species.

A single intraperitoneal (i.p.) injection of [¹²⁵I]-Nrg1β₁-ECD led to a peak concentration in blood within 1 hour and remained detectable for at least 12 hours (FIG. 2a). [¹²⁵I]-Nrg1β₁-ECD was found predominantly in the plasma (82.4±6.5%), only 17.6±1.4% were bound to blood cells.

[¹³¹I]-BSA (bovine serum albumin) was used as control protein, which should not readily penetrate the intact BBB. Significantly more [¹²⁵I]-Nrg1β₁-ECD than [¹³¹I]-BSA (+266.8%, P<0.05) was detected in the brain parenchyma at 15 min after a single i.p. injection of both proteins (FIG. 2b), suggesting that the entire 26.9 kD Nrg1β₁-ECD penetrated the intact adult BBB, as shown previously only for the 8 kD EGF-like fragment.

The distribution of peripherally injected [¹²⁵I]-Nrg1β₁-ECD within the brain parenchyma of adult mice, studied by autoradiography 1 hour after a single i.p. injection, showed clear parenchymal signals in the piriform and frontal cortex and particularly in the ventral midbrain containing the SNc (FIG. 2c) compared to [¹²⁵I]-BSA-injected controls, as demonstrated previously only in neonatal mice with a smaller Nrg1β₁-fragment.

### Nrg1β₁-ECD Leads to ErbB4-Phosphorylation in the Adult Mouse Brain

A single i.p. injection of 10 μg Nrg1β₁-ECD led within 1 hour to phosphorylation of ErbB4 in the brains of adult mice, as demonstrated by immunoprecipitation of ErbB4 from the frontal cortex and striatum and probing the eluate with antibodies raised against ErbB4 and phosphorylated tyrosine-residues (FIG. 2d).

Doses as low as 50 ng/kg body weight, administered i.p. once daily on 5 consecutive days, increased ErbB4 phosphorylation in the SNc, as demonstrated by immunohistochemistry with an antibody raised against phosphorylated ErbB4 (FIG. 2e). Therefore, this treatment paradigm was used for the further experiments.

### Nrg1β₁-ECD Increases Dopamine Levels in the Mouse Ventral Midbrain and Striatum

Dopamine concentrations in the ventral midbrain and dorsal striatum (caudate-putamen; see Voorn,P., Vanderschuren,L. J., Groenewegen,H. J., Robbins,T. W., & Pennartz,C. M. Putting a spin on the dorsal-ventral divide of the striatum. Trends Neurosci. 27, 468-474 (2004).) were significantly elevated at day 7, but not immediately (day 0) after daily i.p.-injections of Nrg1β₁-ECD on 5 consecutive days (+194.7% and +136.1%, respectively; P<0.001). The effect in the ventral striatum (nucleus accumbens and olfactory tubercle; see Voorn,P. supra) was less pronounced (+63.8%; P<0.05; FIG. 3a).

### Nrgβ₁-ECD Increases Dopaminergic Cell Numbers in the Normal Mouse SNc

The number of dopaminergic neurons in the SNc, identified by TH-immunostaining, was significantly increased 21 days after daily i.p.-injections of Nrg1β₁-ECD on 5 consecutive days (+16.7%; P<0.001; FIG. 3b).

Also the number of large polygonal cresyl violet-stained neurons with the typical morphology of dopaminergic neurons in the SNc, was increased after Nrg1β₁-ECD-treatment (+21.5%; P<0.001; FIG. 3c).

### Nrg1β₁-ECD is Not Mitogenic in the Normal Adult Mouse SNc

To determine, whether the increase in the number of nigral dopaminergic neurons results from adult neurogenesis, we injected mice once daily with the thymidine analog 5-bromo-2′-deoxyuridine (BrdU) to label mitotic cells, concomitantly with the 5 day Nrg1β₁-ECD-treatment.

We did not find any BrdU and TH co-localization within a single neuron in the SNc at 7 or 21 days after BrdU-injection (FIG. 3d), arguing against Nrg1β₁-induced neurogenesis.

Furthermore, the absolute number of BrdU⁺ cells in the SNc after Nrg1β₁-treatment did not increase compared to NaCl-treated controls (FIG. 3e), indicating that Nrg1β₁ is not mitogenic in the normal adult mouse SNc.

### Nrg1β₁-ECD Induces Proteomic Changes Indicating Neuronal Differentiation in the SNc

The delayed onset of the increase in nigrostriatal dopamine (FIG. 3a) and the increased number of nigral dopaminergic neurons (FIG. 3c,d) in absence of nigral neurogenesis (FIG. 3d,e) suggests that Nrg1β₁-ECD induces neuronal differentiation in the SNc. To approach the nature of this process, we performed a hypothesis-free differential proteome analysis of the ventral midbrain containing the SNc in mice 7 days after 5 consecutive days of Nrg1β₁-ECD-injections compared to NaCl-injections.

N=62 proteins were significantly altered (N=3 were reduced [14-3-3-zeta, 14-3-3-epsilon and N-ethylmaleimide-sensitive factor]; N=59 increased; supplementary Table 2 online). These proteins clustered in six functional groups (FIG. 3f):

1.) Intracellular signaling proteins, including modulators of the ErbB-activated Raf-1 pathway (two 14-3-3 isoforms; mCG7191); phospholipase C, which is also activated downstream of ErbB and increases cytosolic Ca²⁺; and several Ca²⁺-binding and signaling proteins.

2.) Cytoskeletal proteins implicated in actin-, intermediate filament- and microtubule networks, vesicle trafficking and axon outgrowth (FIG. 3g). The protein with the highest increase overall (+2500% vs. NaCl-controls) was dihydropyrimidinase-like 2, also known as collapsin response mediator protein 2, a Ca²⁺-dependent regulator of axonal outgrowth and synaptic plasticity. There was also a 100% increase in RAB3A, known to suppress toxicity in neuronal models of PD (Gitler, A. D.; Bevis, B. J.; Shorter, J.; Strathearn, K. E.; Hamamichi, S.; Su, L. J.; Caldwell, K. A.; Caldwell, G. A.; Rochet, J. C.; McCaffery, J. M.; Barlowe, C.; Lindquist, S.(2008) The Parkinson's disease protein alpha-synuclein disrupts cellular Rab homeostasis PNAS 105, 145-150).

3.) Proteins of dopamine metabolism, namely pyridoxal kinase, an essential cofactor for aromatic-L-amino-acid decarboxylase (AADC) to convert L-dopa into dopamine; and the α-subunit of the Go1α and Go2α GTPases, optimizing vesicular filling of dopamine.

4.) Proteins of energy metabolism including glutamine synthetase, creatine kinase, and several components of glycolysis, citrate cycle and oxidative phosphorylation (complex I [NADH-dehydrogenasd], III [Ubiquinol-reductase] and V [ATP synthase]) (FIG. 3h).

5.) Protein quality control components including chaperones, a lysosomal H⁺-transporting ATPase, proteases and ubiquitin-proteasome-system members, particularly proteasome subunits and the ubiquitin-carboxy-terminal-hydrolase-L1, mutations of which lead familial PD (FIG. 3i). The protein with the second highest increase overall (+2400% vs. NaCl-controls) was valosin-containing protein, a multifunctional protein implicated in ubiquitin-dependent proteolysis, mutations in which cause inclusion-body myopathy and frontotemproal dementia.

6.) Antioxidants, namely peroxiredoxin 1 and 3.

Together, these data suggest that Nrg1β₁-ECD modulates ErbB-downstream and Ca²⁺-dependent signaling cascades, induces neuronal differentiation (axon sprouting, vesicle trafficking, dopamine production and storage) and enhances 1) cellular energy production, 2) defense against oxidative stress and 3) defense against misfolded proteins.

Nrg1β₁-ECD Protects Dopaminergic Mouse Neurons against 6-Hydroxydopamine in vivo

Since the Nrg1β₁-ECD-induced proteomic changes indicate a stimulation of cellular defense systems relevant in the pathophysiology of PD, we investigated, whether Nrg1β₁-ECD can protect dopaminergic neurons in an experimental PD model. We studied 6-hydroxydopamine (6-OHDA)-induced neuronal death, because this neurotoxin potently and irreversibly destroys dopaminergic neurons by 1) reducing ATP-levels, 2) inducing oxidative stress and 3) damaging proteins.

Mice received a unilateral striatal 6-OHDA injection or a sham-operation and were treated i.p. with NaCl (control) or Nrg1β₁-ECD (8×50 ng/kg i.p. in 24 h-intervals). Nrg1β₁-ECD-treatment started either instantly (6 h) after 6-OHDA, when first oxidative stress is generated, or with a delay (48 h), when first, yet partial, axonal and neuronal loss occurs.

Amphetamine-induced body-turns were observed 24 days after 6-OHDA injection as behavioral correlate, indicating unilateral striatal dopamine deficiency on the 6-OHDA-lesioned side. This pathological asymmetry was prevented, when Nrg1β₁-ECD-treatment was initiated instantly, but not when initiated with delay (FIG. 4a).

Histological analysis 28 days after 6-OHDA injection showed consistently a reduced density of TH⁺ dopaminergic fibers in the 6-OHDA-lesioned striatum compared to the unlesioned side. This pathological asymmetry was also attenuated, when Nrg1β₁-ECD-treatment was initiated instantly, but not when initiated with delay (FIG. 4b,c).

The numbers of TH⁺ dopaminergic neurons (FIG. 4d,e) and cresyl violet⁺ large polygonal neurons (FIG. 4f) in the SNc were reduced on the 6-OHDA-lesioned compared to the unlesioned side. This pathological asymmetry, however, was attenuated, when Nrg1β₁-ECD-treatment was initiated either instantly or with delay (FIG. 4e,f).

It is important to notice that the protection of nigral neurons was not a mere consequence of the upregulated cell number observed in healthy controls (FIG. 3b,c), because the cell numbers were calculated as % of the individual animals' contralateral unlesioned SNc.

Nrg1β₁-ECD Protects Human Dopaminergic Neurons Against 6-OHDA in vitro

To verify, whether Nrg1β₁-ECD would also protect human dopaminergic neurons, we used cultures of tetracycline-controlled, v-myc-overexpressing human mesencephalic LUHMES-cells.

Differentiated LUHMES-cells expressed the ErbB4 receptor (FIG. 4g) and were significantly protected in presence of Nrg1β₁-ECD against 6-OHDA-induced degeneration, as studied biochemically using an LDH-release assay (FIG. 4h) and microscopically by counting pyknotic nuclei (FIG. 4i).

### DISCUSSION

We have shown that human Nrg1β₁-ECD is soluble in serum, penetrates into the brain of healthy adult mice, induces phosphorylation of the ErbB4 receptor in the SNc, changes the proteome of the ventral midbrain in a way suggestive of neuronal and dopaminergic differentiation, increases nigrostriatal dopamine levels, activates PD-relevant molecular defense systems, and protects nigrostriatal neurons against the neurotoxin 6-OHDA. We obtained consistent results in the MPTP model (not shown). Since the degenerating dopaminergic neurons in PD strongly express ErbB4, these observations render Nrg1β₁-ECD a promising candidate for symptomatic and neuroprotective therapy of PD patients.

Current therapies of PD are primarily based on dopamine replacement, providing temporary symptomatic improvement of motor symptoms. Unfortunately, patients typically develop drug-induced motor complications (dyskinesia) and no presently available therapy halts the progression of the disease in a clinically relevant manner.

Previous approaches to protect dopaminergic neurons in PD from dying with biological neurotrophic factors were compromised by their proteinaceous nature. The glial cell line-derived neurotrophic factor (GDNF) for example, one of the best studied compounds of this group, potently protects midbrain dopaminergic neurons from a variety of toxic insults (Lin, L. F., Doherty, D. H., Lile, J. D., Bektesh, S., & Collins, F. GDNF: a glial cell line-derived neurotrophic factor for midbrain dopaminergic neurons. Science 260, 1130-1132 (1993)), but does not penetrate the BBB. Thus, several ways to circumvent the BBB were studied (Gill, S. S. Patel, N. K.; Hotton, G. R.; O′Sullivan, K.; McCarter, R.; Bunnage, M.; Brooks, D. J.; Svendsen, C. N.; Heywood, P.Direct brain infusion of glial cell line-derived neurotrophic factor in Parkinson disease. Nat. Med. 9, 589-595 (2003). Kordower, J. H. Emborg, M. E.; Bloch, J.; Ma, S. Y.; Chu, Y.; Leventhal, L.; McBride, J.; Chen, E. Y.; Palfi, S.; Roitberg, B. Z.; Brown, W. D.; Holden, J. E.; Pyzalski, R.; Taylor, M. D.; Carvey, P.; Ling, Z.; Trono, D.; Hantraye, P.; Deglon, N.; Aebischer, P. Neurodegeneration prevented by lentiviral vector delivery of GDNF in primate models of Parkinson's disease. Science 290, 767-773 (2000). Behrstock, S. Behrstock, S.; Ebert, A.; McHugh, J.; Vosberg, S.; Moore, J.; Schneider, B.; Capowski, E.; Hei, D.; Kordower, J.; Aebischer, P.; Svendsen, C. N. Human neural progenitors deliver glial cell line-derived neurotrophic factor to parkinsonian rodents and aged primates. Gene Ther. 13, 379-388 (2006).), but clinical efficacy has not yet been achieved (Lang, A. E. Gill, S.; Patel, N. K.; Lozano, A.; Nutt, J. G.; Penn, R.; Brooks, D. J.; Hotton, G.; Moro, E.; Heywood, P.; Brodsky, M. A.; Burchiel, K.; Kelly, P.; Dalvi, A.; Scott, B.; Stacy, M.; Turner, D.; Wooten, V. G.; Elias, W. J.; Laws, E. R.; Dhawan, V.; Stoessl, A. J.; Matcham, J.; Coffey, R. J.; Traub, M. Randomized controlled trial of intraputamenal glial cell line-derived neurotrophic factor infusion in Parkinson disease. Ann. Neurol. 59, 459-466 (2006)).These constraints apply to other known neurotrophic factors as well (Thoenen, H. & Sendtner, M. Neurotrophins: from enthusiastic expectations through sobering experiences to rational therapeutic approaches. Nat. Neurosci. 5 Suppl, 1046-1050 (2002)).

In contrast, Nrg1β₁-ECD acts physiologically as soluble trophic factor. An N-terminally truncated Nrg1β₁-ECD fragment had already been shown to pass the immature BBB and to phosphorylate ErbB4 in neonatal mice. We have extended these findings by demonstrating that also full length Nrg1β₁-ECD passes the BBB, importantly in adult animals. Upon peripheral administration, we identified radio-labeled Nrg1β₁-ECD in the brain parenchyma. The distribution matched well with the pre-described expression pattern of ErbB4 and ErbB4 receptors (predominantly cerebral cortex and SNc; see Steiner, H., Blum, M., Kitai, S. T., & Fedi, P. Differential expression of ErbB3 and ErbB4 neuregulin receptors in dopamine neurons and forebrain areas of the adult rat. Exp. Neurol. 159, 494-503 (1999)). We also found biochemical and immunohistochemical evidence for phosphorylation of ErbB4 upon peripheral Nrg1β₁-ECD administration. The differential proteome analysis of midbrains from Nrg1β₁-ECD- vs. NaCl-treated mice identified significant changes of phopholipase C and modulators of the Raf-1 pathway, both of which are known downstream signaling components of the ErbB4 receptor. Together, these data support the view that peripherally administered Nrg1β₁-ECD activated cerebral ErbB4 signaling.

In healthy adult mice, Nrg1β₁-ECD increased cerebral dopamine levels. This effect was more pronounced in the dorsal (motor) striatum receiving dopaminergic afferents from the SNc than in the ventral (limbic) striatum receiving dopaminergic afferents from the ventral tegmental area. This is consistent with the previously described higher ErbB4-expression in SNc compared to the ventral tegmental area. The increased dopamine levels were not observed immediately after Nrg1β₁-ECD-treatment, but only after 7 days, suggesting that structural changes rather than acute regulations underlie this phenomenon. Remarkably, Nrg1β₁-ECD also increased the number of phenotypically identified dopaminergic neurons in the SNc. Since Nrg1β₁-ECD did not induce neurogenesis in the adult SNc, the newly appearing dopaminergic neurons apparently resulted from an induction of a dopaminergic phenotype in pre-existing cells, most likely a subpopulation of the ErbB4⁺ cells in the mouse and human SNc, which did not contain TH or neuromelanin. The proteomic analysis also identified an increase in several neuronal cytoskeletal proteins, particularly such implicated in vesicle trafficking and axonal outgrowth, most strikingly collapsin response mediator protein 2. There was a significant upregulation of pyridoxal kinase, an essential cofactor of AADC in dopamine synthesis. Quinoid dihydropteridine reductase, which is part of the pathway to recycle tetrahydrobiopterin, an essential cofactor of TH, was increased by 50% (P=0.09). Finally, the α-subunit of Go-GTPases, optimizing vesicular filling of dopamine, was upregulated. These data shed light on the mechanisms, how Nrg1β₁-ECD structurally strengthens the nigrostriatal dopaminergic system.

Nrg1β₁-ECD also increased numerous proteins implicated in the pathophysiology of PD. Particularly, there was a significant increase in three protein components of complex I of the mitochondrial respiratory chain, which is considered to be dysfunctional in sporadic PD (Mizouno et al., 1989; Mizuno Y, Ohta S, Tanaka M, Takamiya S, Suzuki K, Sato T, Oya H, Ozawa T, Kagawa Y. Deficiencies in complex I subunits of the respiratory chain in Parkinson's disease. Biochem Biophys Res Commun. 1989 Sep 29;163(3):1450-5.) Nrg1β₁-ECD upregulated ubiquitin-carboxy-terminal-hydrolase-L1, responsible for the recycling of ubiquitin, which is dysfunctional in some forms of familial PD³¹.

In addition, Nrg1β₁-ECD increased many other proteins with known functions in the defense against impaired mitochondrial energy production, protein mishandling, oxidative stress and excitotoxicity, which are considered to be the main factors causing neuronal cell death in PD (Dauer, W. & Przedborski, S. Parkinson's disease: mechanisms and models. Neuron 39, 889-909 (2003). Wood-Kaczmar, A.; Gandhi, S.; Wood, N. W. (2006) Understanding the molecular causes of Parkinson's disease. Trends Mol. Med 12, 521-528). Thus, Nrg1β₁-ECD appears to strengthen the midbrain neurons ideally to defend themselves against PD-related stress.

Therefore, we studied, whether Nrg1β₁-ECD can protect dopaminergic neurons against 6-OHDA, a neurotoxin activating all of these pathological mechanisms (Blum, D. Torch, S.; Lambeng, N.; Nissou, M.; Benabid, A. L.; Sadoul, R.; Verna, J. M. Molecular pathways involved in the neurotoxicity of 6-OHDA, dopamine and MPTP: contribution to the apoptotic theory in Parkinson's disease. Prog. Neurobiol. 65, 135-172 (2001)). We used a subacute paradigm with intrastriatal 6-OHDA injection in mice, which allows to separate the temporal sequence of oxidative stress and axonal and neuronal loss (see also Alvarez-Fischer, D. et al. Characterization of the striatal 6-OHDA model of Parkinson's disease in wild type and alpha-synuclein-deleted mice. Exp. Neurol. 210, 182-193 (2008)). Indeed, Nrg1β₁-ECD powerfully protected against 6-OHDA-induced nigral cell loss, striatal axon loss, and corresponding rotational behavior, when administered early after intoxication (i.e. when oxidative stress, but no structural damage was present—see also Alvarez-Fischer et al. supra). If Nrg1β₁-ECD was administered later (i.e. when partial structural damage was already established—see also Alvarez-Fischer et al. supra), the intervention did not protect against striatal axon degeneration and corresponding rotational asymmetry, but still protected nigral neurons from retrograde degeneration.

Previous work has already described neuroprotective effects of glial growth factor-2 (a type II Nrg1)1, 2 on dopaminergic neurons in rat primary midbrain cultures against 6-OHDA (Zhang, L. Fletcher-Turner, A.; Marchionni, M. A.; Apparsundaram, S.; Lundgren, K. H.; Yurek, D. M.; Seroogy, K. B. Neurotrophic and neuroprotective effects of the neuregulin glial growth factor-2 on dopaminergic neurons in rat primary midbrain cultures. J Neurochem. 91, 1358-1368 (2004)). Our finding that Nrg1β₁-ECD (a type I Nrg1)1, 2 protected human dopaminergic LUHMES neurons from 6-OHDA-induced degeneration in vitro suggests that also human midbrain dopaminergic neurons are responsive to Nrg1β₁-ECD. The fact that the vast majority of neuromelanin⁺ dopaminergic neurons in the human SNc express ErbB4 suggests that Nrg1β₁-ECD-treatment might also be effective in living human patients. The observation that a higher proportion of neuromelanin⁺ neurons in the SNc were ErbB4⁺ in PD compared to controls, may indicate that the diseased neurons in PD upregulate ErbB4-expression to seek support. An alternative interpretation may be that the subset of neuromelanin⁺ neurons in the SNc with ErbB4-expression might be less susceptible to degeneration in PD compared to neuromelanin⁺ neurons without ErbB4 expression. However, both interpretations indicate a potential benefit of Nrg1β₁-ECD-treatment in PD.

Nrg1β₁-ECD treatment may have a dual benefit in PD. First, an increase in endogenous dopamine production may provide symptomatic relief and postpone the time until drugs with the risk of inducing motor complications (e.g. L-dopa or dopamine agonists) are required. Second, a protection of the endogenous dopaminergic neurons from degeneration may slow or even halt the progression of the disease.

TABLE 1
ErbB4 expression in the SNc of PD patients and controls.

| | Control | PD | % diff. | P (t-test) |
|---|---|---|---|---|
| N | 5 | 5 | ±0 | n.s. |
| Gender [male:female] | 3:2 | 3:2 | ±0 | n.s. |
| Age [years] | 73.3 ± 4.6 | 73.0 ± 2.3 | −0.4 | n.s. |
| PMD [hours] | 19.0 ± 5.1 | 19.0 ± 5.6 | ±0 | n.s. |
| NM⁺ cells/section | 462 ± 46.6 | 189 ± 42.1 | −59.1 | <0.05 |
| Erbb4⁺ (% of all NM⁺ cells) | 85.0 ± 5.0 | 94.9 ± 2.5 | +11.7 | <0.05 |
| Erbb4⁻ (% of all NM⁺ cells) | 15.0 ± 5.0 | 5.1 ± 1.5 | −66.0 | <0.05 |
| ErbB4+ cells/section | 461.8 ± 56.7 | 250.2 ± 40.9 | −45.8 | <0.05 |
| NM⁻ cells | 14.8 ± 5.1 | 28.7 ± 7.2 | +93.9 | <0.05 |
| (% of all ErbB4⁺ cells) | | | | |

Abbreviations:
N = number;
n.s. = not significant;
PD = Parkinson's disease;
PMD = postmortem delay (i.e. time from death to tissue fixation);
NM = neuromelanin;
% diff. = percent difference in PD relative to Controls.

TABLE 2
online: Nrg1β₁-induced proteome changes in the ventral midbrain.

| Category | Protein | Accession Nr. | % of control | SEM (%) | P-value |
|---|---|---|---|---|---|
| Energy metabolism | | | | | |
| Glycolysis | Aldolase A, fructose-bisphosphate | gi\|6671539 (SEQ ID NOs: 2, 68) | 517.3 | 2.1965 | 0.0292 |
| | Aldolase C, fructose-bisphosphate | gi\|60687506 (SEQ ID NOs: 3, 69) | 179.0 | 14.08 | 0.0249 |
| | Triosephosphate isomerase 1 | gi\|6678413 (SEQ ID NOs: 4, 70, 65) | 164.4 | 13.056 | 0.0335 |
| | Similar to Glyceraldehyde-3-phosphate | gi\|149266302 (SEQ ID NOs: 5, 71, 72) | 387.2 | 20.606 | 0.0049 |
| | dehydrogenaseisoform 1 | | | | |
| | Enolase 1, alpha non-neuron | gi\|34784434 (SEQ ID NOs: 6, 73) | 295.4 | 11.94 | 0.0015 |
| | Enolase 2, gamma neuronal | gi\|7305027 (SEQ ID NOs: 7, 74) | 223.6 | 11.693 | 0.0024 |
| | Lactate dehydrogenase B | gi\|6678674 (SEQ ID NOs: 8, 75) | 189.7 | 11.418 | 0.0064 |
| | Glycerol phosphate dehydrogenase 2, | gi\|123232244 (SEQ ID NOs: 9, 76, 77) | 201.1 | 16.154 | 0.0217 |
| | mitochondrial | | | | |
| Gln synthesis | Glutamate-ammonia ligase (Glutamine | gi\|483918 (SEQ ID NOs: 10, 78, 79) | 236.1 | 17.296 | 0.0212 |
| | synthetase) | | | | |
| Citrate cycle | Dihydrolipoamide S-acetyltransferase | gi\|31542559 (SEQ ID NOs: 11, 80, 66) | 187.0 | 9.7222 | 0.0031 |
| | (E2 component of pyruvate | | | | |
| | dehydrogenase complex) | | | | |
| | Isocitrate dehydrogenase 3 (NAD⁺) | gi\|148693875 (SEQ ID NOs: 12, 81) | 232.8 | 11.38 | 0.0078 |
| | alpha, isoform CRA_e | | | | |
| | Malate dehydrogenase, cytoplasmic | gi\|92087001 (SEQ ID NOs: 13, 82) | 162.8 | 9.8812 | 0.0112 |
| Ox. Phos. | NADH dehydrogenase (ubiquinone) 1 | gi\|21312012 (SEQ ID NOs: 14, 83) | 457.0 | | |
| | alpha subcomplex, 8 | | | | |
| | NADH dehydrogenase (ubiquinone) Fe—S | gi\|21704020 (SEQ ID NOs: 15, 84, 67) | 923.1 | 3.1106 | 0.0348 |
| | protein 1 | | | | |
| | NADH dehydrogenase (ubiquinone) Fe—S | gi\|46195430 (SEQ ID NOs: 16, 85) | 143.0 | 9.6828 | 0.0361 |
| | protein 8 | | | | |
| | Ubiquinol-cytochrome-c reductase | gi\|14548301 (SEQ ID NOs: 17, 86) | 235.6 | 15.815 | 0.0086 |
| | complex core protein 1 | | | | |
| | ATP synthase, H+ transporting, | gi\|21313679 (SEQ ID NOs: 18, 87, 88) | 139.9 | 7.6924 | 0.0184 |
| | mitochondrial F0 complex, subunit d | | | | |
| Creatine | Creatine kinase, brain | gi\|10946574 (SEQ ID NOs: 19, 89) | 191.4 | 11.897 | 0.0074 |
| kinase | | | | | |
| Protein quality control | | | | | |
| Chaperones | Heat shock protein 8 | gi\|42542422 (SEQ ID NOs: 20, 90, 91) | 180.4 | 14.38 | 0.0258 |
| | Heat shock protein 9 | gi\|162461907 (SEQ ID NOs: 21, 92) | 208.4 | 12.564 | 0.0055 |
| | Hsp70 homolog perinuclear form | gi\|435839 (SEQ ID NO: 22) | 208.4 | 12.564 | 0.0055 |
| | (mortalin mot-2) | | | | |
| | Protein disulfide isomerase associated 3 | gi\|112293264 (SEQ ID NOs: 23, 93) | 211.6 | 14.324 | 0.0163 |
| Lysosome | ATPase, H+ transporting, lysosomal V1 | gi\|1184659 (SEQ ID NOs: 24, 94) | 208.4 | 12.564 | 0.0055 |
| | subunit A | | | | |
| UPS | Proteasome 26S subunit, ATPase, 4 | gi\|62201535 (SEQ ID NOs: 25, 55, 96) | 302.7 | 16.015 | 0.0246 |
| | Proteasome subunit alpha type-2 | gi\|1709759 (SEQ ID NOs: 26, 97) | 148.1 | 10.931 | 0.0405 |
| | Ubiquitin carboxy-terminal hydrolase | gi\|148705826 (SEQ ID NOs: 27, 98) | 171.2 | 12.292 | 0.0194 |
| | L1, isoform CRA_b | | | | |
| | Valosin containing protein, isoform | gi\|148670554 (SEQ ID NOs: 28, 99) | 2473.7 | 0.7808 | 0.0076 |
| | CRA_b | | | | |
| Proteolysis | 3-Hydroxyisobutyrate dehydrogenase | gi\|119507488 (SEQ ID NOs: 29, 100) | 183.7 | 12.089 | 0.0107 |
| | Biphenyl hydrolase-like | gi\|21624609 (SEQ ID NOs: 30, 101) | 201.8 | 17.52 | 0.0296 |
| | Haloacid dehalogenase-like hydrolase | gi\|34849757 (SEQ ID NOs: 31, 102) | 145.3 | 9.9981 | 0.0349 |
| | domain containing 2 | | | | |
| Cytoskeleton | | | | | |
| Actin network | Beta-actin (aa 27-375) | gi\|49868 (SEQ ID NOs: 32, 103) | 252.9 | 11.83 | 0.0013 |
| | Gamma-actin | gi\|809561 (SEQ ID NOs: 33, 104) | 178.3 | 8.4756 | 0.0022 |
| | Profilin 2, isoform CRA_b | gi\|148703383 (SEQ ID NOs: 34, 105, 106) | 157.7 | 13.062 | 0.0457 |
| | Transgelin 3 | gi\|9790125 (SEQ ID NOs: 35. 107) | 164.0 | 10.753 | 0.0153 |
| | Annexin A6, isoform CRA_b | gi\|148701560 (SEQ ID NOs: 36, 108, 109) | 180.4 | 14.38 | 0.0258 |
| IF network | Internexin neuronal intermediate | gi\|148539957 (SEQ ID NOs: 37, 110) | 196.6 | 8.6943 | 0.0011 |
| | filament protein. alpha | | | | |
| | Neurofilament, light polypeptide | gi\|39204499 (SEQ ID NOs: 38, 111) | 258.7 | 6.2359 | 0.0014 |
| | Glial fibrillary acidic protein | gi\|14193690 (SEQ ID NOs: 39, 112, 113) | 178.8 | 8.219 | 0.0018 |
| Microtubules | Tubulin, alpha 1B | gi\|34740335 (SEQ ID NOs: 40, 114) | 185.8 | 13.213 | 0.0148 |
| | Tubulin, beta | gi\|21746161 (SEQ ID NOs: 41, 115) | 167.9 | 5.1452 | 0.0006 |
| | Tubulin, beta 3 | gi\|12963615 (SEQ ID NOs: 42, 116) | 185.8 | 13.213 | 0.0148 |
| Axon | Dihydropyrimidinase-like 2 | gi\|40254595 (SEQ ID NOs: 43, 117) | 2607.0 | 1.9289 | 0.0187 |
| sprouting | | | | | |
| | Dihydropyrimidinase-like 4, isoform | gi\|148685897 (SEQ ID NOs: 44, 118) | 506.4 | 6.2263 | 0.0017 |
| | CRA_c | | | | |
| | Brain abundant, membrane attached | gi\|45598372 (SEQ ID NOs: 45, 119) | 178.9 | 9.2437 | 0.0035 |
| | signal protein 1 | | | | |
| Vesicle | RAB1B, member RAS oncogene family, | gi\|148701156 (SEQ ID NOs: 46, 120) | 263.0 | 18.946 | 0.0122 |
| trafficing | isoform CRA_a | | | | |
| | RAB3A, member RAS oncogene family | gi\|6679593 (SEQ ID NOs: 47, 121) | 210.8 | 10.694 | 0.0022 |
| | RAB6A, member RAS oncogene family | gi\|13195674 (SEQ ID NOs: 48, 122) | 173.9 | 14.361 | 0.0326 |
| | Guanosine diphosphate dissociation | gi\|33859560 (SEQ ID NOs: 49, 123) | 256.1 | 13.667 | 0.0230 |
| | inhibitor 1 | | | | |
| | N-ethylmaleimide sensitive fusion | gi\|29789104 (SEQ ID NOs: 50, 124) | 70.0 | 6.9797 | 0.0090 |
| | protein attachment protein beta | | | | |
| Intracellular Signalling | | | | | |
| Calcium | Phospholipase C-alpha | gi\|200397 (SEQ ID NOs: 51, 125) | 211.6 | 14.324 | 0.0163 |
| | Calcineurin B, type I | gi\|149044720 (SEQ ID NOs: 52, 126, 127) | 208.5 | 19.376 | 0.0371 |
| | Calbindin-28K | gi\|6753242 (SEQ ID NOs: 53, 128) | 152.3 | 12.161 | 0.0467 |
| | Calretinin | gi\|393387 (SEQ ID NOs: 54, 129) | 155.6 | 9.5362 | 0.0146 |
| | Visinin-like 1 | gi\|6755983 (SEQ ID NOs: 55, 130) | 185.4 | 10.167 | 0.0042 |
| Chloride | Chloride intracellular channel 4 | gi\|7304963 (SEQ ID NOs: 56, 131) | 126.8 | 3.8203 | 0.0064 |
| | (mitochondrial) | | | | |
| Raf-1 | mCG7191 (Raf Kinase Inhibitor Protein | gi\|148672882 (SEQ ID NOs: 57, 132) | 232.9 | 11.347 | 0.0016 |
| | (RKIP)) | | | | |
| | 14-3-3-zeta | gi\|148676868 (SEQ ID NOs: 58, 133) | 68.3 | 5.8498 | 0.0026 |
| | 14-3-3-epsilon | gi\|148680891 (SEQ ID NOs: 59, 134) | 76.5 | 6.1087 | 0.0178 |
| ROS defense | Peroxiredoxin 1 | gi\|6754976 (SEQ ID NOs: 60, 135) | 185.1 | 17.832 | 0.0498 |
| | Peroxiredoxin 3 | gi\|6680690 (SEQ ID NOs: 61, 136) | 165.2 | 10.361 | 0.0201 |
| DA metabolism | Pyridoxal (pyridoxine, vitamin B6) | gi\|26006861 (SEQ ID NOs: 62, 137) | 195.6 | 10.171 | 0.0028 |
| | kinase | | | | |
| | Guanine nucleotide binding protein, | gi\|164607137 (SEQ ID NOs: 63, 138, 139) | 215.6 | 12.651 | 0.0087 |
| | alpha o isoform B | | | | |

Protein levels in the ventral midbrain of Nrg1β₁-treated mice were expressed as % of control values in NaCl-treated mice.
Gln = glutamine,
Ox. Phos = oxidative phosphorylation;
UPS = ubiquitin-proteasome-system;
IF = intermediate filaments;
ROS = reactive oxygen species;
DA = dopamine.

### METHODS

Human brains

Autopsies from pathologically confirmed PD patients and individuals without neuropsychiatric disorders were obtained from the German Brain Net (www.brain-net.net). Two formalin-fixed, paraffin-embedded, coronal, 7 μm-thick sections containing the SNc were analyzed per brain.

### Animals

The experiments were approved (Regierungspräsidium Giessen; MR20/15-Nr. 84/2007, 29/2008, 68/2009, 73/2009). Male wildtype C57B16 mice (Charles River, Sulzfeld, Germany), 9-11 weeks of age were handled according to the EU Council Directive 86/609/EEC under 12 hour light-dark cycle with food and water ad libitum. Mice were sacrificed with 100 mg/kg pentobarbital i.p. and perfused transcardially with ice-cold 50 mL 0.1 M phosphate buffered saline (PBS).

### Nrg1β₁-ECD

Nrg1β₁-ECD (377-HB/CF; R&D Systems, Minneapolis, Minn.) was dissolved at 10 ng/mL in 0.9% NaCl and injected i.p. (50 ng/kg body weight, unless indicated otherwise). Controls were saline-injected.

### [¹²⁵I]-Nrg1β₁-ECD

Nrg1β₁-ECD and BSA (Fluka, Germany) were iodinated (see e.g. Kastin, A. J., Akerstrom, V., & Pan, W. Neuregulin-1-betal enters brain and spinal cord by receptor-mediated transport. J Neurochem. 88, 965-970 (2004).). 5 μg Nrg1β₁-ECD (0.19 nM) or BSA (74.63 pM) dissolved in 50 μL PBS (0.2 M, pH=7.5) and carrier-free N[¹²⁵I] or N[¹³¹I] (5 μL, 0.24 μCi, Perkin Elmer) were allowed to react (3 hours, room temperature) in a freshly prepared polypropylene iodination vial with 10 μg iodogen (Sigma-Aldrich, Germany). The product was purified by HPLC (C8 column, EC 250/4 Nucleosil 300-5, Macherey-Nagel) with 0.1% trifuoroacetic acid and a gradient of increasing concentrations of 20-60% acetonitrile over 30 minutes, followed by an isocratic elution over 5 minutes and another gradient of linearly increasing concentrations of 60-100% acetonitrile over 5 minutes at a rate of 0.5 mL/min.

[¹²⁵I]-Nrg1β₁-ECD blood Kinetic

N=3 mice were injected i.p. with 1.62 μCi [¹²⁵I]-Nrg1β₁-ECD. Radioactivity was measured in blood with a gamma counter (Cobra II, Perkin-Elmer Packard, Waltham, Mass.).

### [¹²⁵I]-Nrg1β₁-ECD Brain Permeability

Mice were injected i.p. with 1.62 μCi [¹²⁵I]-Nrg1β₁-ECD and [¹³¹I]-BSA (N=5 per group) and sacrificed 1 hour later. Radioactivity of blood and the perfused brains were measured with a gamma counter (Cobra II).

### [¹²⁵I]-Nrg1β₁-ECD Autoradiography

Mice were injected i.p. with 13.51 μCi [¹²⁵I]-Nrg1β₁ or [¹³¹I]-BSA (N=4 per group) and sacrificed and perfused 1 hour later. Sagittal 30 μm microtome brain sections were exposed to a BioMax MS film (Kodak, Stuttgart, Germany) for 30 days.

### ErbB4 Phosphorylation

Mice were injected i.p. with 10 μg Nrg1β₁-ECD or vehicle (N=4 per group) and sacrificed 1 hour later. ErbB4 protein was affinity-purified with a rabbit polyclonal antibody (sc-283, Santa Cruz biotechnology Inc., Heidelberg, Germany) from striatum and frontal cortex. The eluate was subjected to 1D-PAGE and stained with mouse monoclonal antibodies [anti-ErbB4 (sc-8050, Santa Cruz); anti-phospho-Tyrosine (4G10, Millipore, Schwalbach, Germany)].

### HPLC

The ventral midbrain, the ventral striatum co, and the dorsal striatum were dissected, homogenized in 500 μl 0.4 M perchloric acid. Dopamine was measured by reversed phase ion-pair HPLC with electrochemical detection (potential 750 mV) under isocratic conditions using an Ag/AgCl reference electrode.

### BrdU

Mice were injected i.p. with 50 mg BrdU (Sigma; 5 mg/mL in 0.9% NaCl) per kg body weight and 1 hour later with 50 ng Nrg1β₁ per kg body weight and sacrificed 7 or 21 days later (N=5 per group).

### Differential Proteome Analysis

Mice were injected i.p. once daily for 5 consecutive days with 50 ng Nrg1β₁-ECD per kg body weight or 0.9% NaCl and sacrificed 7 days after the last injection (N=5 per group). The ventral midbrain was dissected. Samples were thawed at 25° C., dissolved in 8 M Urea/4% CHAPS/0.1 M Tris (pH 7.4), and iodinated with [¹²⁵I] or [¹³¹I] at identical iodine concentrations. Equal amounts of protein (<5 μg) from a [¹²⁵]- and a [¹³¹I]-labeled sample were mixed and separated by high resolution 2D-PAGE high resolution ‘daisy chains’ covering a pH range of 4-9 (see e.g.

Poznanovic, S., Schwall, G., Zengerling, H., & Cahill, M. A. Isoelectric focusing in serial immobilized pH gradient gels to improve protein separation in proteomic analysis. Electrophoresis 26, 3185-3190 (2005)). Quantitative differential displays of the [¹²⁵I]- and [¹³¹I]-signals of proteins from Nrg1β₁-ECD- and NaCl-samples were generated using a sensitive radio-imaging technique (see e.g. Groebe, K. et al. Differential proteomic profiling of mitochondria from Podospora anserina, rat and human reveals distinct patterns of age-related oxidative changes. Exp. Gerontol. 42, 887-898 (2007)). Protein spots with significantly different intensities between the Nrg1β₁-ECD- and NaCl-groups were determined (GREG software, www.fit.fraunhofer.de), excised from the 2D-PAGE-gels (ProPick robot, Genomic Solutions Ltd., Huntingdon, UK), cleaved by trypsin (ProGest robot, Genomic Solutions Ltd.), and applied onto an anchor target (ProMS robot, Genomic Solutions Ltd.). Mass spectra of peptide ions were obtained with an Ultraflex MALDI time-of-flight (TOF) mass spectrometer (Bruker, Bremen, Germany) in reflector mode within a mass range from m/z 800 to 4000 (see e.g. Groebe, K. et al. supra). Peptide mass fingerprints were searched against the non-redundant NCBI Protein Sequence Database (Mascot server software, Matrix Science, London, UK).

### 6-OHDA In Vivo

6-OHDA (Sigma; 5 μg in 2 μL 0.9% NaCl with 0.2 μg/μL ascorbic acid) was injected slowly (0.5 μL/min) into the striatum (coordinates: AP+0.9 mm, ML −1.8 mm, DV −3.0 mm relative to bregma and dura) of anesthetized mice (1% ketamine/0.2% xylazine, 10 ml/kg body weight); controls were vehicle-injected (sham-OP). Mice received 50 ng Nrg1β₁-ECD per kg body weight once daily at noon for 8 consecutive days starting instantly (6 h) or delayed (48 h) after 6-OHDA injection; controls were saline-injected. Mice were sacrificed 28 days after 6-OHDA injection (N=10-12 per group).

### Amphetamine-Induced Rotation

Four days prior to sacrifice, all mice received 5 mg d-amphetamine (Sigma) per kg body weight i.p. Rotational behavior was monitored for 30 minutes (Viewer II, rotation plug-in, Biobserve, Bonn, Germany). Total net 360° body turns per minute were calculated; positive values indicate rotations ipsilateral to the lesioned side.

### 6-OHDA In Vitro

LUHMES cells were proliferated and differentiated as described (Lotharius, J. Falsig, J.; van,Beek J.; Payne, S.; Dringen, R.; Brundin, P.; Leist, M. Progressive degeneration of human mesencephalic neuron-derived cells triggered by dopamine-dependent oxidative stress is dependent on the mixed-lineage kinase pathway. J. Neurosci. 25, 6329-6342 (2005)). Differentiated cells were treated with or without 100 ng Nrg1β₁ per mL medium and intoxicated 1 hour later for 48 hours with 32 μM 6-OHDA.

### LDH Release

LDH levels in the culture medium were measured from at least 12 wells per condition from three independent experiments using the CytoTox-ONE™ Homogeneous Membrane Integrity Assay (Promega, Mannheim, Germany).

### Immunohistochemistry

Mounted human sections, free-floating mouse sections or cultures were stained with the following antibodies: rabbit polyclonal anti-TH (P40101-0, Pel-Freez Biologicals, Rogers, AR; 1/1000), rat polyclonal anti-DAT (MAB369, Chemicon International, Temecula, Calif.; 1/1000), rat polyclonal anti-BrdU (OBT0030CX, Immunologicals Direct, Oxfordshire, UK; 1/500), rabbit polyclonal anti-ErbB4 (sc-283, Santa Cruz Biotechnology, Santa Cruz, Calif.; 1/200), rabbit polyclonal anti-Y1284-phosphorylated-ErbB4 (ab61059, Abcam, Cambridge, UK; 1/200).

### Image Analysis

All neuromelanin-containing neurons in the human SNc per section were analyzed to determine their number and percentage of ErbB4 expression by brightfield microscopy (DM-RB, Leica, Wetzlar, Germany; SimplePCI 6.1 software; Hamamatsu Photonics, Herrsching, Germany). Double-immunofluorescence was analyzed by confocal microscopy (Leitz TCS SP5, Leica; MetaMorph software, Molecular Devices, Munich, Germany). Unbiased stereological estimations of total cell numbers were determined on every 5^{th} serial section over the entire rostro-caudal extension of the mouse SNc (−2.4 to −4.1 mm from bregma) using the optical fractionator method

(Microphot FX, Olympus, Hamburg, Germany; Stereoinvestigator software, MicroBrightField, Magdeburg, Germany). The optical density of TH⁺ fibers was quantified at 8 equally spaced sections in the striatum (1.7 to −0.5 mm from bregma) under bright-field illumination (eVision Copylizer, Kayser Fototechnik, Buchen, Germany; ImageJ v1.42 software, NIH, Bethesda, Md.) and corrected for background in adjacent white matter. The optical density of p-ErbB4-immunoreactivity was measured identically in anatomically matched sections in frontal cortex (+1.7 mm from bregma), striatum (+1.0 mm) and SNc (−3.0 mm). Pyknotic DAPI⁺ nuclei in cultures, identified as round chromatin clumps of irregular size, were counted in ten randomly distributed visual fields per culture well (DM-IRB; Leica; SimplePCI 6.1, Hamamatsu).

### Statistics

Data are shown as mean ±s.e.m. Normal, parametric data were compared with the two-sided, unpaired t-test or ANOVA followed by post-hoc Fisher least significant difference (LSD) test. P<0.05 was considered significant.

1. A polypeptide, wherein the polypeptide comprises or consists of an EGF-like domain (EGFLD1) selected from the group consisting of SEQ ID NO: 140-146, wherein said EGF-like domain may comprise up to five single amino acid deletions, insertions and/or mutations and wherein said EGF-like domain optionally comprises up to 30 additional amino acids at its C- and/or N-terminus.

2. The polypeptide according to claim 1, wherein the EGF-like domain (EGFLD1) is selected from the group consisting of SEQ ID NO: 147-153 and wherein said EGF-like domain may comprise up to 13 single amino acid deletions, insertions and/or mutations.

3. The polypeptide according to claim 1, wherein the polypeptide further comprises at least one additional EGF-like domain, each independently selected from the group consisting of SEQ ID NO: 140-153, wherein each additional EGF-like domain may comprise up to 13 single amino acid deletions, insertions and/or mutations.

4. The polypeptide according to claim 1, wherein the polypeptide comprises at least a second EGF-like domain (EGFLD2) selected from the group consisting of SEQ ID NO: 140-153, wherein the second EGF-like domain may comprise up to 13 single amino acid deletions, insertions and/or mutations.

5. The polypeptide according to claim 1, wherein the polypeptide further comprises a heparin binding domain (HBD).

6. The polypeptide according to claim 5, wherein the heparin binding domain has an amino acid sequence according to any of SEQ ID NO: 154-157 and wherein the heparin binding domain may comprise up to 12 single amino acid deletions, insertions and/or mutations.

7. The polypeptide according to claim 5, wherein said heparin binding domain is at the N-terminus or the C-terminus of the EGF-like domain.

8. The polypeptide according to claim 4, wherein the polypeptide further comprises a linker between the EGF-like domain EGFLD 1 and the second EGF-like domain EGFLD2, between any two or more neighbouring EGF-like domains, between said heparin binding domain and said EGF-like domain EGFLD 1 and/or between said heparin binding domain and said second EGF-like domain EGFLD2.

9. The polypeptide according to claim 8, wherein the polypeptide has the structure:
EGFLD 1-linker-EGFLD2,
HBD-linker-EGFLD 1-linker-EGFLD2,
EGFLD 1-linker-HBD-linker-EGFLD2, or
EGFLD 1-linker-EGFLD2-linker-HBD.

10. The polypeptide according to claim 8, wherein each linker is individually selected from a covalent bond, a chemical linker and a polypeptide preferably having a length of up to 45 amino acids.

11. The polypeptide according to claim 10, wherein the linker has an amino acid sequence according to SEQ ID NO: 158, wherein the linker may comprise up to fifteen single amino acid deletions, insertions and/or mutations.

12. The polypeptide according to claim 1, wherein the polypeptide specifically binds to the erbB3 receptor (SEQ ID NO: 159) and/or erbB4 receptor (SEQ ID NO: 160).

13. Pharmaceutical composition comprising a polypeptide of claim 1.

14. The pharmaceutical composition according to claim 13, further comprising a medicament for the treatment of a neurological condition preferably a medicament selected from the group consisting of a compound affecting catecholamine metabolism, an acetylcholine esterase inhibitor, a MAO-B- or COMT-inhibitor, a memantine-type channel blocker, a dopamine or serotonine receptor agonist, a dopamine or serotonine receptor antagonist, a catecholamine or serotonine reuptake inhibitor, an antipsychotic medication, a drug for the treatments of Alzheimer's or Parkinson's disease and a medicament against schizophrenia, bipolar disorder or depression.

15. A polypeptide of claim 1 for use in the prophylaxis or treatment of a neurological condition.

16. The polypeptide of claim 15, wherein the neurological condition is selected from the group of schizophrenia, in particular cognition-related aspects of schizophrenia, bipolar disorder and depression; Parkinson's disease; Alzheimer's disease; epilepsy; MS; ALS; stroke; traumatic brain injury and spinal chord injury.

17. Use of a polypeptide according to claim 1 or a polynucleotide encoding said polypeptide for inducing differentiation of a cell.

18. Antibody capable of specifically binding to a protein selected from the group consisting of 14-3-3-zeta (SEQ ID NOs:58, 133), 14-3-3-epsilon (SEQ ID NOs:59, 134), N-ethylmaleimide sensitive factor (SEQ ID NOs:50, 124), Aldolase A, fructose-bisphosphate (SEQ ID NOs:2, 68); Aldolase C, fructose-bisphosphate (SEQ ID NO:3, 69);
Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate- ammonia ligase (Glutamine synthetase) (SEQ ID NOs: 10, 78, 79); Dihydrolipoamide S-acetyltransferase (E2 component of pyruvate dehydrogenase complex) (SEQ ID NOs: II, 80, 66); Isocitrate dehydrogenase 3 (NAD+) alpha, isoform CRA_e (SEQ ID NOs: 12, 81); Malate dehydrogenase, cytoplasmic (SEQ ID NOs: 13, 82); NADH dehydrogenase (ubiquinone) 1 alpha subcomplex, 8 (SEQ ID NOs: 14, 83); NADH dehydrogenase (ubiquinone) Fe—S protein 1 (SEQ ID NOs: 15, 84, 67); NADH dehydrogenase (ubiquinone) Fe—S protein 8 (SEQ ID NOs: 16, 85); Ubiquinol-cytochrome-c reductase complex core protein 1 (SEQ ID NOs: 17, 86); ATP synthase, H+ transporting, mitochondrial F0 complex, subunit d (SEQ ID NOs: 18, 87, 88); Creatine kinase, brain (SEQ ID NOs: 19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+ transporting, lysosomal VI subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal hydrolase LI, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106);
Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110);
Neurofilament, light polypeptide (SEQ ID NOs:38, 111); Glial fibrillary acidic protein (SEQ ID NOs:39, 112, 113); Tubulin, alpha IB (SEQ ID NOs:40, 114); Tubulin, beta (SEQ ID NOs:41, 115); Tubulin, beta 3 (SEQ ID NOs:42, 116);
Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1 B, member RAS oncogene family; isoform CRA_a (SEQ ID NOs:46, 120); RAB3A, member RAS oncogene family (SEQ ID NOs:47, 121);
RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135);
Peroxiredoxin 3 (SEQ ID NOs:61, 136); Pyridoxal (pyridoxine, vitamin B6) kinase (SEQ ID NOs:62, 137); and Guanine nucleotide binding protein, alpha o isoform B (SEQ ID NOs:63, 138, 139)
for use as a diagnostic.

19. Method of diagnosing a disease comprising
(i) determining in vitro in an isolated tissue explant or isolated body fluid of a subject the quantity of a protein having at least 90% amino acid sequence identity with a protein selected from the group consisting of 14-3-3-zeta (SEQ ID NOs:58, 133), 14-3-3-epsilon (SEQ ID NOs:59, 134), N-ethylmaleimide sensitive factor (SEQ ID NOs:50, 124), Aldolase A, fructose-bisphosphate (SEQ ID NOs:2, 68); Aldolase C, fructose-bisphosphate (SEQ ID NO:3, 69); Triosephosphate isomerase 1 (SEQ ID NOs:4, 65, 70); similar to Glyceraldehyde-3-phosphate dehydrogenaseisoform 1 (SEQ ID NOs:5, 71, 72); Enolase 1, alpha non-neuron (SEQ ID NOs:6, 73); Enolase 2, gamma neuronal (SEQ ID NOs:7, 74); Lactate dehydrogenase B (SEQ ID NOs:8, 75); Glycerol phosphate dehydrogenase 2, mitochondrial (SEQ ID NOs:9, 76, 77); Glutamate-ammonia ligase (Glutamine synthetase) (SEQ ID NOs: 10, 78, 79); Dihydrolipoamide S-acetyltransferase (E2 component of pyruvate dehydrogenase complex) (SEQ ID NOs: I I, 80, 66); Isocitrate dehydrogenase 3 (NAD+) alpha, isoform CRA_e (SEQ ID NOs: 12, 81); Malate dehydrogenase, cytoplasmic (SEQ ID NOs: 13, 82); NADH dehydrogenase (ubiquinone) 1 alpha subcomplex, 8 (SEQ ID NOs: 14, 83); NADH dehydrogenase (ubiquinone) Fe—S protein 1 (SEQ ID NOs: 15, 84, 67); NADH dehydrogenase (ubiquinone) Fe—S protein 8 (SEQ ID NOs: 16, 85); Ubiquinol-cytochrome-c reductase complex core protein 1 (SEQ ID NOs: 17, 86); ATP synthase, H+ transporting, mitochondrial F0 complex, subunit d (SEQ ID NOs: 18, 87, 88); Creatine kinase, brain (SEQ ID NOs: 19, 89); Heat shock protein 8 (SEQ ID NOs:20, 90, 91); Heat shock protein 9 (SEQ ID NOs:21, 92); Hsp70 homolog perinuclear form (mortalin mot-2) (SEQ ID NO:22); Protein disulfide isomerase associated 3 (SEQ ID NOs:23, 93); ATPase, H+transporting, lysosomal VI subunit A (SEQ ID NOs:24, 94); Proteasome 26S subunit, ATPase, 4 (SEQ ID NOs:25, 95, 96); Proteasome subunit alpha type-2 (SEQ ID NOs:26, 97); Ubiquitin carboxy-terminal hydrolase LI, isoform CRA_b (SEQ ID NOs:27, 98); Valosin containing protein, isoform CRA_b (SEQ ID NOs:28, 99); 3-Hydroxyisobutyrate dehydrogenase (SEQ ID NOs:29, 100); Biphenyl hydrolase-like (SEQ ID NOs:30, 101); Haloacid dehalogenase-like hydrolase domain containing 2 (SEQ ID NOs:31, 102); Beta-actin (aa 27-375) (SEQ ID NOs:32, 103); Gamma-actin (SEQ ID NOs:33, 104); Profilin 2, isoform CRA_b (SEQ ID NOs:34, 105, 106); Transgelin 3 (SEQ ID NOs:35, 107); Annexin A6, isoform CRA_b (SEQ ID NOs:36, 108, 109); Internexin neuronal intermediate filament protein, alpha (SEQ ID NOs:37, 110); Neurofilament, light polypeptide (SEQ ID NOs:38, 111); Glial fibrillary acidic protein (SEQ ID NOs:39, 112, 113); Tubulin, alpha IB (SEQ ID NOs:40, 114); Tubulin, beta (SEQ ID NOs:41, 115); Tubulin, beta 3 (SEQ ID NOs:42, 116); Dihydropyrimidinase-like 2 (SEQ ID NOs:43, 117); Dihydropyrimidinase-like 4, isoform CRA_c (SEQ ID NOs:44, 118); Brain abundant, membrane attached signal protein 1 (SEQ ID NOs:45, 119); RAB1 B, member RAS oncogene family; isoform CRA_a (SEQ ID NOs:46, 120); RAB3A, member RAS oncogene family (SEQ ID NOs:47, 121); RAB6A, member RAS oncogene family (SEQ ID NOs:48, 122); Guanosine diphosphate dissociation inhibitor 1 (SEQ ID NOs:49, 123); Phospholipase C-alpha (SEQ ID NOs:51, 125); Calcineurin B, type I (SEQ ID NOs:52, 126, 127); Calbindin-28K (SEQ ID NOs:53, 128); Calretinin (SEQ ID NOs:54, 129); Visinin-like 1 (SEQ ID NOs:55, 130); Chloride intracellular channel 4 (mitochondrial) (SEQ ID NOs:56, 131); mCG7191 (Raf Kinase Inhibitor Protein (RKIP)) (SEQ ID NOs:57, 132); Peroxiredoxin 1 (SEQ ID NOs:60, 135);
Peroxiredoxin 3 (SEQ ID NOs:61, 136); Pyridoxal (pyridoxine, vitamin B6) kinase (SEQ ID NOs:62, 137); and Guanine nucleotide binding protein, alpha o isoform B (SEQ ID NOs:63, 138, 139) or a polynucleotide encoding said protein;
(ii) optionally determining whether the amount of protein differs from the amount of the corresponding protein quantified in a healthy subject; and
(iii) optionally correlating a change in expression of said protein when compared with the expression of said protein in a healthy subject with a neurological disease which is preferably selected from the group consisting of Alzheimer' s disease, multiple sclerosis or brain damage and Parkinsons' disease.

20. A polynucleotide encoding a polypeptide of claim 1.


SEQUENCE LISTING (160 sequences)

SEQ ID NO: 1 - protein, 645 aa, Homo sapiens
MSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 2 - protein, 364 aa, Mus musculus
MPHPYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHVYLEGTLLKPNMVTPGHACTQKFSNEEIAMATVTALRRTVPPAVTGVTFLSGGQSEEEASINLNAINKCPLLKPWALTFSYGRALQASALKAWGGKKENLKAAQEEYIKRALANSLACQGKYTPSGQSGAAASESLFISNHAY

SEQ ID NO: 3 - protein, 363 aa, Mus musculus
MPHSYPALSAEQKKELSDIALRIVTPGKGILAADESVGSMAKRLSQIGVENTEENRRLYRQVLFSADDRVKKCIGGVIFFHETLYQKDDNGVPFVRTIQDKGILVGIKVDKGVVPLAGTDGETTTQGLDGLLERCAQYKKDGADFAKWRCVLKISDRTPSALAILENANVLARYASICQQNGIVPIVEPEILPDGDHDLKRCQYVTEKVLAAVYKALSDHHVYLEGTLLKPNMVTPGHACPIKYSPEEIAMATVTALRRTVPPAVPGVTFLSGGQSEEEASLNLNAINRCPLPRPWALTFSYGRALQASALNAWRGQRDNAGAATEEFIKRAEMNGLAAQGRYEGSGDGGAAAQSLYIANHAY

SEQ ID NO: 4 - protein, 249 aa, Mus musculus
MAPTRKFFVGGNWKMNGRKKCLGELICTLNAANVPAGTEVVCAPPTAYIDFARQKLDPKIAVAAQNCYKVTNGAFTGEISPGMIKDLGATWVVLGHSERRHVFGESDELIGQKVSHALAEGLGVIACIGEKLDEREAGITEKVVFEQTKVIADNVKDWSKVVLAYEPVWAIGTGKTATPQQAQEVHEKLRGWLKSNVNDGVAQSTRIIYGGSVTGATCKELASQPDVDGFLVGGASLKPEFVDIINAKQ

SEQ ID NO: 5 - protein, 333 aa, Mus musculus
MVKVGVNGFGRIGHLVTRAAICSGKVEIVAINDPFIDLNYMVYMFQYDSTHGKFNGTVKAENGKLVINGKPITIFQERDPTNIKWGEAGAEYVVESTGVFTIMEKAGAHLKGGAKRVIISAPSADAPMFVMGVNHEKYDNSLKIVNNASCTTNCLAPLSKVIHDNFGIVEGLMTTVHAITATQKTVGGPSGKLWRDGRGAAQNIIPASTGAAKAVGKVIPELNGKLTGMAFRVPTPNVSVVDLTCRLEKPAKYDDIKKVVKQASEGPLKGILGYTEDQVVSCDFNSNSHSSTFDAGAVIALNDNFVKLISWYDNEYGYSNRVVDLMAYMASKE

SEQ ID NO: 6 - protein, 366 aa, Mus musculus
MLSLSPHFLSLQKVNVVEQEKIDKLMIEMDGTENKSKFGANAILGVSLAVCKAGAVEKGVPLYRHIADLAGNPEVILPVPAFNVINGGSHAGNKLAMQEFMILPVGASSFREAMRIGAEVYHNLKNVIKEKYGKDATNVGDEGGFAPNILENKEALELLKTAIAKAGYTDQVVIGMDVAASEFYRSGKYDLDFKSPDDPSRYITPDQLADLYKSFVQNYPVVSIEDPFDQDDWGAWQKFTASAGIQVVGDDLTVTNPKRIAKAASEKSCNCLLLKVNQIGSVTESLQACKLAQSNGWGVMVSHRSGETEDTFIADLVVGLCTGQIKTGAPCRSERLAKYNQILRIEEELGSKAKFAGRSFRNPLAK

SEQ ID NO: 7 - protein, 434 aa, Mus musculus
MSIEKIWAREILDSRGNPTVEVDLYTAKGLFRAAVPSGASTGIYEALELRDGDKQRYLGKGVLKAVDHINSRIAPALISSGISVVEQEKLDNLMLELDGTENKSKFGANAILGVSLAVCKAGAAERDLPLYRHIAQLAGNSDLILPVPAFNVINGGSHAGNKLAMQEFMILPVGAESFRDAMRLGAEVYHTLKGVIKDKYGKDATNVGDEGGFAPNILENSEALELVKEAIDKAGYTEKMVIGMDVAASEFYRDGKYDLDFKSPADPSRYITGDQLGALYQDFVRNYPVVSIEDPFDQDDWAAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGDEARFAGHNFRNPSVL

SEQ ID NO: 8 - protein, 334 aa, Mus musculus
MATLKEKLIASVADDEAAVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCTIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVDSAYEVIKLKGYTNWAIGLSVADLIESMLKNLSRIHPVSTMVKGMYGIENEVFLSLPCILNARGLTSVINQKLKDDEVAQLRKSADTLWDIQKDLKDL

SEQ ID NO: 9 - protein, 745 aa, Mus musculus
MAFQKAVKGTILVGGGALATVLGLSPFAHYRRKQVSLAYVEAAGYLTEPVNREPPSREAQLMTLKNTPEFDILVIGGGATGCGCALDAVTRGLKTALVERDDFSSGTSSRSTKLIHGGVRYLQKAIMNLDVEQYRMVKEALHERANLLEIAPHLSAPLPIMLPLYKWWQLPYYWVGIKMYDLVAGSQCLKSSYVLSKSRALEHFPMLQKDKLVGAIVYYDGQHNDARMNLAIALTAARYGAATANYMEVVSLLKKTDPETGKERVSGARCKDVLTGQEFDVRAKCVINASGPFTDSVRKMDDKNVVPICQPSAGVHIVMPGYYSPENMGLLDPATSDGRVIFFLPWEKMTIAGTTDTPTDVTHHPIPSEEDINFILNEVRNYLSSDVEVRRGDVLAAWSGIRPLVTDPKSADTQSISRNHVVDISDSGLITIAGGKWTTYRSMAEDTVDAAVKFHNLNAGPSRTVGLFLQGGKDWSPTLYIRLVQDYGLESEVAQHLAKTYGDKAFEVAKMASVTGKRWPVVGVRLVSEFPYIEAEVKYGIKEYACTAVDMISRRTRLAFLNVQAAEEALPRIVELMGRELNWSELRKQCVTYDSDGSTVATSWSSQEELETATRFLYYEMGYKSRTEQLTDSTEISLLPSDIDRYKKRFHKFDEDEKGFITIVDVQRVLESINVQMDENTLHEILCEVDLNKNGQVELHEFLQLMSAVQKGRVSGSRLAILMKTAEENLDRRVPIPVDRSCGGL

SEQ ID NO: 10 - protein, 373 aa, Mus musculus
MATSASSHLNKGIKQMYMSLPQGEKVQAMYIWVDGTGEGLRCKTRTLDCEPKCVEELPEWNFDGSSTFQSEGSNSDMYLHPVAMFRDPFRKDPNKLVLCEVFKYNRKPAESNLRHICKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWIARFILHRVCEDFGVIATFDPKPIPGNWNGAGCHTNFSTKAMREENGLKWIEEAIDKLSKRHQYHIRAYDPKGGLDNARRLTGFHETSNINDFSAGVANRGASIRIPRTVGQEKKGYFEDRRPSANCDPYAVTEAIVRTCLLNETGDEPFQYKN

SEQ ID NO: 11 - protein, 642 aa, Mus musculus
MWRVCARRARSAVPRDGFRARWAALKEGPGAPCGSPRIGPAAVRCGSGIPRYGVRSLCGWSSGSGTVPRNRLLRQLLGSPSRRSYSLPPHQKVPLPSLSPTMQAGTIARWEKKEGEKISEGDLIAEVETDKATVGFESLEECYMAKILVPEGTRDVPVGSIICITVEKPQDIEAFKNYTLDLAAAAAPQAAPAAAPATAAAPAAPSASAPGSSYPTHMQIVLPALSPTMTMGTVQRWEKKVGEKLSEGDLLAEIETDKATIGFEVQEEGYLAKILVPEGTRDVPLGAPLCIIVEKQEDIAAFADYRPTEVTSLKPQAAPPAPPPVAAVPPTPQPVAPTPSAAPAGPKGRVFVSPLAKKLAAEKGIDLTQVKGTGPEGRIIKKDIDSFVPSKAAPAAAAAMAPPGPRVAPAPAGVFTDIPISNIRRVIAQRLMQSKQTIPHYYLSVDVNMGEVLLVRKELNKMLEGKGKISVNDFIIKASALACLKVPEANSSWMDTVIRQNHVVDVSVAVSTPAGLITPIVFNAHIKGLETIASDVVSLASKAREGKLQPHEFQGGTFTISNLGMFGIKNFSAIINPPQACILAIGASEDKLIPADNEKGFDVASVMSVTLSCDHRVVDGAVGAQWLAEFKKYLEKPITMLL

SEQ ID NO: 12 - protein, 316 aa, Mus musculus
MKIFDAAKAPIQWEERNVTAIQGPGGKWMIPPEAKESMDKNKMGLKGPLKTPIAAGHPSMNLLLRKTFDLYANVRPCVSIEGYKTPYTDVNIVTIRENTEGEYSGIEHVIVDGVVQSIKLITEEASKRIAEFAFEYARNNHRSNVTAVHKANIMRMSDGLFLQKCREVAENCKDIKFNEMYLDTVCLNMVQDPSQFDVLVMPNLYGDILSDLCAGLIGGLGVTPSGNIGANGVAIFESVHGTAPDIAGKDMANPTALLLSAVMMLRHMGLFDHAAKIEAACFATIKDGKSLTKDLGGNAKCSDFTEEICRRVKDLD

SEQ ID NO: 13 - protein, 334 aa, Mus musculus
MSEPIRVLVTGAAGQIAYSLLYSIGNGSVFGKDQPIILVLLDITPMMGVLDGVLMELQDCALPLLQDVIATDKEEIAFKDLDVAVLVGSMPRREGMERKDLLKANVKIFKSQGTALEKYAKKSVKVIVVGNPANTNCLTASKSAPSIPKENFSCLTRLDHNRAKSQIALKLGVTADDVKNVIIWGNHSSTQYPDVNHAKVKLQGKEVGVYEALKDDSWLKGEFITTVQQRGAAVIKARKLSSAMSAAKAIADHIRDIWFGTPEGEFVSMGVISDGNSYGVPDDLLYSFPVVIKNKTWKFVEGLPINDFSREKMDLTAKELTEEKETAFEFLSSA

SEQ ID NO: 14 - protein, 172 aa, Mus musculus
MPGIVELPTLEELKVEEVKVSSAVLKAAAHHYGAQCDKTNKEFMLCRWEEKDPRRCLKEGKLVNGCALNFFRQIKSHCAEPFTEYWTCLDYSNMQLFRHCRQQQAKFDQCVLDKLGWVRPDLGQLSKVTKVKTDRPLPENPYHSRARPEPNPVIEGDLKPAKHGTRFFFWTV

SEQ ID NO: 15 - protein, 727 aa, Mus musculus
MLRIPIKRALIGLSNSPKGYVRTTGTAASNLIEVFVDGQSVMVEPGTTVLQACEKVGMQIPRFCYHERLSVAGNCRMCLVEIEKAPKVVAACAMPVMKGWNILTNSEKSKKAREGVMEFLLANHPLDCPICDQGGECDLQDQSMMFGSDRSRFLEGKRAVEDKNIGPLVKTIMTRCIQCTRCIRFASEIAGVDDLGTTGRGNDMQVGTYIEKMFMSELSGNVIDICPVGALTSKPYAFTARPWETRKTESIDVMDAVGSNIVVSTRTGEVMRILPRMHEDINEEWISDKTRFAYDGLKRQRLTEPMVRNEKGLLTYTSWEDALSRVAGMLQNFEGNAVAAIAGGLVDAEALVALKDLLNKVDSDNLCTEEIFPTEGAGTDLRSNYLLNTTIAGVEEADVVLLVGTNPRFEAPLFNARIRKSWLHNDLKVALIGSPVDLTYRYDHLGDSPKILQDIASGRHSFCEVLKDAKKPMVVLGSSALQRDDGAAILAAVSNMVQKIRVTTGVAAEWKVMNILHRIASQVAALDLGYKPGVEAIRKNPPKMLFLLGADGGCITRQDLPKDCFIVYQGHHGDVGAPMADVILPGAAYTEKSATYVNTEGRAQQTKVAVTPPGLAREDWKIIRALSEIAGITLPYDTLDQVRNRLEEVSPNLVRYDDIEETNYFQQASELAKLVNQEVLADPLVPPQLTIKDFYMTDSISRASQTMAKCVKAVTEGAQAVEEPSIC

SEQ ID NO: 16 - protein, 212 aa, Mus musculus
MYRLSSSMLPRALAQAMRTGHLNGQSLHSSAVAATYKYVNKKEQESEVDMKSATDNAARILMWTELIRGLGMTLSYLFREPATINYPFEKGPLSPRFRGEHALRRYPSGEERCIACKLCEAICPAQAITIEAEPRADGSRRTTRYDIDMTKCIYCGFCQEACPVDAIVEGPNFEFSTETHEELLYNKEKLLNNGDKWEAEIAANIQADYLYR

SEQ ID NO: 17 - protein, 480 aa, Mus musculus
MAASAVCRAACSGTQVLLRTRRSPALLRLPALRGTATFAQALQSVPETQVSILDNGLRVASEQSSHATCTVGVWIDAGSRYETEKNNGAGYFLEHLAFKGTKNRPGNALEKEVESIGAHLNAYSTREHTAYLIKALSKDLPKVVELLADIVQNSSLEDSQIEKERDVILREMQENDASMQNVVFDYLHATAFQGTPLAQAVEGPSENVRRLSRTDLTDYLNRNYKAPRMVLAAAGGVEHQQLLDLAQKHLSSVSRVYEEDAVPGLTPCRFTGSEIRHRDDALPLAHVAIAVEGPGWANPDNVTLQVANAIIGHYDCTCGGGVHLSSPLASVAVANKLCQSFQTFNISYSDTGLLGAHFVCDAMSIDDMVFFLQGQWMRLCTSATESEVTRGKNILRNALVSHLDGTTPVCEDIGRSLLTYGRRIPLAEWESRIQEVDAQMLRDICSKYFYDQCPAVAGYGPIEQLPDYNRIRSGMFWLRF

SEQ ID NO: 18 - protein, 161 aa, Mus musculus
MAGRKLALKTIDWVSFVEVMPQNQKAIGNALKSWNETFHARLASLSEKPPAIDWAYYRANVAKPGLVDDFEKKYNALKIPVPEDKYTALVDQEEKEDVKSCAEFVSGSQLRIQEYEKQLEKMRNIIPFDQMTIDDLNEIFPETKLDKKKYPYWPHQPIENL

SEQ ID NO: 19 - protein, 381 aa, Mus musculus
MPFSNSHNTQKLRFPAEDEFPDLSSHNNHMAKVLTPELYAELRAKCTPSGFTLDDAIQTGVDNPGHPYIMTVGAVAGDEESYDVFKDLFDPIIEERHGGYQPSDEHKTDLNPDNLQGGDDLDPNYVLSSRVRTGRSIRGFCLPPHCSRGERRAIEKLAVEALSSLDGDLSGRYYALKSMTEAEQQQLIDDHFLFDKPVSPLLLASGMARDWPDARGIWHNDNKTFLVWINEEDHLRVISMQKGGNMKEVFTRFCTGLTQIETLFKSKNYEFMWNPHLGYILTCPSNLGTGLRAGVHIKLPHLGKHEKFSEVLKRLRLQKRGTGGVDTAAVGGVFDVSNADRLGFSEVELVQMVVDGVKLLIEMEQRLEQGQAIDDLMPAQK

SEQ ID NO: 20 - protein, 646 aa, Mus musculus
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIISWLDKNQTAEEEEFEHQQKELEKVCNPIITKLYQSAGGMPGGMPGGFPGGGAPPSGGASSGPTIEEVD

SEQ ID NO: 21 - protein, 679 aa, Mus musculus
MISASRAAAARLVGTAASRSPAAARPQDGWNGLSHEAFRFVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDTKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDASGPKHLNMKLTRAQFEGIVTDLIKRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRALLAGKDSETGENIRQAASSLQQASLKLFEMAYKKMASEREGSGSSGTGEQKEDQKEEKQ

SEQ ID NO: 22 - protein, 679 aa, Mus musculus
MISASRAAAARLVGTAASRSPAAARPQDGWNGLSHEAFRFVSRRDYASEAIKGAVVGIDLGTTNSCVAVMEGKQAKVLENAEGARTTPSVVAFTADGERLVGMPAKRQAVTNPNNTFYATKRIIGRRYDDPEVQKDTKNVPFKIVRASNGDAWVEAHGKLYSPSQIGAFVLMKMKETAENYLGHTAKNAVITVPAYFNDSQRDATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDASGPKHLNMKLTRAQFEGIVTDLIKRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRALLAGKDSETGENIRQAASSLQQASLKLFEMAYKKMASEREGSGSSGTGEQKEDQKEEKQ

SEQ ID NO: 23 - protein, 505 aa, Mus musculus
MRFSCLALLPGVALLLASARLAAASDVLELTDENFESRVSDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASVVGFFRDLFSDGHSEFLKAASNLRDNYRFAHTNIESLVKEYDDNGEGITIFRPLHLANKFEDKTVAYTEKKMTSGKIKKFIQDSIFGLCPHMTEDNKDLIQGKDLLTAYYDVDYEKNAKGSNYWRNRVMMVAKKFLDAGHKLNFAVASRKTFSHELSDFGLESTTGEVPVVAIRTAKGEKFVMQEEFSRDGKALEQFLQEYFDGNLKRYLKSEPIPESNEGPVKVVVAENFDDIVNEEDKDVLIEFYAPWCGHCKNLEPKYKELGEKLSKDPNIVIAKMDATANDVPSPYEVKGFPTIYFSPANKKLTPKKYEGGRELNDFISYLQREATNPPIIQEEKPKKKKKAQEDL

SEQ ID NO: 24 - protein, 617 aa, Mus musculus
MDFSKLPKIRDEDKESTFGYVHGVSGPVVTACDMAGAAMYELVRVGHSELVGEIIRLEGDMATIQVYEETSGVSVGDPVLRTGKPRSVELGPGIMGAIFDGIQRPLSDISSQTQSIYIPRGVNVSALSRDIKWEFIPSKNLRVGSHITGGDIYGIVNENSLIKHKIMLPPRNRGSVTYIAPPGNYDASNVVLELEFEGVKEKFSMVQVWPVRQVRPVTEKLPANHPLLTGQRVLDALFPCVQGGTTAIPGAFGCGKTVISQSLSKYSNSDVIIYVGCGERGNEMSEVLRDFPELTMEVDGKAESIMKRTALVANTSNMPVAAREASIYTGITLSEYFRDMGYHVSMMADSTSRWAEALREISGRLAEMPADSGYPAYLGARLASFYERAGRVKCLGNPEREGSVSIVGAVSPPGGDFSDPVTSATLGIVQVFWGLDKKLAQRKHFPSVNWLISYSKYMRALDEYYDKHFTEFVPLRTKAKEILQEEGDLAEIVQLVGKASLAETDKITLEVAKLIKDDFLQQNGYTPYDRFCPFYKTVGMLSNMISFYDMARRAVETTAQSDNKITWSIIREHMGEILYKLSSMKFKDPVKDGEAKIKADYAQLLEDMQNAFRSLED

SEQ ID NO: 25 - protein, 418 aa, Mus musculus
MEEIGILVEKIQDEIPALSVSRPQTGLSFLGPEPEDLEDLYSRYKKLQQELEFLEVQEEYIKDEQKNLKKEFLHAQEEVKRIQSIPLVIGQFLEAVDQNTAIVGSTTGSNYYVRILSTIDRELLKPNASVALHKHSNALVDVLPPEADSSIMMLTSDQKPDVMHADIGGMDIQKQEVREAVELPLTHFELYKQIGIDPPRGVLMYGPPGCGKTMLAKAVAHHTTAAFIRVVGSEFVQKYLGEGPRMVRDVFRLAKENAPAIIFIDEIDAIATKRFDAQTGADREVQRILLELLNQMDGFDQNVNVKVIMATNRADTLDPALLRPGRLDRKIEFPLPDRRQKRLIFSTITSKMNLSEEVDLEDYVARPDKISGADINSICQESGMLAVRENRYIVLAKDFEKAYKTVIKKDEQEHEFYK

SEQ ID NO: 26 - protein, 234 aa, Mus musculus
MAKRGYSFSLTTFSPSGKLVQIEYALAAVAGGAPSVGIKAANGVVLATEKKQKSILYDERSVHKVEPITKHIGLVYSGMGPDYRVLVHRARKLAQQYYLVYQEPIPTAQLVQRVASVMQEYTQSGGVRPFGVSLLICGWNEGRPYLFQSDPSGAYFAWKATAMGKNYVNGKTFLEKRYNEDLELEDAIHTAILTLKESFEGQMTEDNIEVGICNEAGFRRLTPTEVRDYLAAIA

SEQ ID NO: 27 - protein, 233 aa, Mus musculus
GLCLQVPSAKMQLKPMEINPEMLNKVLAKLGVAGQWRFADVLGLEEETLGSVPSPACALLLLFPLTAQHENFRKKQIEELKGQEVSPKVYFMKQTIGNSCGTIGLIHAVANNQDKLEFEDGSVLKQFLSETEKLSPEDRAKCFEKNEAIQAAHDSVAQEGQCRVDDKVNFHFILFNNVDGHLYELDGRMPFPVNHGASSEDSLLQDAAKVCREFTEREQGEVRFSAVALCKAA

SEQ ID NO: 28 - protein, 822 aa, Mus musculus
PLPSGRHSRRPGEARAMASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYG

SEQ ID NO: 29 - protein, 334 aa, Mus musculus
MAASLGFRGAASGLWYWSGRRRPVGSLAAVCSRSMASKTPVGFIGLGNMGNPMAKNLMKHGYPLILYDVFPDVCKEFKEAGEQVASSPAEVAEKADRIITMLPSSMNAVEVYSGANGILKKVKKGSLLIDSSTMILQFQKNGKRSEKMGAVFMDAPVSGGVGAARSGNLTFMVGGVEDEFAAAQELLECMGSNVVYCGAVGTGQSAQICNNMLLAISMIGTAEAMNLGIRSGLDPKLLAKILNMSSGRCWSSDTYNPVPGFMHGVPSSNNYQGGFGTTLMAKDLGLAQDSATSTKTPILLGSLAHQIYRMMCSKGYSKKDFSSVFQYLREEEPF

SEQ ID NO: 30 - protein, 291 aa, Mus musculus
MATATVRPAAQRLRLLLSPLKSRICVPQAEPVATFGTAVTSAKVAVNGVHLHYQRVGEGEHAILLLPGMLGSGKTDFAPQLQSLNKKRFTLVAWDPRGYGYSRPPDRDFPRDFFERDAKDAVDLMKALQFKQVSLLGWSDGGITALIAAAKYPSYIRKMVIWGANAYVTEEDSRIYQGIRDVSKWSEKARKPLEALYGYDYLAKTCEDWVDGISQFKQLPEGNICRHLLPLVQCPTLIVHGEKDPLVPRFHADFLLQHVKGSRLHLMPEGKHNLHLRFADEFNRLVEDFLQ

SEQ ID NO: 31 - protein, 259 aa, Mus musculus
MAARRALKAVLVDLNGTLHIEDAAVPGAQEALKRLRATSVMVRFVTNTTKETKKDLLERLKKLEFEISEDEIFTSLTAARNLIEQKQVRPMLLVDDRALPEFTGVQTQDPNAVVIGLAPEHFHYQLLNQAFRLLLDGAPLIAIHKARYYKRKDGLALGPGPFVTALEYATDTKAMVVGKPEKTFFLEALRDADCAPEEAVMIGDDCRDDVDGAQNIGMLGILVKTGKYKAADEEKINPPPYLTCESFPHAVDHILQHLL

SEQ ID NO: 32 - protein, 349 aa, Mus musculus
PRAVFPSIVGRSRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 33 - protein, 368 aa, Mus musculus
LVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 34 - protein, 146 aa, Mus musculus
MAGWQSYVDNLMCDGCCQEAAIVGYCDAKYVWAATAGGVFQSITPVEIDMIVGKDREGFFTNGLTLGAKKCSVIRDSLYVDGDCTMDIRTKSQGGEPTYNVAVGRAGRALVIVMGKEGVHAGTINKKTYELALYLKRSVTNLYLAS

SEQ ID NO: 35 - protein, 199 aa, Mus musculus
MANRGPSYGLSREVQEKIEQKYDADLENKLVDWIILQCAEDIEHPPPGRAHFQKWLMDGTVLCKLINSLYPPGQEPIPKISESKMAFKQMEQISQFLKAAEVYGVRTTDIFQTVDLWEGKDMAAVQRTLMALGSVAVTKDDGCYRGEPSWFHRKAQQNRRGFSEEQLRQGQNVIGLQMGSNKGASQAGMTGYGMPRQIM

SEQ ID NO: 36 - protein, 677 aa, Mus musculus
RVVSAAEPETMAKIAQGAMYRGSVHDFPEFDANQDAEALYTAMKGFGSDKESILELITSRSNKQRQEICQNYKSLYGKDLIEDLKYELTGKFERLIVNLMRPLAYCDAKEIKDAISGIGTDEKCLIEILASRTNEQMHQLVAAYKDAYERDLESDIIGDTSGHFQKMLVVLLQGTRENDDVVSEDLVQQDVQDLYEAGELKWGTDEAQFIYILGNRSKQHLRLVFDEYLKTTGKPIEASIRGELSGDFEKLMLAVVKCIRSTPEYFAERLFKAMKGLGTRDNTLIRIMVSRSELDMLDIREIFRTKYEKSLYSMIKNDTSGEYKKALLKLCGGDDDAAGQFFPEAAQVAYQMWELSAVSRVELKGTVCAANDFNPDADAKALRKAMKGIGTDEATIIDIVTHRSNAQRQQIRQTFKSHFGRDLMADLKSEISGDLARLILGLMMPPAHYDAKQLKKAMEGAGTDEKTLIEILATRTNAEIRAINEAYKEDYHKSLEDALSSDTSGHFRRILISLATGNREEGGENRDQAQEDAQEIADTPSGDKTSLETRFMTVLCTRSYPHLRRVFQEFIKKTNYDIEHVIKKEMSGDVKDAFVAIVQSVKNKPLFFADKLYKSMKGAGTDEKTLTRVMVSRSEIDLLNIRREFIEKYDKSLHQAIEGDTSGDFMKALLALCGGED

SEQ ID NO: 37 - protein, 501 aa, Mus musculus
MSFGSEHYLCSASSYRKVFGDSSRLSARLSGPGGSGSFRSQSLSRSNVASTAACSSASSLGLGLAYRRLPASDGLDLSQAAARTNEYKIIRTNEKEQLQGLNDRFAVFIEKVHQLETQNRALEAELAALRQRHAEPSRVGELFQRELRELRAQLEEASSARAQALLERDGLAEEVQRLRARCEEESRGREGAERALKAQQRDVDGATLARLDLEKKVESLLDELAFVRQVHDEEVAELLATLQASSQAAAEVDVAVAKPDLTSALREIRAQYESLAAKNLQSAEEWYKSKFANLNEQAARSTEAIRASREEIHEYRRQLQARTIEIEGLRGANESLERQILELEERHSAEVAGYQDSIGQLESDLRNTKSEMARHLREYQDLLNVKMALDIEIAAYRKLLEGEETRFSTGGLSISGLNPLPNPSYLLPPRILSSTASKVSSAGLSLKKEEEEEEEEASKEVSKKTSKVGEGFEETLGEAVISTKKTGKSATEESTSSSQKM

SEQ ID NO: 38 - protein, 543 aa, Mus musculus
MSSFGYDPYFSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEIAFLKKVHEEEIAELQAQIQYAQISVEMDVSSKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRSTKSEMARYLKEYQDLLNVKMALDIEIAAYRKLLEGEETRLSFTSVGSITSGYSQSSQVFGRSAYSGLQSSSYLMSARSFPAYYTSHVQEEQTEVEETIEATKAEEAKDEPPSEGEAEEEEKEKEEGEEEEGAEEEEAAKDESEDTKEEEEGGEGEEEDTKESEEEEKKEESAGEEQVAKKKD

SEQ ID NO: 39 - protein, 400 aa, Mus musculus
MPRFSLSRMTPPLPARVDFSLAGALNAGFKETRASERAEMMELNDRFASYIEKVRFLEQQNKALAAELNQLRAKEPTKLADVYQAELRELRLRLDQLTANSARLEVERDNFAQDLGTLRQKLQDETNLRLEAENNLAAYRQEADEATLARVDLERKVESLEEEIQFLRKIYEEEVRELREQLAQQQVHVEMDVAKPDLTAALREIRTQYEAVATSNMQETEEWYRSKFADLTDAASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKDSKQEHKDVVM

SEQ ID NO: 40 - protein, 451 aa, Mus musculus [sequence omitted]

SEQ ID NO: 41 - protein, 445 aa, Mus musculus [sequence omitted]

SEQ ID NO: 42 - protein, 450 aa, Mus musculus
MREIVHIQAGQCGNQIGAKFWEVISDEHGIDPSGNYVGDSDLQLERISVYYNEASSHKYVPRAILVDLEPGTMDSVRSGAFGHLFRPDNFIFGQSGAGNNWAKGHYTEGAELVDSVLDVVRKECENCDCLQGFQLTHSLGGGTGSGMGTLLISKVREEYPDRIMNTFSVVPSPKVSDTVVEPYNATLSIHQLVENTDETYCIDNEALYDICFRTLKLATPTYGDLNHLVSATMSGVTTSLRFPGQLNADLRKLAVNMVPFPRLHFFMPGFAPLTARGSQQYRALTVPELTQQMFDAKNMMAACDPRHGRYLTVATVFRGRMSMKEVDEQMLAIQSKNSSYFVEWIPNNVKVAVCDIPPRGLKMSSTFIGNSTAIQELFKRISEQFTAMFRRKAFLHWYTGEGMDEMEFTEAESNMNDLVSEYQQYQDATAEEEGEMYEDDDEESEAQGPK

SEQ ID NO: 43 - protein, 572 aa, Mus musculus
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDITEWHKGIQEEMEALVKDHGVNSFLVYMAFKDRFQLTDSQIYEVLSVIRDIGAIAQVHAENGDIIAEEQQRILDLGITGPEGHVLSRPEEVEAEAVNRSITIANQTNCPLYVTKVMSKSAAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRISVGSDADLVIWDPDSVKTISAKTHNSALEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 44 - protein, 365 aa, Mus musculus
EQKRLLEQGITGPEGHVLSHPEEVEAEAVYRAVTIAKQANCPLYVTKVMSKGAADMVAQAKRRGVVVFGEPITASLGTDGSHYWSKNWAKAAAFVTSPPINPDPTTADHLTSLLSSGDLQVTGSAHCTFTTAQKAVGKDNFTLIPEGVNGIEERMSVVWEKCVASGKMDENEFVAVTSTNAAKIFNFYPRKGRVAVGSDADLVIWNPRATKVISAKSHNLNVEYNIFEGVECRGVPTVVISQGRVVLEDGNLLVTPGAGRFIPRKTFPDFVYKRIKARNRLAEIHGVPRGLYDGPVHEVMLPAKPGSGTQARASCSGKISVPPVRNLHQSGFSLSGSQADDHIARRTAQKIMAPPGGRSNITSLS

SEQ ID NO: 45 - protein, 226 aa, Mus musculus
MGGKLSKKKKGYNVNDEKAKDKDKKAEGAGTEEEGTPKESEPQAAADATEVKESTEEKPKDAADGEAKAEEKEADKAAAAKEEAPKAEPEKSEGAAEEQPEPAPAPEQEAAAPGPAAGGEAPKAGEASAESTGAADGAAPEEGEAKKTEAPAAAGPEAKSDAAPAASDSKPSSAEPAPSSKETPAASEAPSSAAKAPAPAAPAAAEPQAEAPAAAASSEQSVAVKE

SEQ ID NO: 46 - protein, 189 aa, Mus musculus
MNPEYDYLFKLLLIGDSGVGKSCLLLRFADDTYTESYISTIGVDFKIRTIELDGKTIKLQIWDTAGQERFRTITSSYYRGAHGIIVVYDVTDQESYANVKQWLQEIDRYASENVNKLLVGNKSDLTTKKVVDNTTAKVSRPALAVRGLGPTCLHPFSPCSPLSLLPFAGICRLSGCPLPRDKCQECHQC

SEQ ID NO: 47 - protein, 220 aa, Mus musculus
MASATDSRYGQKESSDQNFDYMFKILIIGNSSVGKTSFLFRYADDSFTPAFVSTVGIDFKVKTIYRNDKRIKLQIWDTAGQERYRTITTAYYRGAMGFILMYDITNEESFNAVQDWSTQIKTYSWDNAQVLLVGNKCDMEDERVVSSERGRQLADHLGFEFFEASAKDNINVKQTFERLVDVICEKMSESLDTADPAVTGAKQGPQLTDQQAPPHQDCAC

SEQ ID NO: 48 - protein, 208 aa, Mus musculus
MSAGGDFGNPLRKFKLVFLGEQSVGKTSLITRFMYDSFDNTYQATIGIDFLSKTMYLEDRTVRLQLWDTAGQERFRSLIPSYIRDSTVAVVVYDITNVNSFQQTTKWIDDVRTERGSDVIIMLVGNKTDLADKRQVSIEEGERKAKELNVMFIETSAKAGYNVKQLFRRVAAALPGMESTQDRSREDMIDIKLEKPQEQPVNEGGCSC

SEQ ID NO: 49 - protein, 447 aa, Mus musculus
MDEEYDVIVLGTGLTECILSGIMSVNGKKVLHMDRNPYYGGESSSITPLEELYKRFQILEGPPESMGRGRDWNVDLIPKFLMANGQLVKMLLYTEVTRYLDFKVVEGSFVYKGGKIYKVPSTETEALASNLMGMFEKRRFRKFLVFVANFDENDPKTFEGVDPQNTSMRDVYRKFDLGQDVIDFTGHALALYRTDDYLDQPCLETINRIKLYSESLARYGKSPYLYPLYGLGELPQGFARLSAIYGGTYMLNKPVDDIIMENGKVVGVKSEGEVARCKQLICDPSYIPDRVQKAGQVIRIICILSHPIKNTNDANSCQIIIPQNQVNRKSDIYVCMISYAHNVAAQGKYIAIASTTVETAEPEKEVEPALELLEPIDQKFVAISDLYEPIDDGSESQVFCSCSYDATTHFETTCNDIKDIYKRMAGSAFDFENMKRKQNDVFGEADQ

SEQ ID NO: 50 - protein, 298 aa, Mus musculus
MDNAGKEREAVQLMAEAEKRVKASHSFLRGLFGGNTRIEEACEMYTRAANMFKMAKNWSAAGNAFCQAAKLHMQLQSKHDSATSFVDAGNAYKKADPQEAINCLNAAIDIYTDMGRFTIAAKHHITIAEIYETELVDIEKAIAHYEQSADYYKGEESNSSANKCLLKVAAYAAQLEQYQKAIEIYEQVGANTMDNPLLKYSAKDYFFKAALCHFIVDELNAKLALEKYEEMFPAFTDSRECKLLKKLLEAHEEQNSEAYTEAVKEFDSISRLDQWLTTMLLRIKKSIQGDGEGDGDLK

SEQ ID NO: 51 - protein, 504 aa, Mus musculus
MRFSCLALLPGVALLLASARLAAASDVLELTDENFESRVSDTGSAGLMLVEFFAPWCGHCKRLAPEYEAAATRLKIVPLAKVDCTANTNTCNKYGVSGYPTLKIFRAGEEAGAYDGPRTADGIVSHLKKQAGPASVPLRTEEEFKKFISDKDASVVGFFRDLFSDGHSEFLKAASNLRDNYRFAHTNIESLVKEYDDNGEGITIFRPLHLANKFEDKTVAYTEKKMTSAKIKKFIQDSIFGLCPHMTEDNKDLIQGKDLLTAYYDVDYEKNAKGSNYWRNRVMMVAKKFLDAGHKLNFAVASRKTFSHELSDFSLESTTGEVPVVAIRTAKGEKFVMQEEFSRDGKALEQFLQEYFDGNLKRYLKSEPIPESNEGPVKVVVAENFDDIVNEEDKDVLIEFYAPWCGHCKNLEPKYKELGEKLSKDPNIVIAKMDATANDVPSPYEVKGFPTIYFSPANKKLTPKKYEGGRELNDFISYLQREATNPPIIQEEKPKKKKKAQEDL

SEQ ID NO: 52 - protein, 157 aa, Rattus norvegicus
MVDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDV

SEQ ID NO: 53 - protein, 261 aa, Mus musculus
MAESHLQSSLITASQFFEIWLHFDADGSGYLEGKELQNLIQELLQARKKAGLELSPEMKSFVDQYGQRDDGKIGIVELAHVLPTEENFLLLFRCQQLKSCEEFMKTWRKYDTDHSGFIETEELKNFLKDLLEKANKTVDDTKLAEYTDLMLKLFDSNNDGKLELTEMARLLPVQENFLLKFQGIKMCGKEFNKAFELYDQDGNGYIDENELDALLKDLCEKNKQELDINNITTYKKNIMALSDGGKLYRTDLALILSAGDN

SEQ ID NO: 54 - protein, 190 aa, Mus musculus
KIEMAELAQILPTEENFLLCFRQHVGSSAEFMEAWRKYDTDRSGYIEANELKGFLSDLLKKANRPYDEPKLQEYTQTILRMFDLNGDGKLGLSEMSRLLPVQENFLLKFQGMKLTSEEFNAIFTFYDKDGSGYIDENELDALLKDLYEKNKKEMNIQQLTTYRKSVMSLAEAGKLYRKDLEIVLCSEPPV

SEQ ID NO: 55 - protein, 191 aa, Mus musculus
MGKQNSKLAPEVMEDLVKSTEFNEHELKQWYKGFLKDCPSGRLNLEEFQQLYVKFFPYGDASKFAQHAFRTFDKNGDGTIDFREFICALSITSRGSFEQKLNWAFNMYDLDGDGKITRVEMLEIIEAIYKMVGTVIMMKMNEDGLTPEQRVDKIFSKMDKNKDDQITLDEFKEAAKSDPSIVLLLQCDIQK

SEQ ID NO: 56 - protein, 253 aa, Mus musculus
MALSMPLNGLKEEDKEPLIELFVKAGSDGESIGNCPFSQRLFMILWLKGVVFSVTTVDLKRKPADLQNLAPGTHPPFITFNSEVKTDVNKIEEFLEEVLCPPKYLKLSPKHPESNTAGMDIFAKFSAYIKNSRPEANEALERGLLKTLQKLDEYLNSPLPDEIDENSMEDIKFSTRRFLDGDEMTLADCNLLPKLHIVKVVAKKYRNFDIPKGMTGIWRYLTNAYSRDEFTNTCPSDKEVEIAYSDVAKRLTK

SEQ ID NO: 57 - protein, 187 aa, Mus musculus
MAADISQWAGPLCLQEVDEPPQHALRVDYAGVTVDELGKVLTPTQVMNRPSSISWDGLDPGKLYTLVLTDPDAPSRKDPKFREWHHFLVVNMKGNDISSGTVLSDYVGSGPPSGTGLHRYVWLVYEQEQPLSCDEPILSNKSGDNRGKFKVETFRKKYNLGAPAAGTCYQAKWDDYVPKLYKQLSGK

SEQ ID NO: 58 - protein, 256 aa, Mus musculus
GRFLQPEEYPVMDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQPESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 59 - protein, 175 aa, Mus musculus
MIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 60 - protein, 199 aa, Mus musculus
MSSGNAKIGYPAPNFKATAVMPDGQFKDISLSEYKGKYVVFFFYPLDFTFVCPTEIIAFSDRADEFKKLNCQVIGASVDSHFCHLAWINTPKKQGGLGPMNIPLISDPKRTIAQDYGVLKADEGISFRGLFIIDDKGILRQITINDLPVGRSVDEIIRLVQAFQFTDKHGEVCPAGWKPGSDTIKPDVNKSKEYFSKQK

SEQ ID NO: 61 - protein, 257 aa, Mus musculus
MAAAAGRLLWSSVARHASAISRSISASTVLRPVASRRTCLTDILWSASAQGKSAFSTSSSFHTPAVTQHAPYFKGTAVVNGEFKELSLDDFKGKYLVLFFYPLDFTFVCPTEIVAFSDKANEFHDVNCEVVAVSVDSHFSHLAWINTPRKNGGLGHMNITLLSDITKQISRDYGVLLESAGIALRGLFIIDPNGVVKHLSVNDLPVGRSVEETLRLVKAFQFVETHGEVCPANWTPESPTIKPSPTASKEYFEKVHQ

SEQ ID NO: 62 - protein, 312 aa, Mus musculus
MEGECRVLSIQSHVVRGYVGNRAAMFPLQVLGFEVDAVNSVQFSNHTGYAHWKGQVLKSQELHELYEGLKVNDVNKYDYVLTGYTRDKSFLAMVVDIVRELKQQNSRLVYVCDPVMGDKWNGEGSMYVPQDLLPVYRDKVVPVADIITPNQFEAELLSGRKIHSQEEAFEVMDMLHCMGPDTVVITSSDLPSSQGSDYLIALGSQRMRKPDGSTVTQRIRMEMRKVEAVFVGTGDLFAAMLLAWTHKHPDNLKVACEKTVSAMQHVLQRTIRCAKAEAGEGQKPSPAQLELRMVQSKRDIEDPEIVVQATVL

SEQ ID NO: 63 - protein, 354 aa, Mus musculus
MGCTLSAEERAALERSKAIEKNLKEDGISAAKDVKLLLLGAGESGKSTIVKQMKIIHEDGFSGEDVKQYKPVVYSNTIQSLAAIVRAMDTLGVEYGDKERKTDSKMVCDVVSRMEDTEPFSAELLSAMMRLWGDSGIQECFNRSREYQLNDSAKYYLDSLDRIGAGDYQPTEQDILRTRVKTTGIVETHFTFKNLHFRLFDVGGQRSERKKWIHCFEDVTAIIFCVALSGYDQVLHEDETTNRMHESLKLFDSICNNKWFTDTSIILFLNKKDIFEEKIKKSPLTICFPEYTGPSAFTEAVAHIQGQYESKNKSAHKEVYSHVTCATDTNNIQFVFDAVTDVIIAKNLRGCGLY

SEQ ID NO: 64 - DNA, 2199 bp, Homo sapiens
gggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaagcatcttgggattgaatttatggaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgaaaaaaaaaaaaaaaa

SEQ ID NO: 65 - protein, 299 aa, Mus musculus
MEGKAEQQGAGLTMAEGGEKEEFCFTAIYISGQWREPCVCTDLQRLEPGTMAPTRKFFVGGNWKMNGRKKCLGELICTLNAANVPAGTEVVCAPPTAYIDFARQKLDPKIAVAAQNCYKVTNGAFTGEISPGMIKDLGATWVVLGHSERRHVFGESDELIGQKVSHALAEGLGVIACIGEKLDEREAGITEKVVFEQTKVIADNVKDWSKVVLAYEPVWAIGTGKTATPQQAQEVHEKLRGWLKSNVNDGVAQSTRIIYGGSVTGATCKELASQPDVDGFLVGGASLKPEFVDIINAKQ

SEQ ID NO: 66 - protein, 642 aa, Mus musculus
MWRVCARRARSAVPRDGFRARWAALKEGPGAPCGSPRIGPAAVRCGSGIPRYGVRSLCGWSSGSGTVPRNRLLRQLLGSPSRRSYSLPPHQKVPLPSLSPTMQAGTIARWEKKEGEKISEGDLIAEVETDKATVGFESLEECYMAKILVPEGTRDVPVGSIICITVEKPQDIEAFKNYTLDLAAAAAPQAAPAAAPAPAAAPAAPSASAPGSSYPTHMQIVLPALSPTMTMGTVQRWEKKVGEKLSEGDLLAEIETDKATIGFEVQEEGYLAKILVPEGTRDVPLGAPLCIIVEKQEDIAAFADYRPTEVTSLKPQAAPPAPPPVAAVPPTPQPVAPTPSAAPAGPKGRVFVSPLAKKLAAEKGIDLTQVKGTGPEGRIIKKDIDSFVPSKAAPAAAAAMAPPGPRVAPAPAGVFTDIPISNIRRVIAQRLMQSKQTIPHYYLSVDVNMGEVLLVRKELNKMLEGKGKISVNDFIIKASALACLKVPEANSSWMDTVIRQNHVVDVSVAVSTPAGLITPIVFNAHIKGLETIASDVVSLASKAREGKLQPHEFQGGTFTISNLGMFGIKNFSAIINPPQACILAIGASEDKLIPADNEKGFDVASVMSVTLSCDHRVVDGAVGAQWLAEFKKYLEKPITMLL

SEQ ID NO: 67 - protein, 727 aa, Mus musculus
MLRIPIKRALIGLSNSPKGYVRTTGTAASNLIEVFVDGQSVMVEPGTTVLQACEKVGMQIPRFCYHERLSVAGNCRMCLVEIEKAPKVVAACAMPVMKGWNILTNSEKSKKAREGVMEFLLANHPLDCPICDQGGECDLQDQSMMFGSDRSRFLEGKRAVEDKNIGPLVKTIMTRCIQCTRCIRFASEIAGVDDLGTTGRGNDMQVGTYIEKMFMSELSGNVIDICPVGALTSKPYAFTARPWETRKTESIDVMDAVGSNIVVSTRTGEVMRILPRMHEDINEEWISDKTRFAYDGLKRQRLTEPMVRNEKGLLTYTSWEDALSRVAGMLQNFEGNAVAAIAGGLVDAEALVALKDLLNKVDSDNLCTEEIFPTEGAGTDLRSNYLLNTTIAGVEEADVVLLVGTNPRFEAPLFNARIRKSWLHNDLKVALIGSPVDLTYRYDHLGDSPKILQDIASGRHSFCEVLKDAKKPMVVLGSSALQRDDGAAILVAVSNMVQKIRVTTGVAAEWKVMNILHRIASQVAALDLGYKPGVEAIRKNPPKMLFLLGADGGCITRQDLPKDCFIVYQGHHGDVGAPMADVILPGAAYTEKSATYVNTEGRAQQTKVAVTPPGLAREDWKIIRALSEIAGITLPYDTLDQVRNRLEEVSPNLVRYDDIEETNYFQQASELAKLVNQEVLADPLVPPQLTIKDFYMTDSISRASQTMAKCVKAVTEGAQAVEEPSIC

SEQ ID NO: 68 - protein, 364 aa, Homo sapiens
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 69 - protein, 364 aa, Homo sapiens
MPHSYPALSAEQKKELSDIALRIVAPGKGILAADESVGSMAKRLSQIGVENTEENRRLYRQVLFSADDRVKKCIGGVIFFHETLYQKDDNGVPFVRTIQDKGIVVGIKVDKGVVPLAGTDGETTTQGLDGLSERCAQYKKDGADFAKWRCVLKISERTPSALAILENANVLARYASICQQNGIVPIVEPEILPDGDHDLKRCQYVTEKVLAAVYKALSDHHVYLEGTLLKPNMVTPGHACPIKYTPEEIAMATVTALRRTVPPAVPGVTFLSGGQSEEEASFNLNAINRCPLPRPWALTFSYGRALQASALNAWRGQRDNAGAATEEFIKRAEVNGLAAQGKYEGSGEDGGAAAQSLYIANHAY

SEQ ID NO: 70 - protein, 249 aa, Homo sapiens
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 71 - protein, 335 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 72 - protein, 408 aa, Homo sapiens
MSKRDIVLTNVTVVQLLRQPCPVTRAPPPPEPKAEVEPQPQPEPTPVREEIKPPPPPLPPHPATPPPKMVSVARELTVGINGFGRIGRLVLRACMEKGVKVVAVNDPFIDPEYMVYMFKYDSTHGRYKGSVEFRNGQLVVDNHEISVYQCKEPKQIPWRAVGSPYVVESTGVYLSIQAASDHISAGAQRVVISAPSPDAPMFVMGVNENDYNPGSMNIVSNASCTTNCLAPLAKVIHERFGIVEGLMTTVHSYTATQKTVDGPSRKAWRDGRGAHQNIIPASTGAAKAVTKVIPELKGKLTGMAFRVPTPDVSVVDLTCRLAQPAPYSAIKEAVKAAAKGPMAGILAYTEDEVVSTDFLGDTHSSIFDAKAGIALNDNFVKLISWYDNEYGYSHRVVDLLRYMFSRDK

SEQ ID NO: 73 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 74 - protein, 434 aa, Homo sapiens
MSIEKIWAREILDSRGNPTVEVDLYTAKGLFRAAVPSGASTGIYEALELRDGDKQRYLGKGVLKAVDHINSTIAPALISSGLSVVEQEKLDNLMLELDGTENKSKFGANAILGVSLAVCKAGAAERELPLYRHIAQLAGNSDLILPVPAFNVINGGSHAGNKLAMQEFMILPVGAESFRDAMRLGAEVYHTLKGVIKDKYGKDATNVGDEGGFAPNILENSEALELVKEAIDKAGYTEKIVIGMDVAASEFYRDGKYDLDFKSPTDPSRYITGDQLGALYQDFVRDYPVVSIEDPFDQDDWAAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGDEARFAGHNFRNPSVL

SEQ ID NO: 75 - protein, 334 aa, Homo sapiens
MATLKEKLIAPVAEEEATVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 76 - protein, 727 aa, Homo sapiens
MAFQKAVKGTILVGGGALATVLGLSQFAHYRRKQMNLAYVKAADCISEPVNREPPSREAQLLTLQNTSEFDILVIGGGATGSGCALDAVTRGLKTALVERDDFSSGTSSRSTKLIHGGVRYLQKAIMKLDIEQYRMVKEALHERANLLEIAPHLSAPLPIMLPVYKWWQLPYYWVGIKLYDLVAGSNCLKSSYVLSKSRALEHFPMLQKDKLVGAIVYYDGQHNDARMNLAIALTAARYGAATANYMEVVSLLKKTDPQTGKVHVSGARCKDVLTGQEFDVRAKCVINATGPFTDSVRKMDDKDAAAICQPSAGVHIVMPGYYSPESMGLLDPATSDGRVIFFLPWQKMTIAGTTDTPTDVTHHPIPSEEDINFILNEVRNYLSCDVEVRRGDVLAAWSGIRPLVTDPKSADTQSISRNHVVDISESGLITIAGGKWTTYRSMAEDTINAAVKTHNLKAGPSRTVGLFLQGGKDWSPTLYIRLVQDYGLESEVAQHLAATYGDKAFEVAKMASVTGKRWPIVGVRLVSEFPYIEAEVKYGIKEYACTAVDMISRRTRLAFLNVQAAEEALPRIVELMGRELNWDDYKKQEQLETARKFLYYEMGYKSRSEQLTDRSEISLLPSDIDRYKKRFHKFDADQKGFITIVDVQRVLESINVQMDENTLHEILNEVDLNKNGQVELNEFLQLMSAIQKGRVSGSRLAILMKTAEENLDRRVPIPVDRSCGGL

SEQ ID NO: 77 - protein, 727 aa, Homo sapiens
MAFQKAVKGTILVGGGALATVLGLSQFAHYRRKQMNLAYVKAADCISEPVNREPPSREAQLLTLQNTSEFDILVIGGGATGSGCALDAVTRGLKTALVERDDFSSGTSSRSTKLIHGGVRYLQKAIMKLDIEQYRMVKEALHERANLLEIAPHLSAPLPIMLPVYKWWQLPYYWVGIKLYDLVAGSNCLKSSYVLSKSRALEHFPMLQKDKLVGAIVYYDGQHNDARMNLAIALTAARYGAATANYMEVVSLLKKTDPQTGKVRVSGARCKDVLTGQEFDVRAKCVINATGPFTDSVRKMDDKDAAAICQPSAGVHIVMPGYYSPESMGLLDPATSDGRVIFFLPWQKMTIAGTTDTPTDVTHHPIPSEEDINFILNEVRNYLSCDVEVRRGDVLAAWSGIRPLVTDPKSADTQSISRNHVVDISESGLITIAGGKWTTYRSMAEDTINAAVKTHNLKAGPSRTVGLFLQGGKDWSPTLYIRLVQDYGLESEVAQHLAATYGDKAFEVAKMASVTGKRWPIVGVRLVSEFPYIEAEVKYGIKEYACTAVDMISRRTRLAFLNVQAAEEALPRIVELMGRELNWDDYKKQEQLETARKFLYYEMGYKSRSEQLTDRSEISLLPSDIDRYKKRFHKFDADQKGFITIVDVQRVLESINVQMDENTLHEILNEVDLNKNGQVELNEFLQLMSAIQKGRVSGSRLAILMKTAEENLDRRVPIPVDRSCGGL

SEQ ID NO: 78 - protein, 373 aa, Homo sapiens
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 79 - protein, 373 aa, Homo sapiens; identical to SEQ ID NO: 78

SEQ ID NO: 80 - protein, 647 aa, Homo sapiens
MWRVCARRAQNVAPWAGLEARWTALQEVPGTPRVTSRSGPAPARRNSVTTGYGGVRALCGWTPSSGATPRNRLLLQLLGSPGRRYYSLPPHQKVPLPSLSPTMQAGTIARWEKKEGDKINEGDLIAEVETDKATVGFESLEECYMAKILVAEGTRDVPIGAIICITVGKPEDIEAFKNYTLDSSAAPTPQAAPAPTPAATASPPTPSAQAPGSSYPPHMQVLLPALSPTMTMGTVQRWEKKVGEKLSEGDLLAEIETDKATIGFEVQEEGYLAKILVPEGTRDVPLGTPLCIIVEKEADISAFADYRPTEVTDLKPQVPPPTPPPVAAVPPTPQPLAPTPSAPCPATPAGPKGRVFVSPLAKKLAVEKGIDLTQVKGTGPDGRITKKDIDSFVPSKVAPAPAAVVPPTGPGMAPVPTGVFTDIPISNIRRVIAQRLMQSKQTIPHYYLSIDVNMGEVLLVRKELNKILEGRSKISVNDFIIKASALACLKVPEANSSWMDTVIRQNHVVDVSVAVSTPAGLITPIVFNAHIKGVETIANDVVSLATKAREGKLQPHEFQGGTFTISNLGMFGIKNFSAIINPPQACILAIGASEDKLVPADNEKGFDVASMMSVTLSCDHRVVDGAVGAQWLAEFRKYLEKPITMLL

SEQ ID NO: 81 - protein, 366 aa, Homo sapiens
MAGPAWISKVSRLLGAFHNPKQVTRGFTGGVQTVTLIPGDGIGPEISAAVMKIFDAAKAPIQWEERNVTAIQGPGGKWMIPSEAKESMDKNKMGLKGPLKTPIAAGHPSMNLLLRKTFDLYANVRPCVSIEGYKTPYTDVNIVTIRENTEGEYSGIEHVIVDGVVQSIKLITEGASKRIAEFAFEYARNNHRSNVTAVHKANIMRMSDGLFLQKCREVAESCKDIKFNEMYLDTVCLNMVQDPSQFDVLVMPNLYGDILSDLCAGLIGGLGVTPSGNIGANGVAIFESVHGTAPDIAGKDMANPTALLLSAVMMLRHMGLFDHAARIEAACFATIKDGKSLTKDLGGNAKCSDFTEEICRRVKDLD

SEQ ID NO: 82 - protein, 334 aa, Homo sapiens
MSEPIRVLVTGAAGQIAYSLLYSIGNGSVFGKDQPIILVLLDITPMMGVLDGVLMELQDCALPLLKDVIATDKEDVAFKDLDVAILVGSMPRREGMERKDLLKANVKIFKSQGAALDKYAKKSVKVIVVGNPANTNCLTASKSAPSIPKENFSCLTRLDHNRAKAQIALKLGVTANDVKNVIIWGNHSSTQYPDVNHAKVKLQGKEVGVYEALKDDSWLKGEFVTTVQQRGAAVIKARKLSSAMSAAKAICDHVRDIWFGTPEGEFVSMGVISDGNSYGVPDDLLYSFPVVIKNKTWKFVEGLPINDFSREKMDLTAKELTEEKESAFEFLSSA

SEQ ID NO: 83 - protein, 172 aa, Homo sapiens
MPGIVELPTLEELKVDEVKISSAVLKAAAHHYGAQCDKPNKEFMLCRWEEKDPRRCLEEGKLVNKCALDFFRQIKRHCAEPFTEYWTCIDYTGQQLFRHCRKQQAKFDECVLDKLGWVRPDLGELSKVTKVKTDRPLPENPYHSRPRPDPSPEIEGDLQPATHGSRFYFWTK

SEQ ID NO: 84 - protein, 727 aa, Homo sapiens
MLRIPVRKALVGLSKSPKGCVRTTATAASNLIEVFVDGQSVMVEPGTTVLQACEKVGMQIPRFCYHERLSVAGNCRMCLVEIEKAPKVVAACAMPVMKGWNILTNSEKSKKAREGVMEFLLANHPLDCPICDQGGECDLQDQSMMFGNDRSRFLEGKRAVEDKNIGPLVKTIMTRCIQCTRCIRFASEIAGVDDLGTTGRGNDMQVGTYIEKMFMSELSGNIIDICPVGALTSKPYAFTARPWETRKTESIDVMDAVGSNIVVSTRTGEVMRILPRMHEDINEEWISDKTRFAYDGLKRQRLTEPMVRNEKGLLTYTSWEDALSRVAGMLQSFQGKDVAAIAGGLVDAEALVALKDLLNRVDSDTLCTEEVFPTAGAGTDLRSNYLLNTTIAGVEEADVVLLVGTNPRFEAPLFNARIRKSWLHNDLKVALIGSPVDLTYTYDHLGDSPKILQDIASGSHPFSQVLKEAKKPMVVLGSSALQRNDGAAILAAVSSIAQKIRMTSGVTGDWKVMNILHRIASQVAALDLGYKPGVEAIRKNPPKVLFLLGADGGCITRQDLPKDCFIIYQGHHGDVGAPIADVILPGAAYTEKSATYVNTEGRAQQTKVAVTPPGLAREDWKIIRALSEIAGMTLPYDTLDQVRNRLEEVSPNLVRYDDIEGANYFQQANELSKLVNQQLLADPLVPPQLTIKDFYMTDSISRASQTMAKCVKAVTEGAQAVEEPSIC

SEQ ID NO: 85 - protein, 210 aa, Homo sapiens
MRCLTTPMLLRALAQAARAGPPGGRSLHSSAVAATYKYVNMQDPEMDMKSVTDRAARTLLWTELFRGLGMTLSYLFREPATINYPFEKGPLSPRFRGEHALRRYPSGEERCIACKLCEAICPAQAITIEAEPRADGSRRTTRYDIDMTKCIYCGFCQEACPVDAIVEGPNFEFSTETHEELLYNKEKLLNNGDKWEAEIAANIQADYLYR

SEQ ID NO: 86 - protein, 480 aa, Homo sapiens
MAASVVCRAATAGAQVLLRARRSPALLRTPALRSTATFAQALQFVPETQVSLLDNGLRVASEQSSQPTCTVGVWIDVGSRFETEKNNGAGYFLEHLAFKGTKNRPGSALEKEVESMGAHLNAYSTREHTAYYIKALSKDLPKAVELLGDIVQNCSLEDSQIEKERDVILREMQENDASMRDVVFNYLHATAFQGTPLAQAVEGPSENVRKLSRADLTEYLSTHYKAPRMVLAAAGGVEHQQLLDLAQKHLGGIPWTYAEDAVPTLTPCRFTGSEIRHRDDALPFAHVAIAVEGPGWASPDNVALQVANAIIGHYDCTYGGGVHLSSPLASGAVANKLCQSFQTFSICYAETGLLGAHFVCDRMKIDDMMFVLQGQWMRLCTSATESEVARGKNILRNALVSHLDGTTPVCEDIGRSLLTYGRRIPLAEWESRIAEVDASVVREICSKYIYDQCPAVAGYGPIEQLPDYNRIRSGMFWLRF

SEQ ID NO: 87 - protein, 161 aa, Homo sapiens
MAGRKLALKTIDWVAFAEIIPQNQKAIASSLKSWNETLTSRLAALPENPPAIDWAYYKANVAKAGLVDDFEKKFNALKVPVPEDKYTAQVDAEEKEDVKSCAEWVSLSKARIVEYEKEMEKMKNLIPFDQMTIEDLNEAFPETKLDKKKYPYWPHQPIENL

SEQ ID NO: 88 - protein, 137 aa, Homo sapiens
MAGRKLALKTIDWVAFAEIIPQNQKAIASSLKSWNETLTSRLAALPENPPAIDWAYYKANVAKAGLVDDFEKKVKSCAEWVSLSKARIVEYEKEMEKMKNLIPFDQMTIEDLNEAFPETKLDKKKYPYWPHQPIENL

SEQ ID NO: 89 - protein, 381 aa, Homo sapiens
MPFSNSHNALKLRFPAEDEFPDLSAHNNHMAKVLTPELYAELRAKSTPSGFTLDDVIQTGVDNPGHPYIMTVGCVAGDEESYEVFKDLFDPIIEDRHGGYKPSDEHKTDLNPDNLQGGDDLDPNYVLSSRVRTGRSIRGFCLPPHCSRGERRAIEKLAVEALSSLDGDLAGRYYALKSMTEAEQQQLIDDHFLFDKPVSPLLLASGMARDWPDARGIWHNDNKTFLVWVNEEDHLRVISMQKGGNMKEVFTRFCTGLTQIETLFKSKDYEFMWNPHLGYILTCPSNLGTGLRAGVHIKLPNLGKHEKFSEVLKRLRLQKRGTGGVDTAAVGGVFDVSNADRLGFSEVELVQMVVDGVKLLIEMEQRLEQGQAIDDLMPAQK

SEQ ID NO: 90 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 91 - protein, 493 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGMPGGMPGGFPGGGAPPSGGASSGPTIEEVD

SEQ ID NO: 92 - protein, 679 aa, Homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 93 - protein, 505 aa, Homo sapiens
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 94 - protein, 617 aa, Homo sapiens
MDFSKLPKILDEDKESTFGYVHGVSGPVVTACDMAGAAMYELVRVGHSELVGEIIRLEGDMATIQVYEETSGVSVGDPVLRTGKPLSVELGPGIMGAIFDGIQRPLSDISSQTQSIYIPRGVNVSALSRDIKWDFTPCKNLRVGSHITGGDIYGIVSENSLIKHKIMLPPRNRGTVTYIAPPGNYDTSDVVLELEFEGVKEKFTMVQVWPVRQVRPVTEKLPANHPLLTGQRVLDALFPCVQGGTTAIPGAFGCGKTVISQSLSKYSNSDVIIYVGCGERGNEMSEVLRDFPELTMEVDGKVESIMKRTALVANTSNMPVAAREASIYTGITLSEYFRDMGYHVSMMADSTSRWAEALREISGRLAEMPADSGYPAYLGARLASFYERAGRVKCLGNPEREGSVSIVGAVSPPGGDFSDPVTSATLGIVQVFWGLDKKLAQRKHFPSVNWLISYSKYMRALDEYYDKHFTEFVPLRTKAKEILQEEEDLAEIVQLVGKASLAETDKITLEVAKLIKDDFLQQNGYTPYDRFCPFYKTVGMLSNMIAFYDMARRAVETTAQSDNKITWSIIREHMGDILYKLSSMKFKDPLKDGEAKIKSDYAQLLEDMQNAFRSLED

SEQ ID NO: 95 - protein, 418 aa, Homo sapiens
MEEIGILVEKAQDEIPALSVSRPQTGLSFLGPEPEDLEDLYSRYKKLQQELEFLEVQEEYIKDEQKNLKKEFLHAQEEVKRIQSIPLVIGQFLEAVDQNTAIVGSTTGSNYYVRILSTIDRELLKPNASVALHKHSNALVDVLPPEADSSIMMLTSDQKPDVMYADIGGMDIQKQEVREAVELPLTHFELYKQIGIDPPRGVLMYGPPGCGKTMLAKAVAHHTTAAFIRVVGSEFVQKYLGEGPRMVRDVFRLAKENAPAIIFIDEIDAIATKRFDAQTGADREVQRILLELLNQMDGFDQNVNVKVIMATNRADTLDPALLRPGRLDRKIEFPLPDRRQKRLIFSTITSKMNLSEEVDLEDYVARPDKISGADINSICQESGMLAVRENRYIVLAKDFEKAYKTVIKKDEQEHEFYK

SEQ ID NO: 96 - protein, 387 aa, Homo sapiens
MEEIGILVEKAQDEIPALSVSRPQTGLSFLGPEPEDLEDLYSRYKEEVKRIQSIPLVIGQFLEAVDQNTAIVGSTTGSNYYVRILSTIDRELLKPNASVALHKHSNALVDVLPPEADSSIMMLTSDQKPDVMYADIGGMDIQKQEVREAVELPLTHFELYKQIGIDPPRGVLMYGPPGCGKTMLAKAVAHHTTAAFIRVVGSEFVQKYLGEGPRMVRDVFRLAKENAPAIIFIDEIDAIATKRFDAQTGADREVQRILLELLNQMDGFDQNVNVKVIMATNRADTLDPALLRPGRLDRKIEFPLPDRRQKRLIFSTITSKMNLSEEVDLEDYVARPDKISGADINSICQESGMLAVRENRYIVLAKDFEKAYKTVIKKDEQEHEFYK

SEQ ID NO: 97 - protein, 234 aa, Homo sapiens
MAERGYSFSLTTFSPSGKLVQIEYALAAVAGGAPSVGIKAANGVVLATEKKQKSILYDERSVHKVEPITKHIGLVYSGMGPDYRVLVHRARKLAQQYYLVYQEPIPTAQLVQRVASVMQEYTQSGGVRPFGVSLLICGWNEGRPYLFQSDPSGAYFAWKATAMGKNYVNGKTFLEKRYNEDLELEDAIHTAILTLKESFEGQMTEDNIEVGICNEAGFRRLTPTEVKDYLAAIA

SEQ ID NO: 98 - protein, 223 aa, Homo sapiens [sequence omitted]

SEQ ID NO: 99 - protein, 806 aa, Homo sapiens
MASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYG

SEQ ID NO: 100 - protein, 336 aa, Homo sapiens
MAASLRLLGAASGLRYWSRRLRPAAGSFAAVCSRSVASKTPVGFIGLGNMGNPMAKNLMKHGYPLIIYDVFPDACKEFQDAGEQVVSSPADVAEKADRIITMLPTSINAIEAYSGANGILKKVKKGSLLIDSSTIDPAVSKELAKEVEKMGAVFMDAPVSGGVGAARSGNLTFMVGGVEDEFAAAQELLGCMGSNVVYCGAVGTGQAAKICNNMLLAISMIGTAEAMNLGIRLGLDPKLLAKILNMSSGRCWSSDTYNPVPGVMDGVPSANNYQGGFGTTLMAKDLGLAQDSATSTKSPILLGSLAHQIYRMMCAKGYSKKDFSSVFQFLREEETF

SEQ ID NO: 101 - protein, 291 aa, Homo sapiens
MVAVLGGRGVLRLRLLLSALKPGIHVPRAGPAAAFGTSVTSAKVAVNGVQLHYQQTGEGDHAVLLLPGMLGSGETDFGPQLKNLNKKLFTVVAWDPRGYGHSRPPDRDFPADFFERDAKDAVDLMKALKFKKVSLLGWSDGGITALIAAAKYPSYIHKMVIWGANAYVTDEDSMIYEGIRDVSKWSERTRKPLEALYGYDYFARTCEKWVDGIRQFKHLPDGNICRHLLPRVQCPALIVHGEKDPLVPRFHADFIHKHVKGSRLHLMPEGKHNLHLRFADEFNKLAEDFLQ

SEQ ID NO: 102 - protein, 259 aa, Homo sapiens
MAACRALKAVLVDLSGTLHIEDAAVPGAQEALKRLRGASVIIRFVTNTTKESKQDLLERLRKLEFDISEDEIFTSLTAARSLLERKQVRPMLLVDDRALPDFKGIQTSDPNAVVMGLAPEHFHYQILNQAFRLLLDGAPLIAIHKARYYKRKDGLALGPGPFVTALEYATDTKATVVGKPEKTFFLEALRGTGCEPEEAVMIGDDCRDDVGGAQDVGMLGILVKTGKYRASDEEKINPPPYLTCESFPHAVDHILQHLL

SEQ ID NO: 103 - protein, 375 aa, Homo sapiens
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 104 - protein, 375 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 105 - protein, 140 aa, Homo sapiens
MAGWQSYVDNLMCDGCCQEAAIVGYCDAKYVWAATAGGVFQSITPIEIDMIVGKDREGFFTNGLTLGAKKCSVIRDSLYVDGDCTMDIRTKSQGGEPTYNVAVGRAGRVLVFVMGKEGVHGGGLNKKAYSMAKYLRDSGF

SEQ ID NO: 106 - protein, 140 aa, Homo sapiens
MAGWQSYVDNLMCDGCCQEAAIVGYCDAKYVWAATAGGVFQSITPIEIDMIVGKDREGFFTNGLTLGAKKCSVIRDSLYVDGDCTMDIRTKSQGGEPTYNVAVGRAGRALVIVMGKEGVHGGTLNKKAYELALYLRRSDV

SEQ ID NO: 107 - protein, 199 aa, Homo sapiens
MANRGPSYGLSREVQEKIEQKYDADLENKLVDWIILQCAEDIEHPPPGRAHFQKWLMDGTVLCKLINSLYPPGQEPIPKISESKMAFKQMEQISQFLKAAETYGVRTTDIFQTVDLWEGKDMAAVQRTLMALGSVAVTKDDGCYRGEPSWFHRKAQQNRRGFSEEQLRQGQNVIGLQMGSNKGASQAGMTGYGMPRQIM

SEQ ID NO: 108 - protein, 673 aa, Homo sapiens
MAKPAQGAKYRGSIHDFPGFDPNQDAEALYTAMKGFGSDKEAILDIITSRSNRQRQEVCQSYKSLYGKDLIADLKYELTGKFERLIVGLMRPPAYCDAKEIKDAISGIGTDEKCLIEILASRTNEQMHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTPEYFAERLFKAMKGLGTRDNTLIRIMVSRSELDMLDIREIFRTKYEKSLYSMIKNDTSGEYKKTLLKLSGGDDDAAGQFFPEAAQVAYQMWELSAVARVELKGTVRPANDFNPDADAKALRKAMKGLGTDEDTIIDIITHRSNVQRQQIRQTFKSHFGRDLMTDLKSEISGDLARLILGLMMPPAHYDAKQLKKAMEGAGTDEKALIEILATRTNAEIRAINEAYKEDYHKSLEDALSSDTSGHFRRILISLATGHREEGGENLDQAREDAQVAAEILEIADTPSGDKTSLETRFMTILCTRSYPHLRRVFQEFIKMTNYDVEHTIKKEMSGDVRDAFVAIVQSVKNKPLFFADKLYKSMKGAGTDEKTLTRIMVSRSEIDLLNIRREFIEKYDKSLHQAIEGDTSGDFLKALLALCGGED

SEQ ID NO: 109 - protein, 667 aa, Homo sapiens
MAKPAQGAKYRGSIHDFPGFDPNQDAEALYTAMKGFGSDKEAILDIITSRSNRQRQEVCQSYKSLYGKDLIADLKYELTGKFERLIVGLMRPPAYCDAKEIKDAISGIGTDEKCLIEILASRTNEQMHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTPEYFAERLFKAMKGLGTRDNTLIRIMVSRSELDMLDIREIFRTKYEKSLYSMIKNDTSGEYKKTLLKLSGGDDDAAGQFFPEAAQVAYQMWELSAVARVELKGTVRPANDFNPDADAKALRKAMKGLGTDEDTIIDIITHRSNVQRQQIRQTFKSHFGRDLMTDLKSEISGDLARLILGLMMPPAHYDAKQLKKAMEGAGTDEKALIEILATRTNAEIRAINEAYKEDYHKSLEDALSSDTSGHFRRILISLATGHREEGGENLDQAREDAQEIADTPSGDKTSLETRFMTILCTRSYPHLRRVFQEFIKMTNYDVEHTIKKEMSGDVRDAFVAIVQSVKNKPLFFADKLYKSMKGAGTDEKTLTRIMVSRSEIDLLNIRREFIEKYDKSLHQAIEGDTSGDFLKALLALCGGED

SEQ ID NO: 110 - protein, 499 aa, Homo sapiens
MSFGSEHYLCSSSSYRKVFGDGSRLSARLSGAGGAGGFRSQSLSRSNVASSAACSSASSLGLGLAYRRPPASDGLDLSQAAARTNEYKIIRTNEKEQLQGLNDRFAVFIEKVHQLETQNRALEAELAALRQRHAEPSRVGELFQRELRDLRAQLEEASSARSQALLERDGLAEEVQRLRARCEEESRGREGAERALKAQQRDVDGATLARLDLEKKVESLLDELAFVRQVHDEEVAELLATLQASSQAAAEVDVTVAKPDLTSALREIRAQYESLAAKNLQSAEEWYKSKFANLNEQAARSTEAIRASREEIHEYRRQLQARTIEIEGLRGANESLERQILELEERHSAEVAGYQDSIGQLENDLRNTKSEMARHLREYQDLLNVKMALDIEIAAYRKLLEGEETRFSTSGLSISGLNPLPNPSYLLPPRILSATTSKVSSTGLSLKKEEEEEEASKVASKKTSQIGESFEEILEETVISTKKTEKSNIEETTISSQKI

SEQ ID NO: 111 - protein, 543 aa, Homo sapiens
MSSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD

SEQ ID NO: 112 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 113 - protein, 431 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRGGKSTKDGENHKVTRYLKSLTIRVIPIQAHQIVNGTPPARG

SEQ ID NO: 114 - protein, 451 aa, Homo sapiens [sequence omitted]

SEQ ID NO: 115 - protein, 445 aa, Homo sapiens [sequence omitted]

SEQ ID NO: 116 - protein, 450 aa, Homo sapiens
MREIVHIQAGQCGNQIGAKFWEVISDEHGIDPSGNYVGDSDLQLERISVYYNEASSHKYVPRAILVDLEPGTMDSVRSGAFGHLFRPDNFIFGQSGAGNNWAKGHYTEGAELVDSVLDVVRKECENCDCLQGFQLTHSLGGGTGSGMGTLLISKVREEYPDRIMNTFSVVPSPKVSDTVVEPYNATLSIHQLVENTDETYCIDNEALYDICFRTLKLATPTYGDLNHLVSATMSGVTTSLRFPGQLNADLRKLAVNMVPFPRLHFFMPGFAPLTARGSQQYRALTVPELTQQMFDAKNMMAACDPRHGRYLTVATVFRGRMSMKEVDEQMLAIQSKNSSYFVEWIPNNVKVAVCDIPPRGLKMSSTFIGNSTAIQELFKRISEQFTAMFRRKAFLHWYTGEGMDEMEFTEAESNMNDLVSEYQQYQDATAEEEGEMYEDDEEESEAQGPK

SEQ ID NO: 117 - protein, 572 aa, Homo sapiens
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRIAVGSDADLVIWDPDSVKTISAKTHNSSLEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 118 - protein, 572 aa, Homo sapiens
MSFQGKKSIPRITSDRLLIRGGRIVNDDQSFYADVHVEDGLIKQIGENLIVPGGIKTIDAHGLMVLPGGVDVHTRLQMPVLGMTPADDFCQGTKAALAGGTTMILDHVFPDTGVSLLAAYEQWRERADSAACCDYSLHVDITRWHESIKEELEALVKEKGVNSFLVFMAYKDRCQCSDSQMYEIFSIIRDLGALAQVHAENGDIVEEEQKRLLELGITGPEGHVLSHPEEVEAEAVYRAVTIAKQANCPLYVTKVMSKGAADAIAQAKRRGVVVFGEPITASLGTDGSHYWSKNWAKAAAFVTSPPVNPDPTTADHLTCLLSSGDLQVTGSAHCTFTTAQKAVGKDNFALIPEGTNGIEERMSMVWEKCVASGKMDENEFVAVTSTNAAKIFNFYPRKGRVAVGSDADLVIWNPKATKIISAKTHNLNVEYNIFEGVECRGAPAVVISQGRVALEDGKMFVTPGAGRFVPRKTFPDFVYKRIKARNRLAEIHGVPRGLYDGPVHEVMVPAKPGSGAPARASCPGKISVPPVRNLHQSGFSLSGSQADDHIARRTAQKIMAPPGGRSNITSLS

SEQ ID NO: 119 - protein, 227 aa, Homo sapiens
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 120 - protein, 201 aa, Homo sapiens
MNPEYDYLFKLLLIGDSGVGKSCLLLRFADDTYTESYISTIGVDFKIRTIELDGKTIKLQIWDTAGQERFRTITSSYYRGAHGIIVVYDVTDQESYANVKQWLQEIDRYASENVNKLLVGNKSDLTTKKVVDNTTAKEFADSLGIPFLETSAKNATNVEQAFMTMAAEIKKRMGPGAASGGERPNLKIDSTPVKPAGGGCC

SEQ ID NO: 121 - protein, 220 aa, Homo sapiens
MASATDSRYGQKESSDQNFDYMFKILIIGNSSVGKTSFLFRYADDSFTPAFVSTVGIDFKVKTIYRNDKRIKLQIWDTAGQERYRTITTAYYRGAMGFILMYDITNEESFNAVQDWSTQIKTYSWDNAQVLLVGNKCDMEDERVVSSERGRQLADHLGFEFFEASAKDNINVKQTFERLVDVICEKMSESLDTADPAVTGAKQGPQLSDQQVPPHQDCAC

SEQ ID NO: 122 - protein, 208 aa, Homo sapiens
MSTGGDFGNPLRKFKLVFLGEQSVGKTSLITRFMYDSFDNTYQATIGIDFLSKTMYLEDRTIRLQLWDTAGQERFRSLIPSYIRDSAAAVVVYDITNVNSFQQTTKWIDDVRTERGSDVIIMLVGNKTDLADKRQVSIEEGERKAKELNVMFIETSAKAGYNVKQLFRRVAAALPGMESTQDRSREDMIDIKLEKPQEQPVSEGGCSC

SEQ ID NO: 123 - protein, 447 aa, Homo sapiens [sequence omitted]

SEQ ID NO: 124 - protein, 298 aa, Homo sapiens; identical to SEQ ID NO: 50

SEQ ID NO: 125 - protein, 505 aa, Homo sapiens; identical to SEQ ID NO: 93

SEQ ID NO: 126 - protein, 170 aa, Mus musculus
MGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDV

SEQ ID NO: 127 - protein, 170 aa, Homo sapiens; identical to SEQ ID NO: 126

SEQ ID NO: 128 - protein, 261 aa, Homo sapiens
MAESHLQSSLITASQFFEIWLHFDADGSGYLEGKELQNLIQELQQARKKAGLELSPEMKTFVDQYGQRDDGKIGIVELAHVLPTEENFLLLFRCQQLKSCEEFMKTWRKYDTDHSGFIETEELKNFLKDLLEKANKTVDDTKLAEYTDLMLKLFDSNNDGKLELTEMARLLPVQENFLLKFQGIKMCGKEFNKAFELYDQDGNGYIDENELDALLKDLCEKNKQDLDINNITTYKKNIMALSDGGKLYRTDLALILCAGDN

SEQ ID NO: 129 - protein, 271 aa, Homo sapiens
MAGPQQQPPYLHLAELTASQFLEIWKHFDADGNGYIEGKELENFFQELEKARKGSGMMSKSDNFGEKMKEFMQKYDKNSDGKIEMAELAQILPTEENFLLCFRQHVGSSAEFMEAWRKYDTDRSGYIEANELKGFLSDLLKKANRPYDEPKLQEYTQTILRMFDLNGDGKLGLSEMSRLLPVQENFLLKFQGMKLTSEEFNAIFTFYDKDRSGYIDEHELDALLKDLYEKNKKEMNIQQLTNYRKSVMSLAEAGKLYRKDLEIVLCSEPPM

SEQ ID NO: 130 - protein, 191 aa, Homo sapiens; identical to SEQ ID NO: 55

SEQ ID NO: 131 - protein, 253 aa, Homo sapiens
MALSMPLNGLKEEDKEPLIELFVKAGSDGESIGNCPFSQRLFMILWLKGVVFSVTTVDLKRKPADLQNLAPGTHPPFITFNSEVKTDVNKIEEFLEEVLCPPKYLKLSPKHPESNTAGMDIFAKFSAYIKNSRPEANEALERGLLKTLQKLDEYLNSPLPDEIDENSMEDIKFSTRKFLDGNEMTLADCNLLPKLHIVKVVAKKYRNFDIPKEMTGIWRYLTNAYSRDEFTNTCPSDKEVEIAYSDVAKRLTK

SEQ ID NO: 132 - protein, 187 aa, Homo sapiens
MPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGK

SEQ ID NO: 133 - protein, 245 aa, Homo sapiens
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 134 - protein, 255 aa, Homo sapiens
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 135 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 136 - protein, 256 aa, Homo sapiens
MAAAVGRLLRASVARHVSAIPWGISATAALRPAACGRTSLTNLLCSGSSQAKLFSTSSSCHAPAVTQHAPYFKGTAVVNGEFKDLSLDDFKGKYLVLFFYPLDFTFVCPTEIVAFSDKANEFHDVNCEVVAVSVDSHFSHLAWINTPRKNGGLGHMNIALLSDLTKQISRDYGVLLEGSGLALRGLFIIDPNGVIKHLSVNDLPVGRSVEETLRLVKAFQYVETHGEVCPANWTPDSPTIKPSPAASKEYFQKVNQ

SEQ ID NO: 137 - protein, 312 aa, Homo sapiens
MEEECRVLSIQSHVIRGYVGNRAATFPLQVLGFEIDAVNSVQFSNHTGYAHWKGQVLNSDELQELYEGLRLNNMNKYDYVLTGYTRDKSFLAMVVDIVQELKQQNPRLVYVCDPVLGDKWDGEGSMYVPEDLLPVYKEKVVPLADIITPNQFEAELLSGRKIHSQEEALRVMDMLHSMGPDTVVITSSDLPSPQGSNYLIVLGSQRRRNPAGSVVMERIRMDIRKVDAVFVGTGDLFAAMLLAWTHKHPNNLKVACEKTVSTLHHVLQRTIQCAKAQAGEGVRPSPMQLELRMVQSKRDIEDPEIVVQATVL

SEQ ID NO: 138 - protein, 354 aa, Homo sapiens
MGCTLSAEERAALERSKAIEKNLKEDGISAAKDVKLLLLGAGESGKSTIVKQMKIIHEDGFSGEDVKQYKPVVYSNTIQSLAAIVRAMDTLGIEYGDKERKADAKMVCDVVSRMEDTEPFSAELLSAMMRLWGDSGIQECFNRSREYQLNDSAKYYLDSLDRIGAADYQPTEQDILRTRVKTTGIVETHFTFKNLHFRLFDVGGQRSERKKWIHCFEDVTAIIFCVALSGYDQVLHEDETTNRMHESLMLFDSICNNKFFIDTSIILFLNKKDLFGEKIKKSPLTICFPEYTGPNTYEDAAAYIQAQFESKNRSPNKEIYCHMTCATDTNNIQVVFDAVTDIIIANNLRGCGLY

SEQ ID NO: 139 - protein, 354 aa, Homo sapiens
MGCTLSAEERAALERSKAIEKNLKEDGISAAKDVKLLLLGAGESGKSTIVKQMKIIHEDGFSGEDVKQYKPVVYSNTIQSLAAIVRAMDTLGIEYGDKERKADAKMVCDVVSRMEDTEPFSAELLSAMMRLWGDSGIQECFNRSREYQLNDSAKYYLDSLDRIGAADYQPTEQDILRTRVKTTGIVETHFTFKNLHFRLFDVGGQRSERKKWIHCFEDVTAIIFCVALSGYDQVLHEDETTNRMHESLKLFDSICNNKWFTDTSIILFLNKKDIFEEKIKKSPLTICFPEYTGPSAFTEAVAYIQAQYESKNKSAHKEIYTHVTCATDTNNIQFVFDAVTDVIIAKNLRGCGLY

SEQ ID NO: 140 - protein, 28 aa, homo sapiens
CPNEFTGDRCQNYVMASFYKHLGIEFME

SEQ ID NO: 141 - protein, 20 aa, homo sapiens
CPNEFTGDRCQNYVMASFYK

SEQ ID NO: 142 - protein, 30 aa, homo sapiens
CPNEFTGDRCQNYVMASFYSTSTPFLSLPE

SEQ ID NO: 143 - protein, 20 aa, homo sapiens
CPNEFTGDRCQNYVMASFYS

SEQ ID NO: 144 - protein, 23 aa, homo sapiens
CQPGFTGARCTENVPMKVQNQEK

SEQ ID NO: 145 - protein, 23 aa, homo sapiens
CQPGFTGARCTENVPMKVQNQES

SEQ ID NO: 146 - protein, 31 aa, homo sapiens
CQPGFTGARCTENVPMKVQNQEKHLGIEFIE

SEQ ID NO: 147 - protein, 63 aa, homo sapiens
SHLVKCAEKEKTFCVNGGECFMVKDLSNPSRYLCKCPNEFTGDRCQNYVMASFYKHLGIEFME

SEQ ID NO: 148 - protein, 55 aa, homo sapiens
SHLVKCAEKEKTFCVNGGECFMVKDLSNPSRYLCKCPNEFTGDRCQNYVMASFYK

SEQ ID NO: 149 - protein, 65 aa, homo sapiens
SHLVKCAEKEKTFCVNGGECFMVKDLSNPSRYLCKCPNEFTGDRCQNYVMASFYSTSTPFLSLPE

SEQ ID NO: 150 - protein, 55 aa, homo sapiens
SHLVKCAEKEKTFCVNGGECFMVKDLSNPSRYLCKCPNEFTGDRCQNYVMASFYS

SEQ ID NO: 151 - protein, 58 aa, homo sapiens
SHLVKCAEKEKTFCVNGGECFMVKDLSNPSRYLCKCQPGFTGARCTENVPMKVQNQEK

SEQ ID NO: 152 - protein, 58 aa, homo sapiens
SHLVKCAEKEKTFCVNGGECFMVKDLSNPSRYLCKCQPGFTGARCTENVPMKVQNQES

SEQ ID NO: 153 - protein, 66 aa, homo sapiens
SHLVKCAEKEKTFCVNGGECFMVKDLSNPSRYLCKCQPGFTGARCTENVPMKVQNQEKHLGIEFIE

SEQ ID NO: 154 - protein, 52 aa, homo sapiens
MSERKEGRGKGKGKKKERGSGKKPESAAGSQSPALPPRLKEMKSQESAAGSK

SEQ ID NO: 155 - protein, 52 aa, homo sapiens
MSERKEGRGKGKGKKKERGSGKKPESAAGSQSPALPPQLKEMKSQESAAGSK

SEQ ID NO: 156 - protein, 91 aa, homo sapiens
PQLKEMKSQESAAGSKLVLRCETSSEYSLRFKWFKNGNELNRKNKPQNIKIQKKPGKSELRINKASLADSGEYMCKVISKLGNDSASANIT

SEQ ID NO: 157 - protein, 126 aa, homo sapiens
SERKEGRGKGKGKKKERGSGKKPESAAGSQSPALPPQLKEMKSQESAAGSKLVLRCETSSEYSLRFKWFKNGNELNRKNKPQNIKIQKKPGKSELRINKASLADSGEYMCKVISKLGNDSASANIT

SEQ ID NO: 158 - protein, 63 aa, homo sapiens
VISKLGNDSASANITIVESNEIITGMPASTEGAYVSSESPIRISVSTEGANTSSSTSTSTTGT

SEQ ID NO: 159 - protein, 1342 aa, homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 160 - protein, 1308 aa, homo sapiens
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRSEIGHSPPPAYTPMSGNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV